SEQUENCE LISTING (54 sequences)

SEQ ID NO: 1 - DNA, 57 bp, Artificial
  Oligonucleotide primer
catgccatgactcgcggcccagccggccatggccgaggtgcagctggtggagtctgg

SEQ ID NO: 2 - DNA, 57 bp, Artificial
  Oligonucleotide primer
catgccatgactcgcggcccagccggccatggccgatgtgcagctggtggagtctgg

SEQ ID NO: 3 - DNA, 57 bp, Artificial
  Oligonucleotide primer
catgccatgactcgcggcccagccggccatggccgcggtgcagctggtggagtctgg

SEQ ID NO: 4 - DNA, 57 bp, Artificial
  Oligonucleotide primer
catgccatgactcgcggcccagccggccatggccgccgtgcagctggtggattctgg

SEQ ID NO: 5 - DNA, 57 bp, Artificial
  Oligonucleotide primer
catgccatgactcgcggcccagccggccatggcccaggtgcagctggtggagtctgg

SEQ ID NO: 6 - DNA, 57 bp, Artificial
  Oligonucleotide primer
catgccatgactcgcggcccagccggccatggcccaggtacagctggtggagtctgg

SEQ ID NO: 7 - DNA, 57 bp, Artificial
  Oligonucleotide primer
catgccatgactcgcggcccagccggccatggcccaggtaaagctggaggagtctgg

SEQ ID NO: 8 - protein, 4 aa, Unknown
  Nanobody sequence
GLEW

SEQ ID NO: 9 - protein, 4 aa, Unknown
  Nanobody sequence
KERE

SEQ ID NO: 10 - protein, 4 aa, Unknown
  Nanobody sequence
KQRE

SEQ ID NO: 11 - protein, 4 aa, Unknown
  Nanobody sequence
KLEW

SEQ ID NO: 12 - protein, 5 aa, Unknown
  Nanobody sequence
KEREL

SEQ ID NO: 13 - protein, 5 aa, Unknown
  Nanobody sequence
KEREF

SEQ ID NO: 14 - protein, 5 aa, Unknown
  Nanobody sequence
KQREL

SEQ ID NO: 15 - protein, 5 aa, Unknown
  Nanobody sequence
KQREF

SEQ ID NO: 16 - protein, 5 aa, Unknown
  Nanobody sequence
KEREG

SEQ ID NO: 17 - protein, 4 aa, Unknown
  Nanobody sequence
TERE

SEQ ID NO: 18 - protein, 5 aa, Unknown
  Nanobody sequence
TEREL

SEQ ID NO: 19 - protein, 4 aa, Unknown
  Nanobody sequence
KECE

SEQ ID NO: 20 - protein, 5 aa, Unknown
  Nanobody sequence
KECEL

SEQ ID NO: 21 - protein, 5 aa, Unknown
  Nanobody sequence
KECER

SEQ ID NO: 22 - protein, 4 aa, Unknown
  Nanobody sequence
RERE

SEQ ID NO: 23 - protein, 5 aa, Unknown
  Nanobody sequence
REREG

SEQ ID NO: 24 - protein, 4 aa, Unknown
  Nanobody sequence
QERE

SEQ ID NO: 25 - protein, 5 aa, Unknown
  Nanobody sequence
QEREG

SEQ ID NO: 26 - protein, 4 aa, Unknown
  Nanobody sequence
KGRE

SEQ ID NO: 27 - protein, 5 aa, Unknown
  Nanobody sequence
KGREG

SEQ ID NO: 28 - protein, 4 aa, Unknown
  Nanobody sequence
KDRE

SEQ ID NO: 29 - protein, 5 aa, Unknown
  Nanobody sequence
KDREV

SEQ ID NO: 30 - protein, 5 aa, Unknown
  Nanobody sequence
DECKL

SEQ ID NO: 31 - protein, 5 aa, Unknown
  Nanobody sequence
NVCEL

SEQ ID NO: 32 - protein, 4 aa, Unknown
  Nanobody sequence
GVEW

SEQ ID NO: 33 - protein, 4 aa, Unknown
  Nanobody sequence
EPEW

SEQ ID NO: 34 - protein, 4 aa, Unknown
  Nanobody sequence
GLER

SEQ ID NO: 35 - protein, 4 aa, Unknown
  Nanobody sequence
DQEW

SEQ ID NO: 36 - protein, 4 aa, Unknown
  Nanobody sequence
DLEW

SEQ ID NO: 37 - protein, 4 aa, Unknown
  Nanobody sequence
GIEW

SEQ ID NO: 38 - protein, 4 aa, Unknown
  Nanobody sequence
ELEW

SEQ ID NO: 39 - protein, 4 aa, Unknown
  Nanobody sequence
GPEW

SEQ ID NO: 40 - protein, 4 aa, Unknown
  Nanobody sequence
EWLP

SEQ ID NO: 41 - protein, 4 aa, Unknown
  Nanobody sequence
GPER

SEQ ID NO: 42 - protein, 4 aa, Unknown
  Nanobody sequence
GLER

SEQ ID NO: 43 - protein, 26 aa, Unknown
  Nanobody sequence
QVQLQESGGGXVQAGGSLRLSCAASG

SEQ ID NO: 44 - protein, 14 aa, Unknown
  Nanobody sequence
WXRQAPGKXXEXVA

SEQ ID NO: 45 - protein, 32 aa, Unknown
  Nanobody sequence
RFTISRDNAKNTVYLQMNSLXXEDTAVYYCAA

SEQ ID NO: 46 - protein, 11 aa, Unknown
  Nanobody sequence
XXQGTXVTVSS

SEQ ID NO: 47 - protein, 15 aa, Artificial
  Linker sequence
GGGGSGGGGSGGGGS

SEQ ID NO: 48 - protein, 15 aa, Artificial
  Linker sequence
GGGSSGGGSSGGGSS

SEQ ID NO: 49 - protein, 4 aa, Artificial
  Poly-arginine sequence
RRRR

SEQ ID NO: 50 - protein, 5 aa, Artificial
  Poly-arginine sequence
RRRRR

SEQ ID NO: 51 - DNA, 50 bp, Artificial
  Oligonucleotide primer
tgactcgcggcccagccggccatggccgaggtgcagctggtggagtctgg

SEQ ID NO: 52 - DNA, 23 bp, Artificial
  Oligonucleotide primer
ggtacgtgctgttgaactgttcc

SEQ ID NO: 53 - DNA, 50 bp, Artificial; identical to SEQ ID NO: 51
  Oligonucleotide primer

SEQ ID NO: 54 - DNA, 18 bp, Artificial
  Oligonucleotide primer
tgaggagacggtgacctg

(19) United States
(12) Patent Application Publication
(10) Pub. No.: US 2006/0211088 A1
(43) Pub. Date: Sep. 21, 2006

(54) Method for generating variable domain sequences of heavy chain antibodies

(75) Inventors: Guy Hermans, Zwijnaarde (BE); Johannes Joseph Wilhelmus De Haard, Oudelande (NL)

Correspondence Address: WOLF GREENFIELD & SACKS, PC, FEDERAL RESERVE PLAZA, 600 ATLANTIC AVENUE, BOSTON, MA, 02210-2206, US

(73) Assignee: Ablynx N.V., Ghent-Zwijnaarde (BE)

(21) Appl. No.: 11/375,679

(22) Filed: Mar. 13, 2006

Related U.S. Application Data
(63) Continuation-in-part of application No. 11/343,972, filed on Jan. 31, 2006.
Application No. 11/343,972 is a continuation of application No. PCT/EP05/11819, filed on Nov. 4, 2005.
(60) Provisional application No. 60/648,922, filed on Jan. 31, 2005.
(60) Provisional application No. 60/663,622, filed on Mar. 18, 2005.

(51) Int. Cl. C07H 21/04; C12P 21/06; C07K 16/18; C12N 5/06

(52) U.S. Cl. 435/69.1; 435/320.1; 435/326; 530/387.1; 536/23.53

(57) ABSTRACT

The present invention relates to a method for generating or cloning a nucleic acid or nucleotide sequence that encodes a heavy chain antibody or an antigen-binding fragment thereof, wherein said heavy chain antibody or antigen-binding fragment is directed against a specific antigen, said method comprising the steps of providing a sample or population of cells from a Camelid immunized with said antigen, isolating from said sample or population said at least one cell that expresses or is capable of expressing a heavy chain antibody directed against said antigen, and obtaining from said at least one cell a nucleic acid or nucleotide sequence that encodes a heavy chain antibody directed against antigen or that encodes an antigen-binding fragment thereof directed against said antigen.

### RELATED APPLICATIONS

This application is a continuation-in-part of U.S. patent application Ser. No. 11/343,972, filed Jan. 31, 2006, which is a continuation of PCT/EP2005/0011819, filed Nov. 4, 2005, which claims the benefit under 35 U.S.C. § 119(e) of U.S. provisional application 60/648,922, filed Jan. 31, 2005, and U.S. provisional application 60/663,622, filed Mar. 18, 2005, the entire disclosures of which are incorporated herein by reference.

The present invention relates to a method for identifying, selecting, generating, cloning and/or producing immunoglobulin sequences.

In particular, the present invention relates to a method for identifying, selecting, generating and/or cloning immunoglobulin sequences (as defined below), wherein said immunoglobulin sequences are heavy chain antibodies (as defined below) or antigen-binding fragments thereof.

More in particular, the present invention relates to a method for identifying, selecting, generating and/or cloning variable domain sequences of heavy chain antibodies.

According to one preferred, but non-limiting aspect, the present invention relates to a method for identifying, selecting, generating and/or cloning (collectively below also “obtaining”) nucleic acids and/or nucleotide sequences that code for heavy chain antibodies or antigen-binding fragments thereof, and in particular for variable domains of heavy chain antibodies, wherein said heavy chain antibodies or antigen-binding fragments thereof are directed against (as defined below) a specific antigen.

The invention also relates to the nucleic acids/nucleotide sequences obtained by the methods of the invention; to genetic constructs comprising or containing the same; to host cells containing and/or expressing the same; and to uses of said nucleic acids/nucleotide sequences, of said genetic constructs and/or of said host cells, for example to produce and/or to express said variable domain sequences.

The invention also relates to the variable domain sequences that are encoded by the nucleic acids/nucleotide sequences of the invention and/or that can be obtained by the expression of said nucleic acids/nucleotide sequences; to uses of said variable domain sequences; and to products or compositions that contain said variable domain sequences.

The invention further relates to proteins and polypeptides that contain or comprise one or more of said variable domain sequences, to uses of such proteins or polypeptides, to products or compositions containing said proteins or polypeptides, and to nucleotide sequences, nucleic acids and/or genetic constructs that encode such proteins or polypeptides.

As also further described below, the variable domain sequences that are obtained using the methods and constructs of the invention can be used as Nanobodies™ [Note: Nanobody™, Nanobodies™ and Nanoclone™ are subject to trademark protection or applications therefor by Ablynx N V.]. In addition, as also described below, the variable domain sequences that are obtained using the methods and constructs of the invention, the amino acid sequences thereof, and/or the nucleic acids and/or nucleotide sequences encoding the same, can be used as a starting point for developing, designing and/or preparing Nanobodies™, e.g. by using one of the various methods outlined in the discussion of the general background art below.

Thus, in a further aspect, the invention also relates to such Nanobodies™; to nucleic acids, nucleotide sequences and/or genetic constructs encoding the same; to uses of such Nanobodies; and to products or compositions that contain such Nanobodies.

The invention further relates to proteins and polypeptides that contain or comprise one or more of said Nanobodies, to uses of such proteins or polypeptides, to products or compositions containing said proteins or polypeptides, and to nucleotide sequences, nucleic acids and/or genetic constructs that encode such proteins or polypeptides. Again, said proteins or polypeptides are preferably in a multivalent or multispecific format.

The invention also relates to methods for preparing the proteins and polypeptides referred to above, and/or to host cells that can express or produce said proteins or polypeptides.

Further aspects, embodiments, uses, applications and advantages of the invention will become clear from the further description hereinbelow.

Discussion of the General Background Art on Heavy Chain Antibodies V_{HH} Domains and Nanobodies.

For a general description of heavy chain antibodies and the variable domains thereof, reference is inter alia made to the following references, which are mentioned as general background art: WO 94/04678 (=EP 656 946), WO 96/34103 (=EP 0 822 985) and WO 97/49805, all by the Vrije Universiteit Brussel; WO 97/49805 by Vlaams Interuniversitair Instituut voor Biotechnologie; WO 94/25591 (=EP 0 698 097) and WO 00/43507 by Unilever N.V.; WO 01/90190 by the National Research Council of Canada; WO 03/025020 (=EP 1 433 793) by the Institute of Antibodies; WO 04/062551, WO 04/041863, WO 04/041865, WO 04/041862 and WO 04/041867 by Ablynx et al.; the non-prepublished International application PCT/BE2004/000159 by Ablynx et al. (filed on Nov. 5, 2004 and entitled “Polypeptides for use in the diagnosis, prophylaxis and treatment of diseases and disorders associated with EGFR and/or CEA, such as cancer”); Hamers-Casterman et al., Nature, Vol. 363, p. 446 (1993); Riechmann and Muyldermans, Journal of Immunological Methods, 231 (1999), p. 25-38; Vu et al., Molecular Immunology, Vol. 34, No. 16-17, p. 1121-1131 (1997); Nguyen et al., EMBO J., Vol. 19, No.5, 921-930 (2000); Arbabi Ghahroudi et al., FEBS Letters 414 (1997) 521-526; van der Linden et al., J. Immunological Methods, 240 (2000), 185-195; Muyldermans, Reviews in Molecular Biotechnology 74 (2001), 277-302; Nguyen et al., Advances in Immunology; Vol. 79 (2001), 261; as well as some of the further references mentioned hereinbelow.

Also, hereinbelow, and in accordance with the terminology used in these references, the variable domains present in naturally occurring heavy chain antibodies will also be referred to as “V_{HH} domains”, in order to distinguish them from the heavy chain variable domains that are present in conventional 4-chain antibodies (which will be referred to hereinbelow as “V_{H} domains”) and from the light chain variable domains that are present in conventional 4-chain antibodies (which will be referred to hereinbelow as “V_{L} domains”). As will become clear from the further discussion below, V_{HH} domains have a number of unique structural characteristics and functional properties, which make isolated V_{HH} domains (as well as Nanobodies, which share said structural characteristics and functional properties with the naturally occurring V_{HH} domains) highly advantageous for use as functional antigen-binding domains or proteins (i.e. compared to isolated naturally occurring V_{H} domains or V_{L} domains, which by themselves are not suitable as antigen-binding units, for the reasons discussed hereinbelow and in the references cited above).

As mentioned in the general background art cited above, heavy chain antibodies are unique in that they do not contain the light chains that are present in naturally occurring conventional 4-chain antibodies (that natively contain both heavy chains and light chains). Nevertheless, because of the way heavy chain antibodies have evolved in nature, they are still capable of binding to an antigen with high affinity and with high specificity (i.e. comparable to the affinity and specificity of conventional 4-chain antibodies).

In other words, heavy chain antibodies do not require the presence of light chains to bind with high affinity and with high specificity to a relevant antigen. This unique feature distinguishes heavy chain antibodies from conventional 4-chain antibodies, which require the interaction between the antigen on the one hand, and both the V_{H} and V_{L} domain on the other hand, to be functional as an antigen-binding unit. [Note: because heavy chain antibodies do not contain light chains, they have also been referred to in the art as “single chain antibodies” (see for example WO 02/085945; and not to be confused with so-called “single chain Fv's” or “scFv's”, which are synthetic polypeptides comprising a V_{H} domain covalently linked to a V_{L} domain) and as “immunoglobulins devoid of light chains” (see for example EP 0 656 946 and some of the further general background art mentioned above), which terms for the purposes of the present description should be considered equivalent to the term “heavy chain antibody”, which will be used herein.]

The above also means that isolated V_{HH} domains—which have been “designed” by nature to functionally bind to an antigen without the presence of, and without any interaction with, a light chain variable domain—can be used as such as a single, relatively small, functional antigen-binding structural unit, domain or protein. This also distinguishes the V_{HH} domains from the V_{H} and V_{L} domains of conventional 4-chain antibodies, which by themselves are generally not suited as antigen-binding proteins or domains, but need to be combined in some form or another to provide a functional antigen-binding unit, as in for example conventional antibody fragments or in scFv's (which consist of a V_{H} domain covalently linked to a V_{L} domain).

Because of these unique properties, the use of V_{HH} domains and Nanobodies as antigen-binding proteins or antigen-binding domains (i.e. as part of a larger protein or polypeptide) offers significant advantages over the use of conventional V_{H} and V_{L} domains, scFv's or conventional antibody fragments (such as Fab- or F(ab)₂-fragments):
only a single domain is required to bind an antigen with high affinity and with high selectivity, so that there is no need to have two separate domains present, nor to assure that these two domains are present in the right spatial conformation and configuration (i.e. through the use of especially designed linkers, as with scFv's);
V_{HH} domains and Nanobodies can be expressed from a single gene and require no post-translational folding or modifications;
V_{HH} domains and Nanobodies can easily be engineered into multivalent and multispecific formats (as further discussed below);
V_{HH} domains and Nanobodies are highly soluble and do not have a tendency to aggregate (as with the mouse-derived antigen-binding domains described by Ward et al., Nature, Vol. 341, 1989, p. 544, therein also referred to as “single domain antibodies”);
V_{HH} domains and Nanobodies are highly stable to heat, pH, proteases and other denaturing agents or conditions;
V_{HH} domains and Nanobodies are easy and relatively cheap to prepare, even on a scale required for production (e.g. as further described below). For example, V_{HH} domains, Nanobodies and proteins/polypeptides containing the same can be produced using microbial fermentation, and do not require the use of mammalian expression systems, as with for example conventional antibody fragments;
V_{HH} domains and Nanobodies are relatively small compared to conventional 4-chain antibodies and antigen-binding fragments thereof, and therefore show high(er) penetration into tissues (including but not limited to solid tumors) than such conventional 4-chain antibodies and antigen-binding fragments thereof
V_{HH} domains and Nanobodies can show so-called cavity-binding properties, and can therefore also access targets and epitopes not accessible to conventional 4-chain antibodies and antigen-binding fragments thereof. For example, it has been shown that V_{HH} domains and Nanobodies can inhibit enzymes (see for example WO 97/49805; Transue et al., Proteins: structure, function, genetics, 32: 515-522 (1998); Lauwereys et al., EMBO J., Vol. 17, No.13, p. 3512-3520).

In addition to Camelids, heavy chain antibodies also occur naturally in for example certain species of sharks (see for example the International application WO 03/014161). Although variable domains derived from such heavy chain antibodies may be used in the invention, the use of Camelid-derived heavy chain antibodies and/or of the variable domain sequences thereof is much preferred, inter alia because the latter are derived from a species of mammal and/or because the latter will generally be easier to “humanize” (as described below).

As also described in the general background art cited above, the heavy chains of naturally occurring heavy chain antibodies contain CH3 domains, CH2 domains and a variable domain, but—in addition to the light chains—lack the CH1 domains present in the heavy chains of naturally occurring conventional 4-chain antibodies.

Generally, V_{HH} domains have a structure that retains the immunoglobulin fold of conventional V_{H} domains (see for example Desmyter et al., Nature Structural Biology, Vol. 3, 9, 803 (1996); Spinelli et al., Natural Structural Biology (1996); 3, 752-757; Decanniere et al., Structure, Vol. 7, 4, 361 (1999); Dumoulin et al., Protein Science (2002), 11:500-515). However, compared to V_{H} domains, V_{HH} domains contain one or more substitutions in their amino acid sequence (and in particular in their framework regions) that make the region(s)/residues of the V_{HH} domain that in a V_{H} domain would form the V_{H}/V_{L} interphase more hydrophobic (see the general background art cited above and the further discussion below). These substitutions also distinguish V_{HH} domains (as well as Nanobodies) from naturally occurring antigen-binding domains that are derived from conventional 4-chain antibodies, such as the mouse derived V_{H} sequences described by Ward et al., supra.

Generally, for the various uses of V_{HH} sequences (and of Nanobodies based thereon), as well as the advantages that the use of V_{HH} sequences (and of Nanobodies based thereon) can provide in such applications (i.e. compared to V_{H} sequences), reference is again made to the general background art. Other potential applications and uses of V_{HH} sequences and Nanobodies will be clear to the skilled person, and will for example depend on the antigen against which said sequence is directed and other properties for which the V_{HH} sequence or Nanobody has been selected. Generally, however, it can be said that V_{HH} sequences can be used for any application or use for which the use of conventional immunoglobulin sequences (including but not limited to conventional antibodies and fragments thereof, as well as scFv's and V_{H} domains) has been proposed or can be envisaged.

As also generally described in the general background art cited above, naturally occurring V_{HH} domains can be used as Nanobodies™. In addition, as also described below, the amino acid sequences of naturally occurring V_{HH} domains, and/or the nucleic acids and/or nucleotide sequences encoding the same, can be used as a starting point for developing, designing and/or preparing Nanobodies™, e.g. by using one of the various methods outlined in the discussion of the general background art below.

For example, as further described below, one particularly preferred, but non-limiting class of Nanobodies are Nanobodies of which the amino acid sequence, compared to the sequence of a naturally occurring V_{HH} domain, has been “humanized”, i.e. by replacing one or more of the amino acid residues in the amino acid sequence of a naturally occurring V_{HH} domain with the amino acid residue(s) that occur at the corresponding position(s) of a conventional human V_{H} domain.

Another non-limiting class of Nanobodies, also further described below, are Nanobodies of which the amino acid sequence, compared to the sequence of a naturally occurring V_{H} domain, and in particular compared to the sequence of a naturally occurring V_{H} domain from a human being, has been “camelized”, i.e. by replacing one or more of the amino acid residues in the amino acid sequence of a naturally occurring V_{H} domain with one of the “hallmark residues” indicated below. As further described herein, the invention also generally comprises Nanobodies comprising one or more of such camelizing substitutions, irrespective of the way these Nanobodies have been generated or obtained (for example, by camelization, by synthesis de novo or in any other way).

For a more detailed description of Nanobodies, reference is made to the further description below.

Discussion of the General Prior Art on Cloning Immunoglobulin Sequences.

The art describes various methods and techniques for cloning immunoglobulin sequences.

Traditionally, monoclonal antibodies have been generated using the hybridoma technique of Kohler and Milstein. In said method, a B-cell expressing an antibody of interest is fused with a suitable neoplastic cell (or alternatively a T lymphocytes is fused with a lymphoma cell), so as to provide an immortalized hybridoma cell, that can be used to produce the desired antibody.

The use of the hybridoma technique therefore generally requires a cell expressing antibodies against the desired antigen. Such cells can freely be generated by immunizing a non-human mammal with the desired antigen, so as to raise an immune response against said antigen. However, suitable human cells expressing antibodies against the desired antigen for use in the hybridoma technique may not easily available since, for obvious health reasons, it may not be possible to immunize a human being with the intended antigen.

Some of the early work in cloning immunoglobulin sequences of conventional 4-chain antibodies using amplification is described by Larrick et al. (see for example Larrick et al., Biotechnology, Vol. 7, September 1989, p. 934-938; and Larrick et al., Progress in Biotechnology (Borrebaeck et al., Ed.), Vol. 5, p. 231-246 (1989)). In the work of Larrick et al., PCR is used to selectively amplify variable domain sequences starting from individual hybridoma cells and/or individual B-cells, using suitable consensus primers.

Another techniques for amplifying immunoglobulin sequences of conventional 4-chain antibodies from individual B-cells are described in Coronella et al, Nucleic Acids Research, 2000, Vol. 28, No. 20, e85; Takahashi et al., Journal of Biotechnology 49 (1996), 201-210; Embleton et al., Nucleic Acids Research, Vol. 20, No.15, 3831-3837; and the International application WO 92/02551.

One of the most widely used techniques for cloning immunoglobulin sequences comprises a combination of the so-called “repertoire cloning” and “phage display” techniques, as for example described in EP 0 589 877, U.S. Pat. No. 5,969,108 and U.S. Pat. No. 6,248,516. As described in these and other references, the selection and cloning of immunoglobulin sequences (also generally referred to below as “binders”) by means of repertoire cloning and phage display generally involves the steps of:
a) providing a sample of RNA such as total RNA or mRNA from a cell or collection of cells, wherein said cell can express the entire immune “repertoire” from an animal (such as B-cell) and wherein said mRNA contains the entire immune repertoire of said animal;
b) synthesizing cDNA out of said mRNA;
c) selectively amplifying the nucleotide sequences that encode the immune repertoire;
d) preparing phage particles that express the binders encoded by said amplified sequences on their surface; using a suitable micro-organism, such as E. coli:
e) selecting phage particles that express binders sequences that can bind to a desired antigen;
f) isolation/cloning of the binder-encoding sequences from the phage particles selected in step e).

Specific primers for performing the amplification step c) so as to selectively amplify the sequences of variable domains of conventional 4-chain antibodies are described in the European patent EP 0 368 684. According to this patent, the sequences obtained after the amplification step are cloned into an expression vector, so as to provide an expression library comprising a repertoire of nucleic acid sequences for expression of a repertoire of proteins each comprising an immunoglobulin variable domain.

The use of repertoire cloning and phage display has also been described for the cloning of variable domain sequences of heavy chain antibodies (see for example Reiter et al., J. Mol. Biol. (1999), 290, 685-698).

The international application WO 03/54016, as well as some of the applications by Ablynx referred to above, describe improved primers for selectively amplifying variable domain sequences of heavy chain antibodies. Generally, when used for the cloning of variable domain sequences from heavy chain antibodies, and compared to the use of for example the primers that are described in EP 0 368 684, the use of these improved primers allow for the cloning of a more extensive repertoire of variable domain sequences.

Although repertoire cloning and phage display have established themselves as highly successful and efficient techniques for cloning and screening variable domain sequences, there are a few disadvantages associated with the use of these techniques.

First of all, although the selection step e) above to some degree allows for the selection of binders that bind to the desired antigen, in practice—after the isolation step f)—one or more further screening steps against the desired antigen are usually required to provide binders with adequate affinity and specificity. This is because the pool of binders that is obtained after the selection step f) often still contains a lot of “noise”, such as false positives, empty vectors, etc.

Also, in the selection step e), the binders are expressed on the surface of phage particles, and are selected whilst bound to these phage particles. Thus, the binders are expressed and selected under conditions that are very different from the conditions under which these binders are expressed in vivo, i.e. on the surface of B-cells or other antibody-expressing cells. Because of this, it is possible that some binders that are capable of binding an antigen under “natural” conditions, are no longer capable of binding the antigen when they are subjected to the “artificial” conditions presented by phage display, or show a reduced affinity for the antigen under such conditions. Such binders would therefore not be identified as “positives” when phage display is used (for example, one non-limiting reason for this could be that, on a B-cell, a number of binders can “work together” to provide high affinity for the antigen. When phage display is used, such naturally occurring high affinity would be lost). In this respect, it should be noted that there are binders (such as the anti-TNF binders that are described in the International application WO 04/041862 by Ablynx et al.) that show a 10-fold to 1000 fold or more increase in affinity when they are formatted as a bivalent construct, compared to the corresponding monovalent construct.

Also, when the selection step is performed using binders that are expressed on the surface of phage particles, it is not assured that the binders are expressed in the right way to bind to the antigen (i.e. compared to the way the binders are expressed on the surface of B-cells). For example, there may be spatial or steric interactions with the phage particle—which is many hundreds or thousands time bigger than the binder itself—which may hinder or even fully prevent high affinity binding of the binder to the antigen. Although this disadvantage may in some cases be somewhat ameliorated by using a phage particle in which the binder is presented at the end of a sufficiently long “linker” (such as a pIII loop), in general, it can again be said that performing a selection step by means of phage display leads to a strong bias towards binders that can bind with high affinity to the antigen when they are expressed on the surface of a phage particle. This again means that certain binders that are capable of binding with high affinity in the “natural” context of expression by and/or on the surface of a B-cell may not be identified as positives with high affinity under the “artificial” conditions of phage display.

Another disadvantage of phage display is that the phage particles must be prepared in a micro-organism such as E. coli. This means that binders (or sequences encoding them) which are not compatible with the conditions used for preparing the phage particles or not produced well in E. coli (e.g., E. coli bias), and/or cannot take on their natural conformation under these conditions, and/or lose all or part of their affinity for the antigen under these conditions, will not be incorporated in or expressed on the phage particles, and may therefore be missed as positives.

Also, generally, phage display techniques are in practice highly sensitive to contamination.

Another disadvantage of phage display, in particular when it is performed in order to obtain binders derived from conventional four-chain antibodies, is that in general, the “total” mRNA used as the starting material contains a very low percentage of high affinity binders. In addition to this, for a number of reasons, the cloning steps that are performed in order to provide the sequences that are expressed on the phage particles for the selection step also lead to a “dilution” of the number of high affinity binders compared to the total number of binders. This means that large numbers of phage particles (i.e. more than >10⁸ phage particles, often 10¹¹-10¹² phage particles or more) must be used in the selection step. Also, with binders derived from conventional 4-chain antibodies, the phage libraries are often not very functional, since the variable heavy or light chain domain is expressed without its natural binder partner (i.e. its variable light or heavy chain domain, respectively). Thus, with phage display the original pairing of the antibody heavy and light chains, both chains of which contribute to the antibody's binding affinity, is lost in the cloning process, and the probability of restoring these original pairs is extremely low when starting from a population of B lymphocytes from immunized or non-immunized animals or humans. In general, significant manipulations are required to achieve affinity levels appropriate for clinical efficacy.

Thus, generally, the use of phage display has a number of disadvantages that are inherent to the use of phage particles for the expression of the binders in the selection step. Also, any subsequent screening step(s) that are performed on the binders selected using phage display will by definition only lead to binders that can be identified (i.e. because they already have been identified in a foregoing step) as positives using phage display. This means that the format used for phage display—i.e. expression as a monovalent binder on the surface of a phage particle under “artificial” conditions—in practice puts a limitation on the number and type of binders that can be expressed in E. coli and/or that can be identified in the subsequent screening steps, and/or leads to a bias towards binders that show high affinity when present in a monovalent form on the surface of a phage particle.

All this means that, for example, binders that by themselves (i.e. in monovalent form) do not have a particularly high affinity for the desired antigen but do bind with high affinity when present on a B-cell (because they bind as a “multimer”) and/or that are not well expressed in E. coli, can not be easily identified or selected for using phage display. Also, phage display will not allow the easy identification and/or selection of binders that, when formatted as a bivalent or multivalent construct, lead to an “order of magnitudes” increase of affinity compared to the corresponding monovalent binder (such as the anti-TNF binders referred to above).

In this respect, as also mentioned in the general background art cited herein and as further discussed below, V_{HH} domains and Nanobodies based thereon are often formatted for pharmaceutical use as a fusion with other functional amino acid sequences, for example as a multispecific protein containing two or more Nanobodies directed against different antigens. In practice, it has been found by applicant that binders which show a high affinity in monovalent form sometimes lose their affinity or other desired properties to a lesser or larger extent when they are formatted with other amino acid sequences; whereas other Nanobodies which initially did not show as high an affinity in monovalent form actually prove more useful for such formatting. Again, for finding such binders, the bias towards binders that show a high affinity when present in a monovalent form on the surface of a phage particle that is inherent in phage display may be a major disadvantage.

Thus, although conventional display methods have achieved considerable success in isolating antibodies and other polypeptides with specific binding to selected targets, some inefficiencies and limitations remain. In conventional methods, many library members bind nonspecifically to the target or the solid phase bearing the target and are amplified along with specifically bound library members causing poor efficiency at each round of affinity selection. Not only can this waste time and effort in performing many rounds of affinity selection, but members bearing polypeptides having specific affinity are lost at each round. Selection is generally terminated when sufficient rounds of affinity selection have been performed to achieve a significant number of members bearing polypeptides with affinity for a target even though many nonspecifically binding members are still present. Clonal isolates are then picked and tested individually to reduce the risk of losing specific-binding members through further rounds of selection. Clonal isolates shown to bind specifically may then be cloned into an expression work for future analysis, and, large-scale production. Accordingly, only one or a few of library members bearing polypeptides with specific affinity for the target present in the original repertoire are ever isolated.

### BRIEF SUMMARY OF THE INVENTION

It is an object of the invention to provide an alternative method for obtaining immunoglobulin sequences of heavy chain antibodies.

In particular, it is an object of the invention to provide an alternative method for obtaining immunoglobulin sequences of heavy chain antibodies that does not have the disadvantages of repertoire cloning in combination with phage display.

More in particular, it is an object of the present invention to provide an improved method of identifying, selecting, generating and/or cloning nucleic acids and/or nucleotide sequences that code for heavy chain antibodies or antigen-binding fragments thereof (including but not limited to V_{HH} domains).

Even more in particular, it is an object of the present invention to provide an improved method of identifying, selecting, generating and/or cloning nucleic acids and/or nucleotide sequences that code for heavy chain antibodies or antigen-binding fragments thereof (including but not limited to V_{HH} domains), wherein said heavy chain antibodies or antigen-binding fragments thereof are directed against a specific antigen.

It is also an object of the invention to provide such antigen-binding fragments of heavy chain antibodies (in particular including, but not limited to, V_{HH} domains), that can either be used as Nanobodies, or that can be used as a starting point for generating Nanobodies (as further described herein).

Further objects, aspects, embodiments, uses, applications and advantages of the invention will become clear from the further description hereinbelow.

Generally speaking, the method of the invention takes advantage from the fact that a sample or population of cells that is enriched in cells expressing, or capable of expressing, a heavy chain antibody against a desired antigen is readily available as a starting material for generating the desired immunoglobulin sequences, i.e. by immunizing a species of Camelid with the desired antigen, using a suitable regimen (for which generally, reference is made to the general background art cited above). The invention uses such an enriched sample or population as a starting material to provide individual cells expressing a heavy chain antibody against the desired antigen, from which in subsequent steps immunoglobulin sequences against said antigen can be obtained. As mentioned below, said immunoglobulin sequences can be full-chain antibodies, single chains thereof or antigen-binding fragments thereof, or nucleotide sequences/nucleic acids encoding the same. According to one preferred, but non-limiting embodiment, the methods of the invention are used to provide V_{HH} domains or nucleotide sequence/nucleic acids encoding the same.

To better understand the present invention, in particular in relation to conventional repertoire cloning and phage display techniques in the way that these techniques are commonly applied to cloning human V_{H} and V_{L} sequences, it should again be noted that usually, it is not possible to provide a comparably enriched population of cells from a human source (i.e. as a starting material for generating human V_{H} and V_{L} sequences), simply because it is not feasible for health reasons to immunize a human being with the desired antigen.

Thus, when human V_{H} or V_{L} sequences are to be generated, usually a naïve non-enriched population of cells is used as a starting material. Because this starting material has not been enriched, it contains a much lower percentage of binders that recognize the desired antigen (i.e. compared to an enriched population). This in turn means that a much larger number of V_{H} sequences and V_{L} sequences (i.e. in the region of 10⁸-10¹² sequences) needs to be screened in order to identify a suitable binder. In current practice, the screening of such large libraries can only be performed efficiently using phage display techniques.

In addition, when phage display is used, V_{H} sequences and V_{L} sequences can only be screened separately for interaction with the antigen. This means that after V_{H} domains and V_{L} domains that recognize the antigen have been identified, it must still be determined whether the identified domains can actually interact together to jointly bind the relevant antigen with sufficient affinity and specificity. In practice, a lot of V_{H} domains and V_{L} domains that are positive for interaction with the antigen during the step of phage display do not lead to a suitable V_{H}/V_{L} combination. This leads to a large “loss” of “positives” following phage display, which in turn means that even more domains need to be screened in order to identify suitable binders (of course, with V_{HH} domains, this problem does not arise, since V_{HH} domains do not require a light chain variable domain in order to bind the antigen with sufficient affinity and specificity).

The present methods avoid these disadvantages inherent to both the use of a non-enriched starting material as well as to the use of repertoire cloning and phage display as normally associated with the use of such non-enriched starting materials.

Thus, according to a first aspect, the invention relates to a method for generating or cloning a nucleic acid or nucleotide sequence that encodes a heavy chain antibody or an antigen-binding fragment thereof, wherein said heavy chain antibody or antigen-binding fragment is directed against a specific antigen, said method comprising the steps of:
a) providing a sample or population of cells from a Camelid immunized with said antigen, or population of cells from a non-immune Camelid immunized in vitro with said antigen, wherein said sample or population of cells comprises at least one cell that expresses or is capable of expressing a heavy chain antibody directed against said antigen;
b) isolating from said sample or population said at least one cell that expresses or is capable of expressing a heavy chain antibody directed against said antigen;
c) obtaining from said at least one cell a nucleic acid or nucleotide sequence that encodes a heavy chain antibody directed against antigen or that encodes an antigen-binding fragment thereof directed against said antigen.

For the sake of simplicity, in the further description below, the different steps of the methods of the invention will be described in more detail with reference to the cloning, screening and use of V_{HH} sequences, which is a preferred but non-limiting embodiment of the invention. It will however be clear to the skilled person that the methods described herein can be applied analogously to any other immunoglobulin sequence derived from a heavy chain antibody.

The sample or population of cells used in step a) may be any suitable sample or population of cells that comprises at least one cell that expresses or is capable of expressing a heavy chain antibody directed against said antigen. For example, said sample or population of cells may be a sample of primary blood lymphocytes, lymph node cells or other B-cells, or spleen cells.

Said sample or population may be obtained from a Camelid that has been suitably immunized with said antigen (i.e. so as to invoke an immune response against said antigen). Any suitable method or regimen known per se for immunizing a Camelid with the antigen may be used, for which reference is made to the general background art cited above.

After such suitable immunization, the sample or population of cells may be obtained from the Camelid in a manner known per se, for which again reference is made to the general background art cited above. For example, the sample or population of cells may be collected in the form of a sample of whole blood, or in the form of a fraction of whole blood that contains cells expressing heavy chain antibodies, such as a serum sample, in the form of lymph fluid or in the form of a tissue sample such as a sample of spleen cells. To obtain such a fraction of whole blood, first a sample of whole blood may be obtained, from which then a suitable fraction can be obtained in a manner known per se.

Said sample or population may also be obtained from a naive Camelid, using the sampling and isolation methods as described above, after which the lymphocytes are immunized in vitro by culturing the cells ex vivo and exposing them once or repeatedly to said antigen. Cells may be provided with additional growth factors, antigen presenting cells, adjuvants, antiCD40 antibodies, CD40L or combinations thereof to optimize in vitro activation and/or differentiation of antigen specific B-cells, as will be clear to the skilled person.

It is also possible to subject said whole blood or fraction to cell-sorting techniques known per se, to further separate the cells capable of expressing antibodies/heavy chain antibodies. For example, techniques known per se for obtaining a sample or population of B-cells from whole blood, a fraction of whole blood or from lymph fluid can be used to obtain a sample or population of B-cells.

Next, a single cell that expresses or is capable of expressing a heavy chain antibody directed against said antigen is isolated from said sample or population. This may again be performed in any suitable manner known per se. Suitable techniques will be clear to the skilled person, and will usually involve the use of an antigen, an immobilized antigen and/or a suitably marked antigen to select cells expressing a heavy chain antibody directed against said antigen.

Suitable techniques for example include, but are not limited to, contacting the cells with a suitably marked antigen, such as a fluorescently labeled or magnetically labeled antigen, and then subjecting the cells to a separation technique in which the cells that bind the fluorescently labeled antigen are separated from the cells that do not bind said antigen. This may be performed in any suitable manner known per se, such as with a FACS apparatus or another suitable cell sorter. Suitable labels and methods for preparing labeled antigens will also be clear to the skilled person. The cells that bind the fluorescently labeled antigen are then collected, optionally separated from the fluorescently labeled antigen, and optionally separated into individual cells. This may be again performed in a manner known per se, as will be clear to the skilled person.

Other techniques may involve the use of a surface or carrier on or to which the antigen is bound. The cells that do not bind to the antigen/carrier are then washed away, upon which the cells that bind to the carrier or surface are released from the carrier or surface, collected, and optionally separated into individual cells. Alternatively, the carrier with the cells attached to it may be separated from the medium, after which the cells that bind to the carrier or surface are released from the carrier or surface, collected, and optionally separated into individual cells.

In case small particulate carriers such as magnetic microbeads are used, the carriers may be left attached to the carrier binding B-cells after separation of carrier binding and non-binding B-cells.

Suitable carriers and techniques will be clear to the skilled person, and for example include panning with a surface coated with the antigen, the use of a polymeric matrix or gel to which the antigen is attached (i.e. covalently or otherwise), or the use of beads coated with the antigen or to which the antigen is attached (i.e. covalently or otherwise), such as Dynabeads™, MACS beads or other types of magnetic beads. The antigen may also be bound to and/or present on a suitable membrane, including but not limited to a cell membrane or cell membrane fraction.

In case detachment of carrier- or surface-bound B-cell is called for, bound B-cells may be removed from the carrier or surface by enzymatic treatment such as trypsin or other proteases, addition of bivalent cation chelating agents such as EDTA to the medium, addition of agents breaking down the physical link between antigen and carrier or surface such as DTT when reduceable linkers were used, competitive displacement with another antigen binding ligand, or combinations thereof.

For other techniques, reference is for example made to the techniques described in the references mentioned above.

In this respect, it should be noted that a sample or population of antibody-expressing cells obtained from a Camelid will usually comprise cells that express heavy chain antibodies (e.g., in the region of 1-60%, usually between 10 and 30% of all antibody expressing cells), as well as cells that express conventional 4-chain antibodies. For camels, about 50% of all antibody expressing cells express heavy chain antibodies. For llamas, about 30% of all antibody expressing cells express heavy chain antibodies.

Thus, preferably, this aforementioned separation of the cells that express the heavy chain antibody against the desired antigen is most preferably performed in such a way that only cells are obtained that express heavy chain antibodies against the desired antigen, and not cells that express conventional 4-chain antibodies against the desired antigen. For this purpose, before, during or after the selection with the desired antigen, the cells may be subjected to a step in which cells that express heavy chain antibodies are separated from cells that express conventional 4-chain antibodies. Suitable techniques will be clear to the skilled person, and may for example involve the use of antibodies specifically directed against heavy chain antibodies, which may again be either suitably labeled or attached to a suitable carrier or surface. Suitable techniques will be clear to the skilled person, and may be analogous to the techniques described in the paragraphs hereinabove.

Thus, in the above embodiment, step b) may comprise any suitable combination of the following steps:
b-1) separating cells that express antibodies from cells that do not express antibodies;
b-2) separating cells that express antibodies against the desired antigen from cells that express antibodies directed against other antigens;
b-3) separating cells that express heavy chain antibodies from cells that express conventional 4-chain antibodies;
   in which said steps may be performed in any order and in which each two or all three of said steps may also be performed as a single step.

Alternatively, the sample or population of cells may be separated into individual cells expressing a heavy chain antibody against the desired antigen by means of a limiting dilution assay or a similar technique.

According to yet another embodiment, activated cells that express heavy chain antibodies, and in particular activated plasma cells/B-cells that express heavy chain antibodies, are separated from the other cells in the sample (i.e. cells that do not express antibodies, cells that express conventional 4-chain antibodies, and non-activated B-cells that express heavy chain antibodies), collected as and/or separated into single cells, and then used in the further steps described below. One advantage of this embodiment is that such activated cells generally contain much higher levels of mRNA for the heavy chain antibodies—and also may produce higher levels of heavy chain antibodies—compared to non-activated or “memory” B-cells. Such activated cells may be separated from the other cells in the sample using any technique known per se, including but not limited to cell sorting techniques. A non-limiting example of such a method is described in Example 21. Generally, this method comprises, prior to the cell sorting step, a first staining step using a first labelled antibody against heavy chain antibodies, a step of fixing and permeabilizing the cells, and a second staining step using a second labelled antibody against heavy chain antibodies. Following sorting of the cell sample thus obtained, this allows the activated cells to be obtained as a separate population (see again Example 21).

After the cells that express heavy chain antibodies against the desired antigen have been either collected as single cells, or collected and then separated into single cells, a nucleic acid encoding said heavy chain antibody or an antigen-binding part thereof can be obtained from said individual cell. This can again be performed in a manner known per se, as will be clear to the skilled person. Suitable techniques will usually involve an amplification step using suitable primers (e.g. using PCR or another suitable amplification techniques, and using as a template cDNA generated from mRNA obtained from the individual cell(s)), followed by isolation of the amplified products. Reference is made to the prior art and the handbooks referred to above.

For example, for the amplification of variable domain sequences (i.e. V_{HH} sequences), some of the primers known per se for the amplification of heavy chain variable domain sequences can be used, for which reference is made to the prior art cited above. Some particularly preferred primers for the amplification of V_{HH} sequences are the primers referred to in the international application WO 03/54016, as well as some of primers described in the other patent applications by Ablynx et al. mentioned above.

Suitable conditions and reagents for performing said amplification, as well as suitable techniques for isolating the amplified sequences, will also be clear to the skilled person. Reference is again made to the prior art and the handbooks referred to above.

After the amplification step, the nucleic acid thus obtained may be sequenced and/or used to express the V_{HH} fragments, for which reference is made to the further description below.

According to one non-limiting embodiment, prior to the amplification, the individual cells may be cultivated, for example under conditions such that the individual cells can divide/multiply/propagate, and/or under conditions such that the cells are stimulated to express or produce the desired antibody. Suitable methods and techniques will be clear to the skilled person. For example, the cells may be cultivated in a suitable medium in the wells of a multi-well plate. Suitable techniques for stimulating the production or expression of antibodies will also be clear to the skilled person, and may for example include stimulation with suitable Camelid cells (e.g., helper cells), EL4-B5 cells (see for example Weber et al., Journal of Immunological Methods 278 (2003) 249-259), CD40 ligand or a similar factor, or membrane bound CD40 ligand (see for example U.S. Pat. No. 6,297,052 and the further references discussed therein).

Also, in this step, the medium or supernatants in which the cells are cultivated may be screened for the presence, the expression and/or the production of a suitable heavy chain antibody against the desired antigen.

In an alternative embodiment, the method of the invention may comprise the steps of:
a) providing a sample or population of cells from a Camelid immunized with said antigen, wherein said sample or population of cells comprises at least one cell that expresses or is capable of expressing a heavy chain antibody directed against said antigen;
b) isolating from said sample or population, as at least one individual cell or as a set of individual cells, cells that express heavy chain antibodies;
c) screening said at least one individual cell or set of individual cells for the expression of a heavy chain antibody directed against said antigen;
d) obtaining from said at least one cell a nucleic acid or nucleotide sequence that encodes a heavy chain antibody directed against antigen or that encodes an antigen-binding fragment thereof directed against said antigen.

In this embodiment, step a) may be performed in the manner described above. In step b), the cells are first separated into cells that express heavy chain antibodies on the one hand, and cells that either do not express antibodies or cells that express conventional 4-chain antibodies on the other hand. This may be performed as indicated above.

The cells that express the heavy chain antibodies are then separated into individual cells, upon which each individual cell is then screened for the expression of a heavy chain antibody against the desired antigen, for example using one of the techniques described hereinabove. Alternatively, in this embodiment, the individual cells may be cultivated and/or stimulated to express or produce the desired antibody (again essentially as described above), after which the medium or supernatant from each individual cell is screened for the presence of a heavy chain antibody against the desired antigen. The latter avoids having to screen individual cells for the expression of an antibody.

Then, a nucleic acid encoding said heavy chain antibody or an antigen-binding fragment thereof (e.g. a V_{HH} domain) is obtained from the one or more individual cells that express or produce the desired antigen, essentially as described above.

In yet another embodiment, the method of the invention comprises the steps of:
a) providing a sample or population of cells from a Camelid immunized with said antigen, wherein said sample or population of cells comprises at least one cell that expresses or is capable of expressing a heavy chain antibody directed against said antigen;
b) separating said sample or population of cells into a set of individual cells;
c) screening set of individual cells for cells that express of a heavy chain antibody directed against said antigen;
d) obtaining, from said at least one cell that expresses a heavy chain antibody directed against said antigen, a nucleic acid or nucleotide sequence that encodes a heavy chain antibody directed against antigen or that encodes an antigen-binding fragment thereof directed against said antigen.

In this embodiment, the sample or population of cells is first separated into individual cells. Each individual cell is then screened for expression of a heavy chain antibody directed against the desired antigen, essentially as described hereinabove.

Alternatively, in this embodiment, the individual cells may be cultivated and/or stimulated to express or produce antibodies (again essentially as described above), after which the medium or supernatant from each individual cell is screened for the presence of a heavy chain antibody against the desired antigen. The latter again avoids having to screen individual cells for the expression of an antibody.

Then, a nucleic acid encoding said heavy chain antibody or an antigen-binding fragment thereof (e.g. a V_{HH} domain) is obtained from the one or more individual cells that express or produce the desired antigen, essentially as described above.

It will be clear to the skilled person that the above methods can be adapted for use in an automated, medium to high throughput format, for example using suitable robotics. For this purpose, individual cells can also be cultivated in the wells of a suitable multi-well plate.

Individual B-lymphocytes carry randomly rearranged immunoglobulin genes, which impart different antigen specificities to the corresponding protein produced by the various B-cell clones. As rearrangements arise in all clones, and large numbers of B-cells are continuously produced throughout life, the relative abundance of any given rearrangement versus the total number of B-cells is very low.

By immunization or through natural immunity to previous infections, animals convert from an immunologically naive animal state to an immunized state. During this process, B-cell clones reacting to the antigen in question are “activated” and undergo a massive expansion in numbers whereas non-reactive cells do not, leading to a higher relative abundance of the antigen-reactive B-cells in the whole population. Concurrently, affinity maturation occurs, whereby antigen-reactive B-cells undergo cycles of mutations in the immunoglobulin gene and selection of the higher affinity variants resulting from these mutations. Thus, immunization leads to both an increased relative abundance of antigen-reactive B-cells, as well as to higher affinity antibody producing B-cells.

Thus, according to one preferred embodiment of the present invention, the cell-population is obtained from hyperimmunized animals where the antigen-specific B-cell population has expanded to great numbers ( 1/100 or more, orders of magnitude greater than 1/10,000), all of which have evolved high affinity receptors to the antigen in question. Due to the unique structure of the heavy chain antibodies, only isolation of the gene for the heavy chain variable domain is required, without the need for isolating or cloning light chain variable domain. For the reasons indicated above, this is a major advantage over techniques based on conventional 4-chain antibodies from non-immunized animals, and in particular from human beings, for which large scale, high throughput techniques such as the use of repertoire cloning and phage display are generally required to recapitulate the entire antigen specific B-cell repertoire when using samples obtained from unimmunized (or weakly immune) subjects, such as healthy people. In the latter case, most often, the original B-cell population has had no exposure to the antigen of interest. Thus, only very few cells are reactive to the antigen in question and these have not undergone affinity maturation. Isolating these from the total repertoire is not practicable without the most comprehensive and sensitive tools. To make matters worse, with variable domains from conventional 4-chain antibodies, a perfect match of the variable domains is required to bind the antigen with any appreciable affinity. Separately amplifying both highly diverse repertoires and recombining them randomly (to create scFv fragments or Fab fragments) amplifies the problem.

To clarify the numerical scales involved: say 1/10,000 B-cells is of interest, where all B-cells contain heavy chain and light chain genes of interest (“H” and “L”, respectively). 1/10,000 gene of interest “H” frequency× 1/10,000 gene of interest “L” frequency results in 1 “H/L” combination of interest per 100,000,000 recombinations possible. Herein, this problem will be referred to as the “recombination dilution penalty”. For technical reasons, even the right combination of “H/L” gene fragments may fail to bind the antigen with the same affinity as the original full-length combination. Thus, in order to achieve for example scFv production from unimmunized animals/people, it is necessary to recapitulate the entire repertoire present to capture the very few productive receptors. In this respect, it should also be kept in mind that, even if there are some “H” or “L” genes present which are capable of binding the antigen by themselves (and these would be extremely limited in number compared to the “H” and “L” genes that cannot sufficiently bind the antigen by themselves), such genes did not evolve for high affinity binding in isolation of each other—thus, most “H” or “L” genes at best present at 1/10,000 frequencies will not function at all. The fraction that will bind the antigen without requiring pairing will be quite low, again reducing the 1/10,000 hit rate by orders of magnitude. This high proportion of non-functional genes constitutes, in effect, a second independent efficiency reducing penalty. Thus, the overall hit rate will be much lower than the 1/10,000 one might expect based on the 1/10,000 “H” or “L” gene frequency but not knowing most “H” or “L” genes isolated thus will be non-functional. As in the procedure for scFv isolation, for example, this process therefore absolutely requires immense throughput and total repertoire recapitulation to get any high affinity binders at the end of the day.

As mentioned above, the present invention starts from hyperimmunized animals where the antigen-specific B-cell population has expanded to great numbers ( 1/100 or more, orders of magnitude greater than 1/10,000), all of which have evolved high affinity receptors to the antigen in question. Due to the unique structure of the receptor, only isolation of the “H” gene is required. Thus, it is possible to obtain “H” genes encoding high affinity antigen binding receptors without needing to isolate “L” genes, let along dilute the combinations of interest by making random “H/L” combinations—avoiding the “recombination dilution penalty” entirely. Furthermore, all “H” genes have evolved to bind antigen without requiring the “L” gene, thus the second efficiency penalty type also does not apply to the present invention. This unique combination of A: the use of immunized animals and B: lack of efficiency penalties intrinsic to the heavy-chain antibody class enables one to get binders even by screening a relatively low number of randomly picked B-cell clones. In this respect, reference is also made to Table 1 in Frenken et al. (J. Biotech., 2000, 78: 11).

Thus, in the invention, neither massive throughput nor absolute total repertoire recapitulation (that are essential when using repertoire cloning and phage display starting from B-cells from human beings) are necessary given the very low requirements to high affinity binder isolation, intrinsic to hyperimmune animals on the one hand and especially camelid immune function in particular.

Finally, although the present invention in its most preferred embodiment provides its major advantage when used with a population of cells that comprises cells expressing heavy chain antibodies against the desired antigen, it is not excluded to apply the invention to other samples of conventional antibody-producing cells. For example, a transgenic non-human animal that expresses human or human-like conventional 4-chain antibodies against a desired antigen (i.e. raised through suitable administration) can be used as a source of B-cells that can be used as a starting material for the methods of the present invention, optionally after enrichment for the desired antigen-expressing cells. One non-limiting example of such a transgenic animal is the XenoMouse™ of Abgenix, Calif., USA, a transgenic mouse that expresses human antibodies upon immunization with an antigen. Thus, a sample of antibody-expressing cells obtained from a XenoMouse™ can be used as a starting material for use in the methods of the invention, optionally after suitable enrichment for cells expressing the antibodies against the desired antigen.

Activated B-cells produce and secrete large amounts of soluble immunoglobulin, but both resting and activated B-cells also display the immunoglobulin on their cell membrane. Thus, detection of antigen binding to the B-cell surface displayed immunoglobulin identifies the antigen-binding B-cell subpopulation in a mixed antigen-binding/antigen-non-binding B-cell population, such as can readily be isolated from peripheral blood, spleen, lymph nodes, etc. Flow cytometry instruments are exquisitely suited for this purpose, as these are designed to automatically detect and quantitate binding of fluorescently labeled molecules to very large numbers of individual cells. Furthermore, their ability to detect multiple fluorescent labels per cell as well as cell morphology derived parameters enables the user to include various negative controls to exclude binding to irrelevant cells in the sample (such as polymorphonuclear cells, macrophages, T-cells, or dead cells), as well as positive markers (such as markers identifying all B-cells in the sample). Finally, modern flow cytometric sorter devices offer the user the option to physically sort individual cells based on any arbitrarily user-chosen combination of such parameters and dispense them all together into either one bulk collection vessels (such as a centrifuge tube) or one by one into many individual vessels (such as the wells from a microtiter plate). A number of alternative approaches exist as well, whereby one immobilizes the antigen of interest to a solid support or magnetic particle (using absorption, covalent binding, fusion to affinity purification tags) and uses washing steps or a magnet to rinse away unbound cells. However, none of these methods compares favourably with flow cytometry as only one to two-parameter separations can be performed effectively.

Thus, according to the invention, by combining immunization with flow cytometric sorting of the relevant fraction, a higher initial abundance of interesting cells is obtained, increase the affinity of these as well as identify and isolate them for further characterization.

Immunization can be performed using either purified antigen, crude protein mixtures of antigens, peptides representing a specific region of interest on a large protein or conjugates of such peptides to immunogenic carrier proteins, whole cells expressing the antigen of interest or membrane fractions of the latter. Clearly, much experimental flexibility exists here and there are few limitations to the physical nature of the antigen being used. Isolation of the antigen-reactive B-cells, however, does impose strict requirements. For instance, classical feasibility studies in this area focused on experiments where an essentially unlimited supply of pure protein with excellent solubility was available, using proteins which also easily withstood the labelling reaction (biotinylation, fluorescein labelling, etc.). This enabled the experimenters to avoid expanding the number of B-cells reacting to irrelevant “passengers” antigens (such as those occurring in crude protein preparations, whole cells or membrane fractions), but also to isolate the antigen binding B-cell fraction by panning on the same pure antigen immobilized on plates, magnetically isolating B-cells binding biotinylated antigen or sorting cells binding fluorescein-labeled pure antigen.

Many antigens that are suitable targets for therapy of diseased states, including monoclonal antibody therapy, exist in membrane-bound form. Many of these antigens display only a limited portion of the molecule to the outside, where it is available for binding by a monoclonal antibody drug. Importantly, the conformation of many such proteins critically depends on its close association with the cell membrane and/or subdomains of proteins (including itself) embedded into the membrane. The strong hydrophobicity of these molecules makes it impractical (if not impossible) to purify it to homogeneity and chemically label or fuse to an affinity purification tag without fundamentally altering the 3D structure. As isolating B-cells reacting to the native conformation of the membrane protein is desirable for their use as the basis for the creation of monoclonal antibody drugs, this problem represents a significant technological hurdle, currently unmet. The invention includes an easily implemented method for identification and purification of B-cells recognizing such membrane-bound proteins.

For example, a first non-limiting aspect entails the use of a matched pair of a mock-transfected cell line (or primary cell type) not expressing a given membrane-bound protein and a “sibling” cell line derived from the same parental cell line (or primary cell type) as the former, transfected with an expression vector encoding the native, full-length cDNA encoding the membrane-bound protein. As such, this requires no more knowledge of the target protein's structure than the encoding nucleotide sequence. Also, such matched pairs of cell lines can readily be obtained commercially from several suppliers of membrane-bound protein research tools. Expression of the full-length gene in its native environment (i.e. embedded in the cell membrane) guarantees its conformation will match that of the protein expressed by the cells naturally expressing it. Alternatively, one can use the natively expressing cell type and generate a matching non-expressor cell by disrupting the gene using classical “gene knock-out” genetic manipulation tools, or by introducing RNAi strongly suppressing expression of the protein of interest.

Once a matched cell line pair has been obtained as described above, one can label these using fluorescent dyes without any alteration to the extracellular membrane-bound proteins. This can be performed by simple incubation of live cells with a membrane-permeant ester derivative of a chemically activated fluorochrome. These apolar molecules migrate across the cell membrane of live cells into the cytoplasm, where a variety of enzymes with esterase activity hydrolyse the apolar molecule into two highly charged (i.e. polar) fragments. These can no longer cross the membrane, thereby effectively trapping the fluorescent dye in the cell (Molecular Probes, probes.com/handbook/sections/1402.html). Furthermore, careful engineering of dye derivatives has resulted in molecules where one of the two resulting fragments is chemically reactive and spontaneously covalently binds nearby proteins immediately after esterase-mediated hydrolysis. A short incubation of live cells with low concentrations of these dyes results in intense and highly stable fluorescent labelling of the cells, without appreciable loss of viability or altering normal cell function as only a very small portion of intracellular proteins gets modified. An early example of these is the succinimidyl ester of 5-, 6-carboxyfluorescein diacetate (CFSE for short), where the final product is brightly green fluorescent. Many other dyes have been developed since, given rise to a panel of readily available reagents whereby one can pick which color to label a cell with. Some of these dyes have fluorescence spectra widely diverging from CFSE and the like, such as DDAO-SE, thereby minimizing potential colour overlap and enabling multicolour labelling experiments (Molecular Probes, probes.com/lit/bioprobes44/7.pdf).

Therefore, according to this non-limiting aspect, a pair transfected and control cell lines labeled with two fluorescent dyes in the manner described herein that can easily be discriminated, without chemically modifying extracellular proteins, is used.

Any suitable animal (and in particular mammal) can then be immunized with the (unlabeled) transfected cell line, or crude membrane protein extracts thereof. This can be performed in a suitable manner known per se that leads to the generation of an immune response (i.e. antibodies) against the antigen. Due to the complex nature of the immunogen, B-cells reacting with high affinity to both the transgene protein as well as normal membrane proteins of the injected immunogen cell line will be induced. By labelling the non-transgenic and transgenic cell lines in vitro with two different fluorescent dyes as described above, and bringing these into contact with the isolated B-cells from the immunized animal, the antigen reactivity of the B-cells will be easy to determine using flow cytometry, for example as follows (for which reference is also made to FIGS. 22-25):
B-cells with irrelevant antigen specificities will not bind either fluorescently labeled cell population, resulting in a double-negative “bottom-left quadrant” population in bivariate FACS plots
B-cells reactive to membrane-bound proteins naturally occurring on the membrane of the non-transfected (parental) cell line will bind both transfected and non-transfected cells equally, resulting in a diagonal staining patterns in bivariate FACS plots
The B-cells of interest, namely those binding the membrane protein encoded by the transgene cell line only, will bind only the transfected cell line, resulting in a single-positive population close to the X- or Y-axis in FACS bivariate plots. As these can now be easily distinguished from irrelevant B-cells, this population can now be identified and therefore also physically isolated (sorted) away from the rest.

It will be clear to the skilled person in the field of flow cytometry that several variations to this general scheme are possible, without leaving the general concept described herein. For instance, contaminating macrophages or polymorphonuclear cells in the B-cell preparation possibly interacting non-specifically with the fluorescent cells can be gated out based on their different light scatter properties. In the unlikely event T-lymphocytes, sharing the same light scatter characteristics of B-cells, bind the target cells as well, these can readily be identified based on fluorescently identified expression of T-cell markers (positive identification) or lack of co-expression of B-cell markers on fluorescent cell binding particles (negative identification). Antigen binding but dead B-cells, largely unsuitable for downstream in vitro characterization, can be excluded based on non-membrane permeable DNA binding dye such as propidium iodide (PI) or TOPRO-3 (Molecular Probes). It can therefore seem to the casual observer a large number of distinct fluorochromes would be required for this type of experiment. However, as light scatter does not require staining the cells, and several parameters would be used to negatively select dead, non-B-cell or irrelevant antigen-binding B-cells, these can all be combined in the same fluorescence detection channel at the relatively small cost of losing information on the relative abundance of cells not of interest to the researcher for these various reasons (“dump channel”). Indeed, this information can easily be re-obtained by serially analysing but not sorting several small aliquots (input tubes) of the same B-cell preparation, each stained using only one of the reagents used in the multiplex tube used to sort the cells of interest.

Also, it has been previously described that member species of the camelid family of mammals have the capacity to produce both conventional four-chain (2 heavy, 2 light) antibodies as well as non-conventional heavy-chain-only yet functional antibodies. In the context of the present invention, it has been demonstrated using flow cytometry tools that a distinct subset of B-cells isolated from the peripheral circulation of lama glama can be shown to display surface immunoglobulins but no light chains. As both B-cell compartments can differentiate into high-affinity antigen-binding immunoglobulin plasma cells, but only the non-conventional subpopulation is relevant for the production of heavy-chain only antibody derived Nanobodies™, avoiding co-sorting these antigen-binding B-cells together with non-conventional antibody producing B-cells would be highly advantageous. According to one aspect of the invention, two or more flow cytometry-compatible staining reagents can be used simultaneously or consecutively to segregate these two population. Using positive staining for surface light chains as an additional negative sorting decision parameter, not unlike the dead B-cell identification described above, will result in a workable sorting decision matrix whereby only non-conventional antigen-binding B-cells can be sorted from the peripheral compartment of immunized llamas.

Another variation to the general B-cell staining protocol described above concerns the nature of the antigen-specific B-cell staining entity. For reasons of clarity, the B-cell staining procedure described above has been simplified to describe using the fluorescently labeled cells directly as staining reagents. As even activated B-lymphocytes are relatively small, only a limited surface area is available per cell to interact with other cells, such as transfected and non-transfected cells. Steric hindrance between two fluorescent B-cell binding cells could therefore result in unwanted competition and, ultimately, in only one or few target cells binding the individual B-cells. This would not be a major issue if it were not for the case where B-cells react to normal membrane proteins expressed on the non-transfected target cell. Here, it is possible that due to random stochastic effects a number of B-cells would be sorted as transgene-specific (i.e. binding a single or few cells, coincidentally all transgenic and thus similarly single-color fluorescently labeled), whereas they bind antigens not of interest but also present on the transfectant's parental cell line. Co-sorting these cells together with the genuinely transgene-specific B-cells would results in increased “noise” in downstream processing, necessitating processing many more sorted clones in parallel, as well as requiring extensively screening of the resultant Nanobodies™ for binding to transfected and non-transfected cells. Clearly, any increase in upfront B-cell selection will be reflected directly in downstream workload and cost per candidate lead molecule generated. Therefore, the following alternative to using whole cells for B-cell staining was developed.

For example, Cohen et al. described a simple procedure (J. Biol. Chem., 1981, 257: 1523-1531) whereby the A431 cell line could be induced to shed intact anuclear membrane vesicles into the medium by simple incubation in a hypotonic buffer. These vesicles can then be harvested from the medium by a simple centrifugation step and used as inert substitutes for living cells. The lack of contact-induced adhesion such as can occur with living adherent cells, as well the resilience to prolonged freezing without cryoprotecting agents, incubation at 4° C. or even room temperature in various buffers etc. means these vesicles can be used as more convenient substitutes for cells in many in vitro membrane protein binding studies. The “yeast budding”-like mechanism of vesicle formation ensures the extracellular facing membrane side remains facing out, whereas many artificial membrane protein extraction methods reconstitution or artificial liposome generation procedures result in inside-out dominated population or, at best, 50/50 right side out vesicles. Also, in contrast to liposome production methods, no purified or potentially denatured extracted proteins need to be used, and no foreign lipids (potentially resulting in increased background binding) need to be introduced.

As the shed vesicles contain the complete natural cytoplasmic protein content of the original cells, fluorescent vesicles can readily be generated by labelling the cells with membrane-permeable reactive dyes, as discussed previously, prior to induction of hypotonic shock. By labelling mock-transfected and membrane protein of interest-transfected cells using different fluorescent dyes, fluorescent vesicles displaying the normal complement of proteins plus transgenic membrane protein or normal proteins only can be generated. These vesicles are expected to be useful for extremely sensitive detection of rare binding events, and can for example be handled and applied in a manner that is analogous to the handling and use of the fluorochrome-loaded artificial liposomes described in Scheffold et al. (Nat Med, 2000, 6: 107-110), where antibody rather than antigen was bound to the liposome surface for use in detection of membrane-bound molecules occurring at very low numbers per cell. Liposome staining molecules occurring at rates as rare as 80 molecules per cell resulted in easily discriminatable positively staining populations, which cannot be obtained through conventional means. Indeed, fluorescence intensity was demonstrated to be up to 2 logs more intense for the number of binding events per cell expected to occur between a B-cell and an antigen-bearing vesicle, based on the number of immunoglobulins known to be expressed on the surface of each individual B-cell (order of magnitude ˜10⁴/cell).

The shed vesicles, however, generally have diameters in the same order of magnitude as the original cells. Thus, in terms of usefulness in B-cell staining experiments, nothing much appears to be gained from this procedure. However, the structural flexibility of the vesicles makes it possible to repeatedly extrude these through track-etched membranes of defined pore sizes smaller than the vesicle diameter. This procedure has been described per se in the art for the reduction of large multilamellar or unilamellar liposomes with varying but large diameters to homogenous populations of unilamellar liposomes of a diameter roughly similar to the membrane pore size, and is now used according to the invention for producing the vesicles described herein, using the tools and means known per se for reducing the mean size as well as narrow the size distribution of fluorescently labeled large cell-shed vesicles.

Due to their reduced size, many more vesicles will be able to simultaneously access the individual B-cell's cell membrane, thereby dramatically decreasing the chances of erroneously deciding a given cell is transgene specific, based on the absence of binding to the same cell of fluorescent vesicles derived from the non-transfected cell line. Vesicle disruption can be minimized using techniques already optimized for liposome extrusion, such as stepping down gradually from large to small pore size membranes rather than forcing very large liposomes through very small pore size filters, take into account the use of elevated temperatures can increase efficiency etc. Loss of vesicle membrane integrity can easily be monitored by spinning down extruded vesicles using ultracentrifugation and measuring if fluorochromes have been released into the supernatant buffer during the process.

Pick et al. recently described (J Am Chem Soc (2005) 127: 2908-2912) an alternative protocol possibly bypassing the need for extrusion to generate small vesicles, using a combination of cytochalasin treatment and mechanical agitation of the cell cultures. As this is a single-step procedure, requiring fewer handling steps and not involving potentially stressing vesicles to the point of leakage during extrusion, a comparison of the vesicles resulting from this procedure to extruded macrovesicles generated as described above can be made. The overall experimental philosophy, as well as methods for cell labeling prior to vesiculation remain identical to those described above.

Detection and sorting of cells in general poses significant hurdles if the target population is rare. Although this is not expected to be the case for animals immunized with a sufficiently immunogenic target, pre-FACS sort enrichment of the target population may be desirable anyway. One way to go about this is to magnetically label membrane vesicles of the transgenic cell line and enriching the pre-sort sample for the target population using a magnet, or magnetic column. However, as should be obvious from the bivariate FACS plot pictured below (FIG. 25), this strategy will also enrich for B-cells binding the parental cell line. Thus, the relative abundance of irrelevant B-cells (lower left quadrant) may be decreased in the magnetically enriched cells, but little would be done to increase the ratio of single versus double positively staining B-cells. An alternative approach would therefore be to magnetically label the membrane vesicles of the non- or mock-transfected cell line and deplete these from the stained sample before FACS sorting. This could possibly be combined with light chain positive B-cell depletion in a single step.

A magnetic labelling strategy for vesicles could be performed similarly to the one used for fluorescently labelling them: that is, to label the cell line(s) internally (i.e. cytoplasmic) before vesicles are prepared. Internal magnetic labelling of cells has been performed by allowing phagocytotic cells to internalize magnetic microparticles, or when using a non-phagocytotic cell type, using particle guns and standard DNA transfection reagents. As probably virtually none of the cell lines used for B-cell selection as described here will be phagocytotic, the use of the former technique would be precluded. The latter two techniques mentioned have demonstrated moderate success rates, result in low signal intensities and require cumbersome devices or costly reagents. An alternative, non-limiting loading technique, which can also be applied in the context of the present invention (e.g. in a manner which will be clear to the skilled person based on the disclosure herein, and which may for example be analogous to the methods described in these cited references) and which could be more convenient in practice, is described a series of publications by Lee Josephson and Ralph Weissleder (notably J. Immunol. Meth., 2001, 256: 89-105). Briefly, this method consists of immobilizing cell penetrating peptides on magnetic nanoparticles and simply co-incubating the cells to be labeled with the nanoparticles. Cell penetrating peptides form a class of unrelated peptides, isolated from very different origins, where all of them can migrate through the cell membrane of live cells. The mechanism by which this occurs is still poorly described, but does not seem to require phagocytosis, nor does it seem to require energy by the cell. Importantly, virtually all cell types tested can be targeted by these peptides, and even “cargo” much larger than the peptides themselves can be co-transported into the cell when attached to the peptides. In the publication quoted above, the authors describe linking a well-known HIV Tat protein derived cell penetrating peptide to magnetic particles used as an MRI contrast agent. Co-incubation of these particles with normal human T-cells resulted in internalization of up to 30,000 beads per cell, with virtually all cells in the population staining to a homogeneous intensity. Importantly, internalization did not result in diminished viability or loss of function both in vitro and in vivo. Electron microscopy clearly demonstrated intracellular (predominantly nuclear) staining in small bead clusters. Other cell penetrating peptides show different trafficking patterns, indicating immobilization of an alternative peptide (such as a oligo-arginine homopolymer) to the magnetic particles may result in more cytoplasmic localization. Quasi-covalent immobilization of peptides to suitably small magnetic particles, such as Miltenyi Biotech MACS beads, can be performed using the biotin/streptavidin system; streptavidin-precoated beads are readily available. Biotinylation of a synthetically produced cell penetrating peptide such as poly-Arg can be incorporated into the peptide synthesis procedure itself, yielding essentially pure, homogenously and site-specifically biotinylated peptide which can be conjugated to the beads by simple co-incubation in solution. Directly linking a penetrating peptide to the bead surface without a long linker has already been shown to render the transport peptide far less efficient. Therefore, incorporating a long linker between the peptide and the biotin moiety during the synthesis step allows the peptide to extend from the bead surface, leaving the function of the peptide fully intact. Coated beads can then be quality assessed once and stored for future use in cell labellings.

Thus, immobilization of cell penetrating peptides on magnetic particles yields a reagent that can be used for easy in vitro magnetic labelling of live cells in the context of the present invention (e.g. in an analogous manner which will be clear to the skilled person based on the disclosure herein). Vesiculation from such magnetically labeled cells, especially when performed before, after or even concurrent to fluorescent labelling as described above, will yield bimodal fluorescent/magnetic detection/isolation reagents. Such bimodal reagents will be of great interest to either deplete membrane-bound antigen specific B-cells from unwanted specificities, or enrich the relevant fraction from the total pool of cells in the sample prior to FACS sorting.

Other FACS techniques that are suitable in the context of the present invention (e.g. in an analogous manner which will be clear to the skilled person based on the disclosure herein) are for example described in J. Immunol. Meth. 1989, 117: 275 or are known in the art (such as B-D's FACS 440), and include the presently available high-speed sorters (such as Dako-Cytomation's MoFlo; B-D's FACSaria; or Beckman-Coulter's Altras; reference is also made to J. Immunol. Meth., 2000, 243: 13); also, Daugherty et al. (J. Immunol. Meth., 2000, 243: 211) give a review of cell display library selection using flow cytometry sorters, which techniques can also be used in the context of the present invention (e.g. in an analogous manner which will be clear to the skilled person based on the disclosure herein).

In addition, or alternatively, techniques known per se other than high throughput flow cytometry can be used in the context of the present invention to enrich PBMC samples for antigen binding B-cells. Immobilizing the antigen on magnetic microbeads (such as those from Miltenyi Biotech and Dynal Biotech, to name only two major suppliers) or on another solid phase such as standard disposable tissue culture plasticware allows “panning” of tens of millions of cells against the antigen simultaneously, using generally available equipment well-known in the art. Subsequently washing away non-binding cells from the plastic, or holding the cells plus beads suspension to a magnet and pipetting off all non-bound cells, separates the vast majority of irrelevant non-B-cells (such as T-cells, granulocytes, NK cells, etc.) and non-binding B-cells from the few cells of interest. The procedure takes only a few hours in total, with most of the time required actually consisting of the incubation steps. The publication series from H. Leyendeckers et al. (for instance, Eur. J. Immunol., 2002, 32: 3126 and Eur. J. Immunol., 1999, 29: 1406) describe such methods for obtaining cell populations of high purity, as well as near-perfect preservation of viability and in vitro B-cell stimulation after panning.

Also, one or more of the above techniques can be suitably combined. For example, solid phase B-cell panning can be combined with the use flow cytometers or vice versa. The publication from N. N. Gangopadhyay et al. (J. Immunol. Meth., 2004, 292: 73) vividly illustrates the power of combining two such methodologies to get highly specific isolation (using flow cytometry sorting) of very rare cells (pre-enriched before FACS using cell panning techniques).

Again, all techniques described above or any suitable combination thereof can be used in the context of the present invention (e.g. in an analogous manner which will be clear to the skilled person based on the disclosure herein).

There are a number of publications on reagents directed against markers on human leucocyte subsets but found to be cross-reactive to llama and other camelids (Vet. Immunol. Immunopathol. 2000, 74: 17; J. Camel Prac. Res., 1998, 5: 179; J. Comp. Path., 2000, 127, 69; US 2002/0155604 A1), which could be applied in the context of the present invention (e.g. in a manner which will be clear to the skilled person based on the disclosure herein, and which may for example be analogous to the methods described in these cited references). It should be noted that the latter publication also describes a very simple method for discriminating conventional versus non-conventional B-cells, using commercially available reagents.

William Davis (Vet. Immunol. Immunopathol., 2000, 74, 103) has described the development of a set of monoclonal antibodies raised against llama leucocytes. This panel of monoclonals unambiguously identifies all major llama leucocyte subsets and can be obtained commercially, which could be applied in the context of the present invention (e.g. in a manner which will be clear to the skilled person based on the disclosure herein, and which may for example be analogous to the methods described in these cited references).

Also, several suppliers (such as Bethyl Labs, Montgomery, Tex., Triple J Farms division of Kent Labs, kentlabs.com/triplejproducts.html) provide ready-to-use labeled goat anti-llama immunoglobulin polyclonals, which can be used in the context of the present invention to identify surface immunoglobulins present on llama B-cells (e.g. in a manner which will be clear to the skilled person based on the disclosure herein, and which may for example be analogous to the methods described in these cited references).

Also, embedding all cells in a given sample in a gelatinous matrix droplet (live and individually) makes it possible to capture the soluble products produced by only a few cells around them (such as immunoglobulins from rare B-cells) and detect these using a fluorescence microscope or flow cytometer (see One Cell Systems, onecell.com/GMDProteinSecretionAssay.htm for schemata and details). This method and apparatus can also be applied in the context of the present invention (e.g. in a manner which will be clear to the skilled person based on the disclosure herein, and which may for example be analogous to the methods described in these cited references). Also, this particular non-limiting aspect does not require the of any cell population specific markers, obviating the need for llama specific reagents entirely.

Furthermore, an “affinity capture matrix” can be created on the surface of immunoglobulin producing B-cells using a methodology similar to the cytokine secreting detection methods described by R. Manz and colleagues (PNAS, 1995, 92: 1921; see also Miltenyi Biotech, miltenyibiotec.com/macs/principle/2 for schemata and details) or S. Carroll (J. Immunol. Methods, 2005, 296: 171). The first method requires the use of a bifunctional reagent, binding both a B-cell surface marker (not required to be unique to B-cells) and immunoglobulin. Such reagents can readily be created by combining those commercially available as mentioned in the second and third points set out above. These methods can again be applied in the context of the present invention (e.g. in a manner which will be clear to the skilled person based on the disclosure herein, and which may for example be analogous to the methods described in these cited references), in which the method described by Carroll et al. could be particularly convenient.

Thus, a wide variety of suitable reagents has been described in the prior art, and can be applied in the context of the present invention (e.g. in a manner which will be clear to the skilled person based on the disclosure herein, and which may for example be analogous to the methods described in these cited references). Many of these reagents are readily available from commercial sources.

As mentioned above, according to one non-limiting aspect of the invention, the B-cells are not cultured (expanded/propagated) before the immunoglobulin genes can be isolated and characterized. For example, the immunoglobulin sequences can be obtained directly from the single cells using any suitable technique known per se, such as a suitable amplification techniques, for example single cell PCR. Single cell PCR on immunoglobulins has been described well over a decade ago (see for example Nature, 1991, 350: 502; and see J. Immunol. Meth., 2000, 243: 25 for review of cytometry/single cell PCR combinations specifically).

It has also been described how such single cell PCR can be performed in a high-throughput fashion, using a flow cytometer and the product in question (RNAture GenePlates, mature.com/pdf/an118.pdf). In this respect, it should be noted that this reference describes single cell PCR for classical four-chain B-cells, necessitating cloning two genes from every cell. By contrast, in the present invention, only a single gene fragment needs to be cloned per cell to obtain the sequence of interest, thus reducing the level of technical difficulty of single cell cloning procedure.

Thus, in general, single cell PCR techniques, reagents, protocols and equipment known per se can be used in the context of the present invention (e.g. in a manner which will be clear to the skilled person based on the disclosure herein, and which may for example be analogous to the methods described in these cited references).

An non-limiting alternative to single cell PCR comprises the cultivation and stimulation of B-cells, for example through the use of B-cell stimulation feeder cell lines are commonly referred to in the literature. EL4-B5 is a mouse thymoma cell line, generated by Dr. Zubler of Geneva, Switzerland (Eur. J. Immunol., 1987, 17: 887). This cell line has been described to stimulate not only mouse B-cells, but also human B-cells (Eur. J. Immunol., 1987, 17: 887; J. Immunol. Methods, 1993, 160: 117). According to one non-limiting aspect of the present invention, llama B-cells can be stimulated using this cell line, and in doing so, it has been found that llama cells can be stimulated as efficiently as mouse B-cells (e.g. in a manner which will be clear to the skilled person based on the disclosure herein, and which may for example be analogous to the methods described in these cited references).

An alternative system is described by J. Banchereau (Dardilly, France) and relied initially on immobilizing agonistic anti-CD40 antibodies on the surface of Fc gamma receptor transfected fibroblasts (Science, 1991, 251: 70). Later variants of this technique employed CD40L transfected cells, once this gene was cloned and sequenced (J. Exp. Med., 1992, 176: 1543). In the research that has led to the present invention, the llama CD40L sequence was obtained and cloned this into a mammalian expression vector. Two camel cell lines (DUBCA, publicly available through ATCC code CRL-2276 and CAKI) have been transfected and llama CD40L overexpressing clones have been selected.

As another non-limiting alternative, recombinant proteins encoding extracellular fragments of mouse and human CD40L have been prepared in E. coli and used to stimulate B-cells without the need for a feeder cell line (J. Biol. Chem. 1995, 270: 7025). In the research leading up to the present invention, the homologous llama CD40L fragment sequence has been cloned into an E. coli expression plasmid as a source of recombinant CD40L protein for use in B-cell detection by FACS as well as B-cell stimulation in culture without requiring the use of feeder cells.

Llama cytokines, required for B-cell stimulation with or without feeder cells, can readily be obtained from stimulated llama PBMC samples. The supernatant of stimulated human PBMC also proved to be quite effective in our hands. Also, the llama cytokine gene sequences described by Odbileg et al. (Vet Immunol Immunopathol 2004, 102: 93; Vet Immunol Immunopathol 2005, 104: 145) allow for a very cost-effective mass-production of these cytokines as recombinant proteins in E. coli or any other host cell, and these cytokines can also be used to stimulate the antigen-producing B-cells described herein.

Thus, according to the invention, the murine EL4-B5 cell line could be used to stimulate species other than mouse, for example llama B-cells. Alternatively, publicly available llama cytokine gene sequences can be used, together with a publicly available camel-derived cell line.

As also further described hereinbelow, the V_{HH} domains identified using the method of the invention can be used as a Nanobody. Alternatively, the amino acid sequence and/or nucleotide sequence of a heavy chain antibody or a V_{HH} domain identified using the methods described above can be used as a starting point for generating Nanobodies; and/or a nucleic acid encoding such a heavy chain antibody or V_{HH} domain can be used as a starting material for generating such a Nanobody.

Thus, for example, in the above methods, after heavy chain antibodies against the desired antigen have been generated (in or after any step described as part of the methods above), the amino acid sequence of such a heavy chain antibody or of an antigen-binding fragment thereof (such as a V_{HH} domain) can be determined using a suitable sequencing technique, upon which a Nanobody with said amino acid sequence or with an amino acid sequence based thereon can be prepared using one of the methods described above. Similarly, nucleic acids encoding such a heavy chain antibody or V_{HH} domain generated above can be sequenced, after which said sequence can be used as a starting point for preparing (a nucleic acid encoding) a Nanobody.

The invention therefore also relates to such a Nanobody, to nucleic acids/nucleotide sequences encoding the same, to proteins or polypeptides comprising one or more of such Nanobodies and to nucleic acids/nucleotide sequences encoding such proteins or polypeptides; as well as to uses of such Nanobodies, polypeptides and proteins and methods for the preparation thereof.

In this respect, it will be clear to the skilled person that, although the nucleic acids/nucleotide sequences obtained by the above methods, as well as the V_{HH} domains and multivalent or multispecific obtained by the expression thereof, form preferred aspects of the invention, once the nucleotide sequence or amino acid sequence thereof, respectively, has been determined, there are various other ways of obtaining such V_{HH} domains or Nanobodies based thereon, to obtain proteins or polypeptides comprising such V_{HH} domains or Nanobodies, as well as nucleotide sequences/nucleic acids encoding the same.

Thus, the invention in its broadest sense is not limited to a specific way of generating the V_{HH} domains, Nanobodies, proteins, polypeptides, nucleic acids or genetic constructs described herein, and some preferred, but non-limiting methods are described hereinbelow.

According to another aspect, the invention relates to a nucleic acid that has been identified, selected, generated and/or cloned using one of the above methods. Said nucleic acid is preferably in the form of a genetic construct, as defined below. Such a genetic construct, comprising at least one such a nucleic acid and optionally one or more further elements of genetic constructs known per se, also forms an aspect of the invention.

The invention also relates to a method for producing a heavy chain antibody or antigen-binding fragment thereof, said method comprising expressing a nucleic acid as described above or a genetic construct as described above in a suitable host cell or host organism.

The invention also relates to a host cell or host organism, comprising such a nucleic acid and/or genetic construct. Preferably, said host cell or host organism expresses or produces, or under suitable conditions is capable of expressing or producing, the desired heavy chain antibody or antigen-binding fragment thereof.

The invention also relates to heavy chain antibody or an antigen-binding fragment thereof, encoded by a nucleic acid as described above or by a genetic construct as described above, obtained by the above method. Said heavy chain antibody or antigen-binding fragment thereof is preferably a V_{HH} domain.

The invention also relates to a Nanobody, the amino acid sequence of which is based on the amino acid sequence of the above V_{HH} domain and/or is based on amino acid sequence encoded by the above nucleic acid.

The invention also relates to a protein or polypeptide, containing or comprising at least one V_{HH} domain as described above and/or at least one Nanobody as described above; as well as to a nucleic acid or nucleotide sequence, encoding such a protein or polypeptide. Said nucleic acid is preferably in the form of a genetic construct, as defined below.

The invention also relates to a method for producing a protein or polypeptide containing or comprising at least one V_{HH} domain and/or at least one Nanobody, said method comprising expressing the above nucleic acid or the above genetic construct in a suitable host cell or host organism; as well as to a host cell or host organism, comprising the above nucleic acid according or the above genetic construct. Preferably, said host cell or host organism expresses or produces, or under suitable conditions is capable of expressing or producing, the desired protein or polypeptide.

Further aspects of the invention will become clear from the detailed description below.

### DETAILED DESCRIPTION OF THE INVENTION

The above and other aspects and embodiments of the invention will become clear from the further description hereinbelow, in which:

a) Unless indicated or defined otherwise, all terms used have their usual meaning in the art, which will be clear to the skilled person. Reference is for example made to the standard handbooks, such as Sambrook et al, “Molecular Cloning: A Laboratory Manual” (2nd. Ed.), Vols. 1-3, Cold Spring Harbor Laboratory Press (1989); F. Ausubel et al, eds., “Current protocols in molecular biology”, Green Publishing and Wiley Interscience, New York (1987); Roitt et al., “Immunology” (6th. Ed.), Mosby/Elsevier, Edinburgh (2001); and Janeway et al., “Immunobiology” (6^{th} Ed.), Garland Science Publishing/Churchill Livingstone, New York (2005), as well as to the general background art cited above.

b) Unless indicated otherwise, the term “immunoglobulin sequence”—whether it used herein to refer to a heavy chain antibody or to a conventional 4-chain antibody—is used as a general term to include both the full-size antibody, the individual chains thereof, as well as all parts, domains or fragments thereon (including but not limited to antigen-binding domains or fragments such as V_{HH} domains or V_{H}/V_{L} domains, respectively). In addition, the term “sequence” as used herein (for example in terms like “immunoglobulin sequence”, “antibody sequence”, “variable domain sequence”, “V_{HH} sequence” or “protein sequence”), should generally be understood to include both the relevant amino acid sequence as well as nucleic acid sequences or nucleotide sequences encoding the same, unless the context requires a more limited interpretation.

c) Unless indicated otherwise, all methods, steps, techniques and manipulations that are not specifically described in detail can and have been performed in a manner known per se, as will be clear to the skilled person. Reference is for example again made to the standard handbooks and the general background art referred to above and to the further references cited therein;

d) Amino acid residues will be indicated according to the standard three-letter or one-letter amino acid code, as mentioned in Table 1.

TABLE 1
one-letter and three-letter amino acid code

| Nonpolar, | Alanine | Ala | A |
|---|---|---|---|
| uncharged | Valine | Val | V |
| (at pH 6.0-7.0)⁽³⁾ | Leucine | Leu | L |
| | Isoleucine | Ile | I |
| | Phenylalanine | Phe | F |
| | Methionine⁽¹⁾ | Met | M |
| | Tryptophan | Trp | W |
| | Proline | Pro | P |
| Polar, | Glycine⁽²⁾ | Gly | G |
| uncharged | Serine | Ser | S |
| (at pH 6.0-7.0) | Threonine | Thr | T |
| | Cysteine | Cys | C |
| | Asparagine | Asn | N |
| | Glutamine | Gln | Q |
| | Tyrosine | Tyr | Y |
| Polar, | Lysine | Lys | K |
| charged | Arginine | Arg | R |
| (at pH 6.0-7.0) | Histidine⁽⁴⁾ | His | H |
| | Aspartate | Asp | D |
| | Glutamate | Glu | E |
| Notes: | | | |
| ⁽¹⁾Sometimes also considered to be a polar uncharged amino acid. | | | |
| ⁽²⁾Sometimes also considered to be a nonpolar uncharged amino acid. | | | |
| ⁽³⁾As will be clear to the skilled person, the fact that an amino acid residue is referred to in this Table as being either charged or uncharged at pH 6.0 to 7.0 does not say anything | | | |
| # about the charge said amino acid residue may have at a pH lower than 6.0 and/or higher than 7.0; the amino acid residues mentioned in the Table can be either charged and/or uncharged at | | | |
| # such a higher or lower pH, as will be clear to the skilled person. | | | |

⁽⁴⁾As is known in the art, the charge of a His residue is greatly dependant upon even small shifts in pH, but can generally be considered essentially uncharged at a pH of about 6.5.

e) For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position).

Alternatively, the degree of sequence identity between two or more nucleotide sequences may be calculated using a known computer algorithm for sequence alignment such as NCBI Blast v2.0, using standard settings.

Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A.

Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence;

f) For the purposes of comparing two or more amino acid sequences, the percentage of “sequence identity” between a first amino acid sequence and a second amino acid sequence may be calculated by dividing [the number of amino acid residues in the first amino acid sequence that are identical to the amino acid residues at the corresponding positions in the second amino acid sequence] by [the total number of amino acids in the first amino acid sequence] and multiplying by [100%], in which each deletion, insertion, substitution or addition of an amino acid residue in the second amino acid sequence—compared to the first amino acid sequence—is considered as a difference at a single amino acid residue (position).

Alternatively, the degree of sequence identity between two amino acid sequences may be calculated using a known computer algorithm, such as those mentioned above for determining the degree of sequence identity for nucleotide sequences, again using standard settings.

Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-2 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein.

Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Natl. Acad. Sci. USA 81: 140-144, 1984; Kyte & Doolittle; J Molec. Biol. 157: 105-132, 198 1, and Goldman et al., Ann. Rev. Biophys. Chem. 15: 321-353, 1986, all incorporated herein in their entirety by reference.

Information on the primary, secondary and tertiary structure of Nanobodies is given in the description below and in the general background art cited above. Also, for this purpose, the crystal structure of a V_{HH} domain from a llama is for example given by Desmyter et al., Nature Structural Biology, Vol. 3, 9, 803 (1996); Spinelli et al., Natural Structural Biology (1996); 3, 752-757; and Decanniere et al., Structure, Vol. 7, 4, 361 (1999).

g) Amino acid sequences and nucleic acid sequences are said to be “exactly the same” if they have 100% sequence identity (as defined above) over their entire length.

h) A nucleic acid sequence or amino acid sequence is considered to be “(in) essentially isolated form)”—for example, compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid sequence or amino acid sequence is considered “essentially isolated” when it has been purified at least 2-fold, in particular at least 10-fold, more in particular at least 100-fold, and up to 1000-fold or more. A nucleic acid sequence or amino acid sequence that is “in essentially isolated form” is preferably essentially homogeneous, as determined using a suitable technique, such as a suitable chromatographic technique, such as polyacrylamide-gel electrophoresis.

i) An amino acid sequence—such as a Nanobody, antibody or generally an antigen binding protein, polypeptide or fragment thereof—that can bind to, that has affinity for and/or that has specificity for a specific antigen or protein, or for at least one part, fragment or epitope thereof, is said to be “directed against” said antigen or protein;

j) As further described hereinbelow, the amino acid sequence and structure of a Nanobody can be considered—without however being limited thereto—to be comprised of four framework regions or “FR's”, which are referred to in the art and hereinbelow as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively.

k) As also further describe hereinbelow, the total number of amino acid residues in a Nanobody can be in the region of 110-120, is preferably 112-115, and is most preferably 113. It should however be noted that parts, fragments or analogs (as further described hereinbelow) of a Nanobody are not particularly limited as to their length and/or size, as long as such parts, fragments or analogs meet the further requirements outlined hereinbelow and are also preferably suitable for the purposes described herein.

l) The amino acid residues of a Nanobody are numbered according to the general numbering for V_{H} domains given by Kabat et al. (“Sequence of proteins of immunological interest”, US Public Health Services, NIH Bethesda, Md., Publication No. 91), as applied to V_{HH} domains from Camelids in Riechmann and Muyldermans, referred to above (see for example FIG. 2 of said reference). According to this numbering, FR1 of a Nanobody comprises the amino acid residues at positions 1-30, CDR1 of a Nanobody comprises the amino acid residues at positions 31-36, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-113. [In this respect, it should be noted that—as is well known in the art for V_{H} domains and for V_{HH} domains—the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position 1 according to the Kabat numbering corresponds to the start of FR1 and visa versa, position 36 according to the Kabat numbering corresponds to the start of FR2 and visa versa, position 66 according to the Kabat numbering corresponds to the start of FR3 and visa versa, and position 103 according to the Kabat numbering corresponds to the start of FR4 and visa versa.].

Alternative methods for numbering the amino acid residues of V_{H} domains, which methods can also be applied in an analogous manner to V_{HH} domains from Camelids and to Nanobodies, are the method described by Chothia et al. (Nature 342, 877-883 (1989)), the so-called “AbM definition” and the so-called “contact definition”. However, in the present description, claims and figures, the numbering according to Kabat as applied to V_{HH} domains by Riechmann and Muyldermans will be followed, unless indicated otherwise.

m) The Figures, Sequence Listing and the Experimental Part/Examples are only given to further illustrate the invention and should not be interpreted or construed as limiting the scope of the invention and/or of the appended claims in any way, unless explicitly indicated otherwise herein.

As mentioned above, the methods of the invention can be used to obtain V_{HH} domains that can be used as Nanobodies, or that can be used as a starting point for obtaining Nanobodies.

In this respect, it should however be noted that, as will become clear from the further description below, the term “Nanobody” as used herein encompasses more than the naturally occurring V_{HH} domains from species of Camelids, and for example also comprises non-naturally occurring analogs of naturally occurring V_{HH} domains (such as the “humanized” Nanobodies already referred to above and further described below); as well as parts or fragments of such naturally occurring V_{HH} domains or such non-naturally occurring analogs, again as further described below.

According to a specific embodiment, the term “Nanobody” as used herein does not encompass polypeptides with an amino acid sequence that is exactly the same as the amino acid sequence of a naturally occurring V_{H} domain, such as the amino acid sequence of a naturally occurring V_{H} domain from a mammal, and in particular from a human being.

As is also known from the references mentioned above, and as will be further described below, heavy chain antibodies and their V_{HH} domains can for example be obtained from species of Camelids, such as a camel, dromedary, llama and the other species of Camelids mentioned hereinbelow. It should however also be noted that the invention in its broadest sense is not limited to any specific source for obtaining the Nanobodies used herein, nor to any specific method for obtaining the Nanobodies used herein or to any method for obtaining polypeptides of the invention. For example, according to one preferred, but-non-limiting embodiment further described below, the polypeptides of the invention are obtained by means of a synthetic or semi-synthetic nucleic acid of the invention in a suitable bacterial expression system. Some other preferred, but non-limiting methods for obtaining the Nanobodies used herein and/or for obtaining the polypeptides of the invention will be described hereinbelow.

One particularly preferred class of such analogs are naturally occurring V_{HH} domains which have been “humanized”, as described in more detail below, i.e. by replacing one or more amino acid residues in the amino acid sequence of said V_{HH} domain by one or more of the amino acid residues that occur at the corresponding position in a V_{H} domain from a conventional 4-chain antibody from a human (e.g. indicated above). This can be performed in a manner known per se, which will be clear to the skilled person, for example on the basis of the further description below.

It should also be noted that Nanobodies can for example also be obtained by “camelizing” a naturally occurring V_{H} domain from another species of mammal (i.e. a V_{H} domain from a naturally occurring conventional 4-chain antibody) such as from a human being, i.e. by replacing one or more amino acid residues in the amino acid sequence of said V_{H} domain by one or more of the amino acid residues indicated above, so as to provide a Nanobody as defined hereinabove. This can be performed in a manner known per se, which will be clear to the skilled person, for example as described in WO 94/04678 or as further described below. Such camelization may preferentially occur at amino acid positions which are present at the V_{H}-V_{L} interface and at the so-called Camelidae hallmark residues (see for example also WO 94/04678), as also mentioned below.

For example, again as further described below, both “humanization” and “camelization” can be performed by providing a nucleotide sequence that encodes such a naturally occurring V_{HH} domain or V_{H} domain, respectively, and then changing, in a manner known per se, one or more codons in said nucleotide sequence such that the new nucleotide sequence encodes the humanized or camelized Nanobody, respectively, and then expressing the nucleotide sequence thus obtained in a manner known per se so as to provide the desired Nanobody. Alternatively, based on the amino acid sequence of a naturally occurring V_{HH} domain or V_{H} domain, respectively, the amino acid sequence of the desired humanized or camelized Nanobody, respectively, can be designed and then synthesized de novo using techniques for peptide synthesis known per se. Moreover, the amino acid sequence of a naturally occurring V_{HH} domain can be identified using the methods of the invention as disclosed herein, the camelizing substitutions in the V_{HH}/Nanobodies so obtained can be determined, and then V_{H} domains that contain these substitutions can be designed and synthesized in a manner known per se. Also, based on the amino acid sequence or nucleotide sequence of a naturally occurring V_{HH} domain or V_{H} domain, respectively, a nucleotide sequence encoding the desired humanized or camelized Nanobody, respectively, can be designed and then synthesized de novo using techniques for nucleic acid synthesis known per se, after which the nucleotide sequence thus obtained can be expressed in a manner known per se so as to provide the desired Nanobody.

Other suitable ways and techniques for obtaining Nanobodies and/or nucleotide sequences and/or nucleic acids encoding the same, starting from (the amino acid sequence of) naturally occurring V_{H} domains or preferably V_{HH} domains and/or from nucleotide sequences and/or nucleic acid sequences encoding the same will be clear from the skilled person, and may for example comprising combining one or more amino acid sequences and/or nucleotide sequences from naturally occurring V_{H} domains (such as one or more FR's and/or CDR's) with one or more one or more amino acid sequences and/or nucleotide sequences from naturally occurring V_{HH} domains (such an one or more FR's or CDR's), in a suitable manner so as to provide (a nucleotide sequence or nucleic acid encoding) a Nanobody.

The term Nanobodies as used herein in its broadest sense also comprises parts or fragments of the Nanobodies (including analogs) as defined above, which can again be as further described below. Generally, such parts or fragments of the Nanobodies and/or analogs will have amino acid sequences in which, compared to the amino acid sequence of the corresponding full length Nanobody or analog, one or more of the amino acid residues at the N-terminal end, one or more amino acid residues at the C-terminal end, one or more contiguous internal amino acid residues, or any combination thereof, have been deleted and/or removed. It is also possible to combine one or more of such parts or fragments to provide a Nanobody of the invention. Preferably, the amino acid sequence of a Nanobody that comprises one or more parts or fragments of a full length Nanobody and/or analog should have a degree of sequence identity of at least 50%, preferably at least 60%, more preferably at least 70%, such as at least 80%, at least 90% or at least 95%, with the amino acid sequence of the corresponding full length Nanobody. Also, the amino acid sequence of a Nanobody that comprises one or more parts or fragments of a full length Nanobody and/or analog is preferably such that is comprises at least 10 contiguous amino acid residues, preferably at least 20 contiguous amino acid residues, more preferably at least 30 contiguous amino acid residues, such as at least 40 contiguous amino acid residues, of the amino acid sequence of the corresponding full length Nanobody.

According to one particularly preferred embodiment, such a part or fragment comprises at least FR3, CDR3 and FR4 of the corresponding full length Nanobody, i.e. as for example described in the International application WO 03/050531 (Lasters et al.).

The amino acid sequences of CDR1, CDR2 and CDR3 of the Nanobodies used herein can be any CDR sequences that provide the Nanobodies with affinity, and preferably with specificity, against a desired antigen or a fragment or epitope thereof. Such amino acid sequences may for example be derived from (the amino acid sequence of) any suitable variable domain from any animal (in particular mammal), for example from a suitable heavy chain variable domain or from a suitable light chain variable domain from a mammal, and in particular from a suitable heavy chain variable domain, from an antibody raised against the desired antigen. For example, the CDR's may correspond to the amino acid sequences from a naturally occurring human V_{H} domain from an antibody raised against the desired antigen. It may also be possible to use artificial and/or synthetically derived CDR sequences, for example selected/obtained in a manner known per se.

According to one preferred embodiment, one or more, and preferably all, of the amino acid sequences of CDR1, CDR2 and CDR3 of the Nanobodies used herein correspond to the amino acid sequences of CDR1, CDR2 and CDR3, respectively, of the V_{HH} domain of a heavy chain antibody directed against the desired antigen or a fragment or epitope thereof. These amino acid sequences can generally be obtained by immunizing a Camelid with the desired antigen or a fragment or epitope thereof (in which said part or fragment is preferably such that it is capable of eliciting an immune response in said Camelid) in a manner so as to elicit an immune response against said antigen(s), and then determining the amino acid sequence of the respective CDR loops of the V_{HH} domains of the heavy chain antibodies directed against said antigen(s) in a manner known per se. In this context, without limitation and as further described below, the methods described herein will allow the cloning of the nucleotide sequences encoding said V_{HH} domains.

It is also possible, although usually less preferred, to combine in a Nanobody CDR's from different sources, one or more CDR's from one or more naturally occurring V_{HH} domains, and/or one or more CDR's from one or more naturally occurring V_{H} domains (such as human V_{H} domains), and/or one or more synthetic CDR's. Methods and techniques for obtaining (nucleotide sequences and/or nucleic acids encoding) the one or more CDR's and methods and techniques for combining said CDR's with (the nucleotide sequences and/or nucleic acids encoding) suitable FR's so as to provide a Nanobody will be clear to the skilled person, and include the methods described herein.

In accordance with the above, and according to one preferred, but non-limiting aspect of the aspect of the invention, a Nanobody in its broadest sense can be defined as a polypeptide comprising:
a) an amino acid sequence that is comprised of four framework regions/sequences interrupted by three complementarity determining regions/sequences, in which the amino acid residue at position 108 according to the Kabat numbering is Q;
   and/or:
b) an amino acid sequence that is comprised of four framework regions/sequences interrupted by three complementarity determining regions/sequences, in which the amino acid residue at position 44 according to the Kabat numbering is E and/or in which the amino acid residue at position 45 according to the Kabat numbering is an and/or:
c) an amino acid sequence that is comprised of four framework regions/sequences interrupted by three complementarity determining regions/sequences, in which the amino acid residue at position 103 according to the Kabat numbering is chosen from the group consisting of P, R and S, and is in particular chosen from the group consisting of R and S.

In particular, according to one preferred, but non-limiting aspect of the aspect of the invention, a Nanobody can be defined as a polypeptide comprising an amino acid sequence that is comprised of four framework regions/sequences interrupted by three complementarity determining regions/sequences, in which;
a-1) the amino acid residue at position 44 according to the Kabat numbering is chosen from the group consisting of G, E, D, G, Q, R, S, L; and is preferably chosen from the group consisting of G, E or Q; and
a-2) the amino acid residue at position 45 according to the Kabat numbering is chosen from the group consisting of L, R or C; and is preferably chosen from the group consisting of L or R; and
a-3) the amino acid residue at position 103 according to the Kabat numbering is chosen from the group consisting of W, R or S; and is preferably W or R, and is most preferably W;
a-4) the amino acid residue at position 108 according to the Kabat numbering is Q;
   or in which:
b-1) the amino acid residue at position 44 according to the Kabat numbering is chosen from the group consisting of E and Q; and
b-2) the amino acid residue at position 45 according to the Kabat numbering is R; and
b-3) the amino acid residue at position 103 according to the Kabat numbering is chosen from the group consisting of W, R and S; and is preferably W;
b-4) the amino acid residue at position 108 according to the Kabat numbering is chosen from the group consisting of Q and L; and is preferably Q;
   or in which:
c-1) the amino acid residue at position 44 according to the Kabat numbering is chosen from the group consisting of G, E, D, Q, R, S and L; and is preferably chosen from the group consisting of G, E and Q; and
c-2) the amino acid residue at position 45 according to the Kabat numbering is chosen from the group consisting of L, R and C; and is preferably chosen from the group consisting of L and R; and
c-3) the amino acid residue at position 103 according to the Kabat numbering is chosen from the group consisting of P, R and S; and is in particular chosen from the group consisting of R and S; and
c-4) the amino acid residue at position 108 according to the Kabat numbering is chosen from the group consisting of Q and L; is preferably Q.

Two particularly preferred, but non-limiting groups of the abovementioned Nanobodies are those according to a) above; according to a-1) to a-4) above; according to b) above; according to b-1) to b-4) above; according to c) above; and/or according to c-1) to c-4) above, in which;
a) the amino acid residues at positions 44-47 according to the Kabat numbering form the sequence GLEW (SEQ ID NO:8) and the amino acid residue at position 108 is Q;
   or in which:
b) the amino acid residues at positions 43-46 according to the Kabat numbering form the sequence KERE (SEQ ID NO:9) or KQRE (SEQ. ID NO:10) and the amino acid residue at position 108 is Q or L, and is preferably Q.

In the Nanobodies in which the amino acid residues at positions 43-46 according to the Kabat numbering form the sequence KERE (SEQ ID NO:9) or KQRE (SEQ ID NO:10), the amino acid residue at position 37 is most preferably F. In the Nanobodies in which the amino acid residues at positions 44-47 according to the Kabat numbering form the sequence GLEW (SEQ ID NO:8), the amino acid residue at position 37 is chosen from the group consisting of Y, H, I, V or F, and is most preferably V.

Thus, without being limited hereto in any way, on the basis of the amino acid residues present on the positions mentioned above, the Nanobodies used herein can generally be classified is on the basis of the following three groups:
a) The “GLEW-group”: Nanobodies with the amino acid sequence GLEW (SEQ ID NO:8) at positions 44-47 according to the Kabat numbering and Q at position 108 according to the Kabat numbering. As further described herein, Nanobodies within this group usually have a V at position 37, and can have a W, P, R or S at position 103, and preferably have a W at position 103. The GLEW group also comprises some GLEW-like sequences such as those mentioned in Table 2 below;
b) The “KERE-group”: Nanobodies with the amino acid sequence KERE (SEQ ID NO:9) or KQRE (SEQ ID NO:10) or at positions 43-46 according to the Kabat numbering and Q or L at position 108 according to the Kabat numbering. As further described herein, Nanobodies within this group usually have a F at position 37, an L or F at position 47; and can have a W, P, R or S at position 103, and preferably have a W at position 103;
c) The “103 P, R, S-group”: Nanobodies with a P, R or S at position 103. These Nanobodies can have either the amino acid sequence GLEW (SEQ ID NO:8) at positions 44-47 of the Kabat numbering or the amino acid sequence KERE (SEQ ID NO:9) or KQRE (SEQ ID NO:10) at positions 43-46 according to the Kabat numbering, the latter most preferably in combination with an F at position 37 and an L or an F at position 47 (as defined for the KERE-group); and can have Q or L at position 108 according to the Kabat numbering, and preferably have Q.

Also, more generally and in addition to the 108Q, 43E/44R and 103P,R,S residues mentioned above, Nanobodies can contain, at one or more positions that, in a conventional V_{H} domain, would form (part of) the V_{H}/V_{L} interface, contain-one or more amino acid residues that are more highly charged than the amino acid residues that naturally occur at the same position(s) in the corresponding naturally occurring V_{H} or V_{HH} domain, and in particular one or more charged amino acid residues (as mentioned in Table 1).

Such substitutions include, but are not limited to the GLEW-like sequences mentioned in Table 2 below; as well as the substitutions that are described in the International Application WO 00/29004 for so-called “microbodies”, e.g. a Q at position 108 and KLEW (SEQ ID NO:11) at positions 44-47.

In certain preferred Nanobodies, the amino acid residue at position 11 is chosen from the group consisting of L, M, S, V and W; and is preferably L.

Also, in certain preferred Nanobodies, the amino acid residue at position 83 is chosen from the group consisting of R, K, N, E, I and Q; and is most preferably either K or E (for Nanobodies corresponding to naturally occurring V_{HH} domains) or R (for “humanized” Nanobodies, as described below). The amino acid residue at position 84 is chosen from the group consisting of P, A, R, S, D and V, and is most preferably P (for Nanobodies corresponding to naturally occurring V_{HH} domains) or R (for “humanized” Nanobodies, as described below).

Furthermore, in certain preferred Nanobodies, the amino acid residue at position 104 is chosen from the group consisting of G and D; and is most preferably G.

Collectively, the amino acid residues at positions 11, 37, 44, 45, 47, 83, 84, 103, 104 and 108, which in the Nanobodies are as mentioned above, will also be referred to herein as the “Hallmark Residues”. The Hallmark Residues and the amino acid residues at the corresponding positions of the most closely related human V_{H} domain, V_{H}3, are summarized in Table 2.

Some especially preferred combinations of these Hallmark Residues as occur in naturally occurring V_{HH} domains are mentioned in Table 3. For comparison, the corresponding amino acid residues of the human V_{H}3 called DP-47 have been indicated in italics.

TABLE 2
Hallmark Residues in Nanobodies

| Position | Human V_{H}3 | Hallmark Residues |
|---|---|---|
| 11 | L, V; predominantly | L, M, S, V, W; preferably L |
| | L | |
| 37 | V, I, F; usually V | F⁽¹⁾, Y, H, I, L or V, preferably F^{(1) or Y} |
| 44⁽⁸⁾ | G | G⁽²⁾, E⁽³⁾, A, D, Q, R, S, L; |
| | | preferably G⁽²⁾, E⁽³⁾ or Q; |
| | | most preferably G⁽²⁾ or E⁽³⁾. |
| 45⁽⁸⁾ | L | L⁽²⁾, R⁽³⁾, C, I, L, P, Q, V; preferably L⁽²⁾ |
| | | or R⁽³⁾ |
| 47⁽⁸⁾ | W, Y | W⁽²⁾, L⁽¹⁾ or F⁽¹⁾, A, G, I, M, R, S, V or |
| | | Y; preferably W⁽²⁾, L⁽¹⁾, F⁽¹⁾ or R |
| 83 | R or K; usually R | R, K⁽⁵⁾, N, E⁽⁵⁾, G, I, M, Q or T; |
| | | preferably K or R; most preferably K |
| 84 | A, T, D; | P⁽⁵⁾, A, L, R, S, T, D, V; preferably P |
| | predominantly A | |
| 103 | W | W⁽⁴⁾, P⁽⁶⁾, R⁽⁶⁾, S; preferably W |
| 104 | G | G or D; preferably G |
| 108 | L, M or T; | Q, L⁽⁷⁾ or R; preferably Q or L⁽⁷⁾ |
| | predominantly L | |
| Notes: | | |
| ⁽¹⁾In particular, but not exclusively, in combination with KERE (SEQ ID NO: 9) or KQRE (SEQ ID NO: 10) at positions 43-46. | | |
| ⁽²⁾Usually as GLEW (SEQ ID NO: 8) at positions 44-47. | | |
| ⁽³⁾Usually as KERE (SEQ ID NO: 9) or KQRE (SEQ ID NO: 10) at positions 43-46, e.g. as KEREL (SEQ ID NO: 12), KEREF (SEQ ID NO: 13), KQREL (SEQ ID NO: 14), KQREF (SEQ ID NO: 15) or KEREG (SEQ ID NO: 16) at positions 43-47. Alternatively, also sequences such as TERE (SEQ ID NO: 17) (for example TEREL (SEQ ID NO: 18)), KECE | | |
| # (SEQ ID NO: 19) (for example KECEL (SEQ ID NO: 20) or KECER (SEQ ID NO: 21)), RERE (SEQ ID NO: 22) (for example REREG (SEQ ID NO: 23)), QERE (SEQ ID NO: 24) (for example QEREG (SEQ ID NO: 25)), KGRE (SEQ ID NO: 26) (for example KGREG | | |
| # (SEQ ID NO: 27)), KDRE (SEQ ID NO: 28) (for example KDREV (SEQ ID NO: 29)) are possible. Some other possible, but less preferred sequences include for example DECKL (SEQ ID NO: 30) and NVCEL (SEQ ID NO: 31). | | |
| ⁽⁴⁾With both GLEW (SEQ ID NO: 8) at positions 44-47 and KERE (SEQ ID NO: 9) or KQRE (SEQ ID NO: 10) at positions 43-46. | | |
| ⁽⁵⁾Often as KP or EP at positions 83-84 of naturally occurring V_{HH} domains. | | |
| ⁽⁶⁾In particular, but not exclusively, in combination with GLEW at positions | | |
| # 44-47. | | |
| ⁽⁷⁾With the proviso that when positions 44-47 are GLEW, position 108 is always Q. | | |
| ⁽⁸⁾The GLEW group also contains GLEW-like sequences at positions 44-47, such as for example GVEW (SEQ ID NO: 32), EPEW (SEQ ID NO: 33), GLER (SEQ ID NO: 34), DQEW (SEQ ID NO: 35), DLEW (SEQ ID NO: 36), GIEW (SEQ ID NO: 37), ELEW (SEQ | | |
| # ID NO: 38), GPEW (SEQ ID NO: 39), EWLP (SEQ ID NO: 40), GPER (SEQ ID NO: 41), and GLER (SEQ ID NO: 42). | | |

TABLE 3
Some preferred combinations of Hallmark Residues in naturally
occurring Nanobodies.
For humanization of these combinations, reference is made
to the specification.

| | 11 | 37 | 44 | 45 | 47 | 83 | 84 | 103 | 104 | 108 |
|---|---|---|---|---|---|---|---|---|---|---|
| DP-47 (human) | M | V | G | L | W | R | A | W | G | L |
| “KERE” group | L | F | E | R | L | K | P | W | G | Q |
| | L | F | E | R | F | E | P | W | G | Q |
| | L | F | E | R | F | K | P | W | G | Q |
| | L | Y | Q | R | L | K | P | W | G | Q |
| | L | F | L | R | V | K | P | Q | G | Q |
| | L | F | Q | R | L | K | P | W | G | Q |
| | L | F | E | R | F | K | P | W | G | Q |
| “GLEW” group | L | V | G | L | W | K | S | W | G | Q |
| | M | V | G | L | W | K | P | R | G | Q |
| “KERE” group | L | F | E | R | L | K | P | W | G | Q |
| | L | F | E | R | F | E | P | W | G | Q |
| | L | F | E | R | F | K | P | W | G | Q |
| | L | Y | Q | R | L | K | P | W | G | Q |
| | L | F | L | R | V | K | P | Q | G | Q |
| | L | F | Q | R | L | K | P | W | G | Q |
| | L | F | E | R | F | K | P | W | G | Q |
| “GLEW” group | L | V | G | L | W | K | S | W | G | Q |
| | M | V | G | L | W | K | P | R | G | Q |

In the Nanobodies, each amino acid residue at any other position than the Hallmark Residues can be any amino acid residue that naturally occurs at the corresponding position (according to the Kabat numbering) of a naturally occurring V_{HH} domain.

Such amino acid residues will be clear to the skilled person. Tables 4-7 mention some non-limiting residues that can be present at each position (according to the Kabat numbering) of the FR1, FR2, FR3 and FR4 of naturally occurring V_{HH} domains. For each position, the amino acid residue that most frequently occurs at each position of a naturally occurring V_{HH} domain (and which is the most preferred amino acid residue for said position in a Nanobody) is indicated in bold; and other preferred amino acid residues for each position have been underlined (note: the number of amino acid residues that are found at positions 26-30 of naturally occurring V_{HH} domains supports the hypothesis underlying the numbering Chothia (supra) that the residues at these positions already form part of CDR1.)

In Tables 4-7, some of the non-limiting residues that can be present at each position of a human V_{H}3 domain have also been mentioned. Again, for each position, the amino acid residue that most frequently occurs at each position of a naturally occurring human V_{H}3 domain is indicated in bold; and other preferred amino acid residues have been underlined.

TABLE 4
Non-limiting examples of amino acid residues in FR1

| | Amino acid residue(s): | | V_{HH} | V_{HH} |
|---|---|---|---|---|
| Pos. | Human V_{H}3 | Camelid V_{HH}'s | Ent. | Var. |
| 1 | E, Q | Q, A, E | — | — |
| 2 | V | V | 0.2 | 1 |
| 3 | Q | Q, K | 0.3 | 2 |
| 4 | L | L | 0.1 | 1 |
| 5 | V, L | Q, E, L, V | 0.8 | 3 |
| 6 | E | E, D, Q, A | 0.8 | 4 |
| 7 | S, T | S, F | 0.3 | 2 |
| 8 | G, R | G | 0.1 | 1 |
| 9 | G | G | 0 | 1 |
| 10 | G, V | G, D, R | 0.3 | 2 |
| 11 | Hallmark residue: L, M, S, V, W; preferably L | | 0.8 | 2 |
| 12 | V, I | V, A | 0.2 | 2 |
| 13 | Q, K, R | Q, E, K, P, R | 0.4 | 4 |
| 14 | P | A, Q, A, G, P, S, T, V | 1 | 5 |
| 15 | G | G | 0 | 1 |
| 16 | G, R | G, A, E, D | 0.4 | 3 |
| 17 | S | S, F | 0.5 | 2 |
| 18 | L | L, V | 0.1 | 1 |
| 19 | R, K | R, K, L, N, S, T | 0.6 | 4 |
| 20 | L | L, F, I, V | 0.5 | 4 |
| 21 | S | S, A, F, T | 0.2 | 3 |
| 22 | C | C | 0 | 1 |
| 23 | A, T | A, D, E, P, S, T, V | 1.3 | 5 |
| 24 | A | A, I, L, S, T, V | 1 | 6 |
| 25 | S | S, A, F, P, T | 0.5 | 5 |
| 26 | G | G, A, D, E, R, S, T, V | 0.7 | 7 |
| 27 | F | S, F, R, L, P, G, N, | 2.3 | 13 |
| 28 | T | N, T, E, D, S, I, R, A, G, R, F, Y | 1.7 | 11 |
| 29 | F, V | F, L, D, S, I, G, V, A | 1.9 | 11 |
| 30 | S, D, G | N, S, E, G, A, D, M, T | 1.8 | 11 |

For the footnotes, see the footnotes to Table 2.

TABLE 5
Non-limiting examples of amino acid residues in FR2

| | Amino acid residue(s): | | V_{HH} | V_{HH} |
|---|---|---|---|---|
| Pos. | Human V_{H}3 | Camelid V_{HH}'s | Ent. | Var. |
| 36 | W | W | 0.1 | 1 |
| 37 | Hallmark residue: F⁽¹⁾, H, I, L, Y or V, | | 1.1 | 6 |
| | preferably F⁽¹⁾ or Y | | | |
| 38 | R | R | 0.2 | 1 |
| 39 | Q | Q, H, P, R | 0.3 | 2 |
| 40 | A | A, F, G, L, P, T, V | 0.9 | 7 |
| 41 | P, S, T | P, A, L, S | 0.4 | 3 |
| 42 | G | G, E | 0.2 | 2 |
| 43 | K | K, D, E, N, Q, R, T, V | 0.7 | 6 |
| 44 | Hallmark residue: G⁽²⁾, E⁽³⁾, A, | | 1.3 | 5 |
| | D, Q, R, S, L; preferably G⁽²⁾, E⁽³⁾ | | | |
| | or Q; most preferably G⁽²⁾ or E⁽³⁾. | | | |
| 45 | Hallmark residue: L⁽²⁾, R⁽³⁾, C, | | 0.6 | 4 |
| | I, L, P, Q, V; preferably L⁽²⁾ or R⁽³⁾ | | | |
| 46 | E, V | E, D, K, Q, V | 0.4 | 2 |
| 47 | Hallmark residue: W⁽²⁾, L⁽¹⁾ or F⁽¹⁾, A, | | 1.9 | 9 |
| | G, I, M, R, S, V or Y; preferably | | | |
| | W⁽²⁾, L⁽¹⁾, F⁽¹⁾ or R | | | |
| 48 | V | V, I, L | 0.4 | 3 |
| 49 | S, A, G | A, S, G, T, V | 0.8 | 3 |

For the footnotes, see the footnotes to Table 2.

TABLE 6
Non-limiting examples of amino acid residues in FR3

| | Amino acid residue(s): | | V_{HH} | V_{HH} |
|---|---|---|---|---|
| Pos. | Human V_{H}3 | Camelid V_{HH}'s | Ent. | Var. |
| 66 | R | R | 0.1 | 1 |
| 67 | F | F, L, V | 0.1 | 1 |
| 68 | T | T, A, N, S | 0.5 | 4 |
| 69 | I | I, L, M, V | 0.4 | 4 |
| 70 | S | S, A, F, T | 0.3 | 4 |
| 71 | R | R, G, H, I, L, K, Q, S, T, W | 1.2 | 8 |
| 72 | D, E | D, E, G, N, V | 0.5 | 4 |
| 73 | N, D, G | N, A, D, F, I, K, L, R, S, T, V, Y | 1.2 | 9 |
| 74 | A, S | A, D, G, N, P, S, T, V | 1 | 7 |
| 75 | K | K, A, E, K, L, N, Q, R | 0.9 | 6 |
| 76 | N, S | N, D, K, R, S, T, Y | 0.9 | 6 |
| 77 | S, T, I | T, A, E, I, M, P, S | 0.8 | 5 |
| 78 | L, A | V, L, A, F, G, I, M | 1.2 | 5 |
| 79 | Y, H | Y, A, D, F, H, N, S, T | 1 | 7 |
| 80 | L | L, F, V | 0.1 | 1 |
| 81 | Q | Q, E, I, L, R, T | 0.6 | 5 |
| 82 | M | M, I, L, V | 0.2 | 2 |
| 82a | N, G | N, D, G, H, S, T | 0.8 | 4 |
| 82b | S | S, N, D, G, R, T | 1 | 6 |
| 82c | L | L, P, V | 0.1 | 2 |
| 83 | Hallmark residue: R, K⁽⁵⁾, N, E⁽⁵⁾, G, I, | | 0.9 | 7 |
| | M, Q or T; preferably K or R; most preferably K | | | |
| 84 | Hallmark residue: P⁽⁵⁾, A, D, L, R, S, T, V; | | 0.7 | 6 |
| | preferably P | | | |
| 85 | E, G | E, D, G, Q | 0.5 | 3 |
| 86 | D | D | 0 | 1 |
| 87 | T, M | T, A, S | 0.2 | 3 |
| 88 | A | A, G, S | 0.3 | 2 |
| 89 | V, L | V, A, D, I, L, M, N, R, T | 1.4 | 6 |
| 90 | Y | Y, F | 0 | 1 |
| 91 | Y, H | Y, D, F, H, L, S, T, V | 0.6 | 4 |
| 92 | C | C | 0 | 1 |
| 93 | A, K, T | A, N, G, H, K, N, R, S, T, V, Y | 1.4 | 10 |
| 94 | K, R, T | A, V, C, F, G, I, K, L, R, S or T | 1.6 | 9 |

For the footnotes, see the footnotes to Table 2.

TABLE 7
Non-limiting examples of amino acid residues in FR4

| | Amino acid residue(s): | | | |
|---|---|---|---|---|
| | Human | | V_{HH} | V_{HH} |
| Pos. | V_{H}3 | Camelid V_{HH}'s | Ent. | Var. |
| 103 | Hallmark residue: W⁽⁴⁾, P⁽⁶⁾, | | 0.4 | 2 |
| | R⁽⁶⁾, S; preferably W | | | |
| 104 | Hallmark residue: G or D; | | 0.1 | 1 |
| | preferably G | | | |
| 105 | Q, R | Q, E, K, P, R | 0.6 | 4 |
| 106 | G | G | 0.1 | 1 |
| 107 | T | T, A, I | 0.3 | 2 |
| 108 | Hallmark residue: Q, L⁽⁷⁾ or R; | | 0.4 | 3 |
| | preferably Q or L⁽⁷⁾ | | | |
| 109 | V | V | 0.1 | 1 |
| 110 | T | T, I, A | 0.2 | 1 |
| 111 | V | V, A, I | 0.3 | 2 |
| 112 | S | S, F | 0.3 | 1 |
| 113 | S | S, A, L, P, T | 0.4 | 3 |

For the footnotes, see the footnotes to Table 2.

Thus, according to one preferred, but non-limiting embodiment of the invention, a Nanobody as present in a Polypeptide of the Invention has the following structure:
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively,
   and in which

a) in which FR1 comprises the amino acid sequence:

| | |
|---|---|
| [1] QVQLQESGGGXVQAGGSLRLSCAASG [26] | (SEQ ID NO:43) |

b) FR2 comprises the amino acid sequence:

| | |
|---|---|
| [36] WXRQAPGKXXEXVA [49] | (SEQ ID NO:44) |

c) FR3 comprises the amino acid sequence:

| | |
|---|---|
| [66] RFTISRDNAKNTVYLQMNSLXXEDTAVYYCA | (SEQ ID NO:45) |
| A [94] | |

and

d) FR4 comprises the amino acid sequence:

| | |
|---|---|
| [103] XXQGTXVTVSS [113] | (SEQ ID NO:46) |

in which the Hallmark Residues are indicated by “X” and are as defined hereinabove and in which the numbers between brackets refer to the amino acid positions according to the Kabat numbering.

Nanobodies and nucleic acids encoding the same can be obtained in one of the manners indicated below for obtaining the polypeptides of the invention.

The term Nanobodies as used herein in its broadest sense also comprises natural or synthetic mutants, variants, alleles, analogs and orthologs (hereinbelow collectively referred to as “analogs”) of the Nanobodies as defined above.

In such analogs, the Hallmark Residues should be as defined above.

Generally, such analogs can for example comprise homologous sequences, functional portions, or a functional portion of an homologous sequence (as further defined below) of a Nanobody.

Also, generally, such analogs should be such that their framework regions have, in total, more than 80%, preferably more than 85%, more preferably more than 90%, even more preferably more than 95%, such as more than 96%, more than 97%, more than 98% or more than 99% sequence identity (as defined above) at the amino acid level (in which the Hallmark residues are not taken into account) with the amino acid sequences of the framework regions mentioned hereinabove.

Generally, in such analogs, each amino acid residue (other than the Hallmark Residue) in each of the framework regions can be replaced by any other amino acid residue, provided that the total degree of sequence identity of the framework regions remains as defined above. Preferably, however, in such analogs:
one or amino acid residues in the above framework sequences are replaced by one or more amino acid residues that naturally occur at the same position in a naturally occurring V_{HH} domain. Some examples of such substitutions are mentioned in Tables 4-7 above;
   and/or:
one or amino acid residues in the above framework sequences are replaced by one or more amino acid residues that can be considered a “conservative” amino acid substitution, as described hereinabove;
   and/or:
one or amino acid residues in the above framework sequences are replaced by one or more amino acid residues that naturally occur at the same position in a naturally occurring V_{H} domain of a human being. This is generally referred to as “humanization” of the naturally occurring V_{HH}/Nanobody in general and of said position in particular, and will be discussed in more detail hereinbelow;
   and:
positions for which only one amino acid residue is mentioned for both the V_{H} domain and the V_{HH} domain in Tables 4-7 above are preferably not replaced.

Also, although generally less preferred, in such analogs, one or more amino acid residues may be deleted from the framework regions and/or inserted into the framework regions (optionally in addition to one or more amino acid substitutions as mentioned above), provided that the total degree of sequence identity of the framework regions remains as defined above. The Hallmark residues should not be deleted. Also, most preferably, amino acid residues for which only one amino acid residue is mentioned for both the V_{H} domain and the V_{HH} domain in Tables 4-7 above are preferably not deleted.

Generally, analogs can for example be obtained by providing a nucleic acid that encodes a naturally occurring V_{HH} domain, changing the codons for the one or more amino acid residues that are to be humanized into the codons for the corresponding human amino acid residue(s), expressing the nucleic acid/nucleotide sequence thus obtained in a suitable host or expression system; and optionally isolating and/or purifying the analog thus obtained to provide said analog in essentially isolated form (as defined hereinabove). This can generally be performed using methods and techniques known per se, which will be clear to the skilled person, for example from the handbooks and references cited herein and/or from the further description hereinbelow. Alternatively, and for example, a nucleic acid encoding an analog can be synthesized in a manner known per se (for example using an automated apparatus for synthesizing nucleic acid sequences with a predefined amino acid sequence) and can be expressed in a suitable host or expression system, upon which the analog thus obtained can optionally be isolated and/or purified so as to provide said analog in essentially isolated form (as defined hereinabove). Another way to provide the analogs involves chemical synthesis of the pertinent amino acid sequence using techniques for peptide synthesis known per se, such as those mentioned hereinbelow.

It will be also generally be clear to the skilled person that Nanobodies (including analogs thereof) can also be prepared starting from human V_{H} sequences (i.e. amino acid sequences or the corresponding nucleotide sequences), such as for example human V_{H}3 sequences such as DP-47, DP-51 or DP-29, by changing one or more amino acid residues in the amino acid sequence of said human V_{H} domain, so as to provide an amino acid sequence that has (a) a Q at position 108; and/or (b) E at position 44 and/or R at position 45, and preferably E at position 44 and R at position 45; and/or (c) P, R or S at position 103, as described above. Again, this can generally be performed using the various methods and techniques referred to in the previous paragraph, using an amino acid sequence and/or nucleotide sequence for a human V_{H} domain as a starting point.

The term Nanobody as used herein in its broadest sense also encompasses parts or fragments of the full sequence-Nanobodies or full sequence-analogs described hereinabove. Preferably, such parts or fragments should be such that they still can bind to, have affinity for and/or have specificity for the antigen to which the corresponding Nanobody or analog with the full amino acid sequence (as described above) is directed, i.e. with an affinity and/or a specificity which is at least 10%, preferably at least 50%, more preferably at least 70%, even more preferably at least 80%, such as at least 90%, at least 95%, at least 99% or more, of the affinity and/or specificity of the corresponding Nanobody or analog with the full amino acid sequence, as determined using a suitable assay, for example an assay to determine binding of the part or fragment to the antigen, as will be clear to the skilled person.

Generally, parts or fragments of the Nanobodies and/or analogs have amino acid sequences in which, compared to the amino acid sequence of the corresponding full length Nanobody or analog, one or more of the amino acid residues at the N-terminal end, one or more amino acid residues at the C-terminal end, one or more contiguous internal amino acid residues, or any combination thereof, have been deleted and/or removed. It is also possible to combine one or more of such parts or fragments to provide a Nanobody of the invention.

Preferably, the amino acid sequence of a Nanobody that comprises one or more parts or fragments of a full length Nanobody and/or analog should have a degree of sequence identity of at least 50%, preferably at least 60%, more preferably at least 70%, such as at least 80%, at least 90% or at least 95%, with the amino acid sequence of the corresponding full length Nanobody.

Also, the amino acid sequence of a Nanobody that comprises one or more parts or fragments of a full length Nanobody and/or analog is preferably such that it comprises at least contiguous amino acid residues, preferably at least 20 contiguous amino acid residues, more preferably at least 30 contiguous amino acid residues, such as at least 40 contiguous amino acid residues, of the amino acid sequence of the corresponding full length Nanobody.

According to one particularly preferred embodiment, such a part or fragment comprises at least FR3, CDR3 and FR4 of the corresponding full length Nanobody, i.e. as for example described in the International application WO 03/050531 (Lasters et al.).

The amino acid sequences of CDR1, CDR2 and CDR3 of the Nanobodies can be any CDR sequences that provide the Nanobodies with affinity, and preferably with specificity, against the desired antigen or a fragment or epitope thereof.

From the description hereinabove, it will be clear that the amino acid sequences of the Nanobodies used herein differ at at least one amino acid position in at least one of the framework regions from the amino acid sequences of naturally occurring V_{H} domains, such as the amino acid sequences of naturally occurring V_{H} domains from antibodies from Camelids and/or human beings. In particular, it will be clear that the amino acid sequences of the Nanobodies used herein differ at at least one of the Hallmark Residues from amino acid sequences of naturally occurring V_{H} domains, such as the amino acid sequences of naturally occurring V_{H} domains from antibodies from Camelids and/or human beings.

From the description hereinabove, it will be clear that the amino acid sequences of the some of the Nanobodies used herein differ at at least one amino acid position in at least one of the framework regions from the amino acid sequences of naturally occurring V_{HH} domains. In particular, it will be clear that the amino acid sequences of the some of the Nanobodies used herein differ at at least one of the Hallmark Residues from amino acid sequences of naturally occurring V_{HH} domains. As already mentioned above one particular example of this type of Nanobodies are Nanobodies which have been humanized compared to the naturally occurring V_{HH} sequence, in the manner described above. Thus, according to one specific, but non-limiting embodiment, the term Nanobody as used herein does not comprise polypeptides in which the amino acid sequences of FR1, FR2, FR3 and FR4 are all exactly the same as the amino acid sequences of FR1, FR2, FR3 and FR4 of a naturally occurring V_{H} domain, such as the naturally occurring amino acid sequences of naturally occurring V_{H} domain from a mammal, such as a human being.

As mentioned above, the invention also relates to proteins or polypeptides comprising at least one V_{HH} domain (i.e. as identified using the methods of the invention) or at least one Nanobody based thereon.

According to one non-limiting embodiment of the invention, such a polypeptide of the invention essentially consists of a Nanobody. By “essentially consist of” is meant that the amino acid sequence of the polypeptide of the invention either is exactly the same as the amino acid sequence of a Nanobody (as mentioned above) or corresponds to the amino acid sequence of a Nanobody in which a limited number of amino acid residues, such as 1-10 amino acid residues and preferably 1-6 amino acid residues, such as 1, 2, 3, 4, 5 or 6 amino acid residues, have been added to the amino terminal end, to the carboxy terminal end, or both to the amino terminal end and to the carboxy terminal end of the amino acid sequence of the Nanobody.

Said amino acid residues may or may not change, alter or otherwise influence the (biological) properties of the Nanobody and may or may not add further functionality to the Nanobody. For example, said amino acid residues may:
a) form a “tag”, i.e. an amino acid sequence or residue that allows or facilitates the purification of the Nanobody, for example using affinity techniques directed against said sequence or residue. Thereafter, said sequence or residue may be removed (e.g. by chemical or enzymatical cleavage) to provide the nucleotide sequence of the invention (for this purpose, the sequence or residue may optionally be linked to the amino acid sequence of the invention via a cleavable linker sequence). Some preferred, but non-limiting examples of such residues are multiple histidine residues and glutathione residues,
b) may be one or more amino acid residues that can be provided with functional groups and/or that have been functionalized, in a manner known per se. For example, as is known in the art, amino acid residues such as lysine and in particular cysteine allow for the attachment of PEG groups (i.e., pegylate), which may mask surface sites on a protein and thus for example decrease immunogenicity, improve half-life in plasma and stabilize against proteolytic cleavage;
c) can be a N-terminal Met residue, for example as result of expression in a heterologous host cell or host organism.

According to another embodiment, a polypeptide of the invention can comprise the amino acid sequence of a Nanobody, which is fused at its amino terminal end, at its carboxy terminal end, or both at its amino terminal end and at its carboxy terminal end with at least one further amino acid sequence.

Again, said further amino acid sequence(s) may or may not change, alter or otherwise influence the (biological) properties of the Nanobody and may or may not add further functionality to the Nanobody.

For example, according to one preferred, but non-limiting embodiment, said further amino acid sequence may comprise at least one further Nanobody, so as to provide a polypeptide of the invention that comprises at least two, such as three, four or five, Nanobodies, in which said Nanobodies may optionally be linked via one or more linker sequences (as defined below).

Polypeptides of the invention comprising two or more Nanobodies will also referred to herein as “multivalent” polypeptides. For example a “bivalent” polypeptide of the Invention comprises two Nanobodies, optionally linked via a linker sequence, whereas a “trivalent” polypeptide of the invention comprises three Nanobodies, optionally linked via two linker sequences; etc.

In a multivalent polypeptide of the invention, the two or more Nanobodies may be the same or different. For example, the two or more Nanobodies in a multivalent polypeptide of the invention:
may be directed against the same antigen, i.e. against the same parts or epitopes of said antigen or against two or more different parts or epitopes of said antigen; and/or:
may be directed against the different antigens;
   or a combination thereof.

Thus, a bivalent polypeptide of the invention for example:
may comprise two identical Nanobodies;
may comprise a first Nanobody directed against a first part or epitope of an antigen and a second Nanobody directed against the same part or epitope of said antigen or against another part or epitope of said antigen;
or may comprise a first Nanobody directed against a first antigen and a second

Nanobody directed against a second antigen different from said first antigen;

whereas a trivalent Polypeptide of the Invention for example:

may comprises three identical or different Nanobodies directed against the same or different parts or epitopes of the same antigen;
may comprise two identical or different Nanobodies directed against the same or different parts or epitopes on a first antigen and a third Nanobody directed against a second antigen different from said first antigen; or
may comprise a first Nanobody directed against a first antigen, a second Nanobody directed against a second antigen different from said first antigen, and a third Nanobody directed against a third antigen different from said first and second antigen.

Polypeptides of the invention that contain at least two Nanobodies, in which at least one Nanobody is directed against a first antigen and at least one Nanobody is directed against a second antigen different from the first antigen, will also be referred to as “multispecific” Nanobodies. Thus, a “bispecific” Nanobody is a Nanobody that comprises at least one Nanobody directed against a first antigen and at least one further Nanobody directed against a second antigen, whereas a “trispecific” Nanobody is a Nanobody that comprises at least one Nanobody directed against a first antigen, at least one further Nanobody directed against a second antigen, and at least one further Nanobody directed against a third antigen; etc.

Accordingly, in their simplest form, a bispecific polypeptide of the invention is a bivalent polypeptide of the invention (as defined above), comprising a first Nanobody directed against a first antigen and a second Nanobody directed against a second antigen, in which said first and second Nanobody may optionally be linked via a linker sequence (as defined below); whereas a trispecific polypeptide of the invention in its simplest form is a trivalent polypeptide of the invention (as defined above), comprising a first Nanobody directed against a first antigen, a second Nanobody directed against a second antigen and a third Nanobody directed against a third antigen, in which said first, second and third Nanobody may optionally be linked via one or more, and in particular one and more in particular two, linker sequences.

However, as will be clear from the description hereinabove, the invention is not limited thereto, in the sense that a multispecific polypeptide of the invention may comprise any number of Nanobodies directed against two or more different antigens.

For multivalent and multispecific polypeptides containing one or more V_{HH} domains and their preparation, reference is also made to Conrath et al., J. Biol. Chem., Vol. 276, 10. 7346-7350, as well as to EP 0 822 985.

Linkers for use in multivalent and multispecific polypeptides will be clear to the skilled person, and for example include gly-ser linkers, for example of the type (glyₓser_{y})_{z}, such as for example (gly₄ser)₃ (SEQ ID NO:47) or (gly₃ser₂)₃ (SEQ ID NO:48), as described in WO 99/42077, hinge like regions such as the hinge regions of naturally occurring heavy chain antibodies or similar sequences. For other suitable linkers, reference is also made to the general background art cited above.

Linkers can also provide some functionality for the multivalent or multispecific polypeptide. For example, linkers containing one or more charged amino acid residues (see Table 1 above) can provide improved hydrophilic properties, whereas linkers that form or contain small epitopes or tags can be used for the purposes of detection, identification and/or purification.

As also further described herein, a multispecific polypeptide of the invention directed against a desired antigen and against at least one serum protein, such as the serum proteins mentioned hereinbelow, and in particular against human serum albumin, may show increased half-life in serum, compared to the corresponding monovalent Nanobody.

As mentioned hereinabove, the methods described herein are particularly suited for generating such multivalent or multispecific polypeptides of the invention.

In a polypeptide of the invention, the at least one Nanobody may also be linked to a conventional V_{H} domain or to a natural or synthetic analog of a V_{H} domain, optionally via a linker sequence.

In a polypeptide of the invention, the at least one Nanobody may also be linked to a V_{L} domain or to a natural or synthetic analog of a V_{L} domain, optionally via a linker sequence, so as to provide a polypeptide of the invention that is in the form analogous to a conventional scFv fragment, but containing a Nanobody instead of a V_{H} domain.

In a polypeptide of the invention, the at least one Nanobody may also be linked to one or more of a CH1, CH2 and/or CH3 domain, optionally via a linker sequence. For instance, a Nanobody linked to a suitable CH1 domain could for example be used—together with suitable light chains—to generate antibody fragments/structures analogous to conventional Fab fragments or F(ab′)₂ fragments, but in which one or (in case of an F(ab′)₂ fragment) one or both of the conventional V_{H} domains have been replaced by a Nanobody. Such fragments may also be heterospecific or bispecific, i.e. directed against two or more antigens. A Nanobody linked to suitable CH2 and CH3 domains, for example derived from Camelids, could be used to form a monospecific or bispecific heavy chain antibody. Finally, a Nanobody linked to suitable CH1, CH2 and CH3 domains, for example derived from a human being, could be used—together with suitable light chains—to form an antibody that is analogous to a conventional 4-chain antibody, but in which one or both of the conventional V_{H} domains have been replaced by a Nanobody.

Also, in addition to the one or more Nanobodies, polypeptides of the invention can also contain functional groups, moieties or residues, for example therapeutically active substances, such as those mentioned below, and/or markers or labels, such as fluorescent markers, isotopes, etc., as further described hereinbelow.

As will be clear to the skilled person, one particularly useful method for preparing a Nanobody and/or a polypeptide of the invention generally comprises the steps of:
the expression, in a suitable host cell or host organism (also referred to herein as a “host of the invention”) or in another suitable expression system of a nucleic acid that encodes said Nanobody or polypeptide of the invention (also referred to herein as a “nucleic acid of the invention”), optionally followed by:
isolating and/or purifying the Nanobody or polypeptide of the invention thus obtained.

In particular, such a method may comprise the steps of:
cultivating and/or maintaining a host of the invention under conditions that are such that said host of the invention expresses and/or produces at least one Nanobody and/or polypeptide of the invention; optionally followed by:
isolating and/or purifying the Nanobody or polypeptide of the invention thus obtained.

A nucleic acid of the invention can be in the form of single or double stranded DNA or RNA, and is preferably in the form of double stranded DNA. For example, the nucleotide sequences of the invention may be genomic DNA, cDNA or synthetic DNA (such as DNA with a codon usage that has been specifically adapted for expression in the intended host cell or host organism).

According to one embodiment of the invention, the nucleic acid of the invention is in essentially isolated form, as defined hereinabove.

The nucleic acid of the invention may also be in the form of, be present in and/or be part of a vector, such as for example a plasmid, cosmid or YAC, which again may be in essentially isolated form.

The nucleic acids of the invention can be prepared or obtained in a manner known per se, based on the information on the amino acid sequences for the polypeptides of the invention given herein, and/or can be isolated from a suitable natural source. To provide analogs, nucleotide sequences encoding naturally occurring V_{HH} domains can for example be subjected to site-directed mutagenesis, so at to provide a nucleic acid of the invention encoding said analog. Also, as will be clear to the skilled person, to prepare a nucleic acid of the invention, also several nucleotide sequences, such as at least one nucleotide sequence encoding a Nanobody and for example nucleic acids encoding one or more linkers can be linked together in a suitable manner.

Techniques for generating the nucleic acids of the invention will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers, using for example a sequence of a naturally occurring V_{H} or V_{HH} as a template. These and other techniques will be clear to the skilled person, and reference is again made to the standard handbooks, such as Sambrook et al. and Ausubel et al., mentioned above, as well as the Examples below.

The nucleic acid of the invention may also be in the form of, be present in and/or be part of a genetic construct, as will be clear to the person skilled in the art. Such genetic constructs generally comprise at least one nucleic acid of the invention that is optionally linked to one or more elements of genetic constructs known per se, such as for example one or more suitable regulatory elements (such as a suitable promoter(s), enhancer(s), terminator(s), etc.) and the further elements of genetic constructs referred to hereinbelow. Such genetic constructs comprising at least one nucleic acid of the invention will also be referred to herein as “genetic constructs of the invention”.

The genetic constructs of the invention may be DNA or RNA, and are preferably double-stranded DNA. The genetic constructs of the invention may also be in a form suitable for transformation of the intended host cell or host organism, in a form suitable for integration into the genomic DNA of the intended host cell or in a form suitable independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system).

In a preferred but non-limiting embodiment, a genetic construct of the invention comprises:

a) at least one nucleic acid of the invention; operably connected to:

b) one or more regulatory elements, such as a promoter and optionally a suitable terminator; and optionally also:

c) one or more further elements of genetic constructs known per se; in which the terms “regulatory element”, “promoter”, “terminator” and “operably connected” have their usual meaning in the art (as further described below); and in which said “further elements” present in the genetic constructs may for example be 3′- or 5′-UTR sequences, leader sequences, selection markers, expression markers/reporter genes, and/or elements that may facilitate or increase (the efficiency of) transformation or integration. These and other suitable elements for such genetic constructs will be clear to the skilled person, and may for instance depend upon the type of construct used, the intended host cell or host organism; the manner in which the nucleotide sequences of the invention of interest are to be expressed (e.g. via constitutive, transient or inducible expression); and/or the transformation technique to be used.

Preferably, in the genetic constructs of the invention, said at least one nucleic acid of the invention and said regulatory elements, and optionally said one or more further elements, are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promoter). Generally, when two nucleotide sequences are operably linked, they will be in the same orientation and usually also in the same reading frame. They will usually also be essentially contiguous, although this may also not be required.

Preferably, the regulatory and further elements of the genetic constructs of the invention are such that they are capable of providing their intended biological function in the intended host cell or host organism.

For instance, a promoter, enhancer or terminator should be “operable” in the intended host cell or host organism, by which is meant that (for example) said promoter should be capable of initiating or otherwise controlling/regulating the transcription and/or the expression of a nucleotide sequence—e.g. a coding sequence—to which it is operably linked (as defined above).

Some particularly preferred promoters include, but are not limited to, promoters known per se for the expression in bacterial cells, such as those mentioned hereinbelow and/or those used in the Examples.

A selection marker should be such that it allows—i.e. under appropriate selection conditions—host cells and/or host organisms that have been (successfully) transformed with the nucleotide sequence of the invention to be distinguished from host cells/organisms that have not been (successfully) transformed. Some preferred, but non-limiting examples of such markers are genes that provide resistance against antibiotics (such as kanamycin or ampicillin), genes that provide for temperature resistance, or genes that allow the host cell or host organism to be maintained in the absence of certain factors, compounds and/or (food) components in the medium that are essential for survival of the non-transformed cells or organisms.

A leader sequence should be such that—in the intended host cell or host organism—it allows for the desired post-translational modifications and/or such that it directs the transcribed mRNA to a desired part or organelle of a cell. A leader sequence may also allow for secretion of the expression product from said cell. As such, the leader sequence may be any pro-, pre-, or prepro-sequence operable in the host cell or host organism. Leader sequences may not be required for expression in a bacterial cell.

An expression marker or reporter gene should be such that—in the host cell or host organism—it allows for detection of the expression of (a gene or nucleotide sequence present on) the genetic construct. An expression marker may optionally also allow for the localisation of the expressed product, e.g. in a specific part or organelle of a cell and/or in (a) specific cell(s), tissue(s), organ(s) or part(s) of a multicellular organism. Such reporter genes may also be expressed as a protein fusion with the amino acid sequence of the invention. Some preferred, but non-limiting examples include fluorescent proteins such as GFP.

Some preferred, but non-limiting examples of suitable promoters, terminator and further elements include those used in the Examples below. For some (further) non-limiting examples of the promoters, selection markers, leader sequences, expression markers and further elements that may be present/used in the genetic constructs of the invention—such as terminators, transcriptional and/or translational enhancers and/or integration factors—reference is made to the general handbooks such as Sambrook et al. and Ausubel et al. mentioned above, as well as to the examples that are given in WO 95/07463, WO 96/23810, WO 95/07463, WO 95/21191, WO 97/11094, WO 97/42320, WO 98/06737, WO 98/21355, U.S. Pat. No. 6,207,410, U.S. Pat. No. 5,693,492 and EP 1 085 089. Other examples will be clear to the skilled person. Reference is also made to the general background art cited above and the further references cited hereinbelow.

The genetic constructs of the invention may generally be provided by suitably linking the nucleotide sequence(s) of the invention to the one or more further elements described above, for example using the techniques described in the general handbooks such as Sambrook et al. and Ausubel et al., mentioned above.

Often, the genetic constructs of the invention will be obtained by inserting a nucleotide sequence of the invention in a suitable (expression) vector known per se. Some preferred, but non-limiting examples of suitable expression vectors are those used in the Examples below, as well as for example:
vectors for expression in mammalian cells: pMAMneo (Clontech), pcDNA3 (Invitrogen), pMC1neo (Stratagene), pSG5 (Stratagene), EBO-pSV2-neo (ATCC 37593), pBPV-1 (8-2) (ATCC 37110), pdBPV-MMTneo (342-12) (ATCC 37224), pRSVgpt (ATCC37199), pRSVneo (ATCC37198), pSV2-dhfr (ATCC 37146), pUCTag (ATCC 37460) and 1ZD35 (ATCC-37565), as well as viral-based expression systems, such as those based on adenovirus;
vectors for expression in bacterial cells: pET vectors (Novagen) and pQE vectors (Qiagen);
vectors for expression in yeast or other fungal cells: pYES2 (Invitrogen) and Pichia expression vectors (Invitrogen);
vectors for expression in insect cells: pBlueBacII (Invitrogen) and other baculovirus vectors
vectors for expression in plants or plant cells: for example vectors based on cauliflower mosaic virus or tobacco mosaic virus, suitable strains of Agrobacterium, or Ti-plasmid based vectors;

The nucleic acids of the invention and/or the genetic constructs of the invention may be used to transform a host cell or host organism. The host or host cell may be any suitable (fungal, prokaryotic or eukaryotic) cell or cell line or any suitable fungal, prokaryotic or eukaryotic organism, for example:
a bacterial strain, including but not limited to strains of E. coli, Bacillus, Streptomyces and Pseudomonas;
a fungal cell, including but not limited to cells from species of Aspergillus, Trichoderma or other filamentous fungi;
a yeast cell, including but not limited to cells from species of Kluyveromyces or Saccharomyces;
an amphibian cell or cell line, such as Xenopus oocytes;
an insect-derived cell or cell line, such as cells/cell lines derived from lepidoptera, including but not limited to Spodoptera SF9 and Sf21 cells or cells/cell lines derived from Drosophila, such as Schneider and Kc cells; and/or
a plant or plant cell,
a mammalian cell or cell line, for example derived a cell or cell line derived from a human, from the mammals including but not limited to CHO- and BHK-cells and human cells or cell lines such as HeLa and COS;

Reference is also made to the general background art cited hereinabove, as well as to for example WO 94/29457; WO 96/34103; WO 99/42077; Riechmann, Journal of Immunological Methods 231 (1999), 25-39; van der Linden, Journal of Biotechnology 80 (2000), 261-270; Thomassen et al., Enzyme and Microbial Technology 30 (2002), 273-278.

As mentioned above, one of the advantages of the use of Nanobodies is that the polypeptides based thereon can be prepared through expression in a suitable bacterial system, and suitable bacterial expression systems, vectors, host cells, regulatory elements, etc., will be clear to the skilled person, for example from the references cited above. It should however be noted that the invention in its broadest sense is not limited to expression in bacterial systems.

Preferably, in the invention, an (in vivo or in vitro) expression system, such as a bacterial expression system, is used that provides the polypeptides of the invention in a form that is suitable for pharmaceutical use, and such expression systems will again be clear to the skilled person. As also will be clear to the skilled person, polypeptides of the invention suitable for pharmaceutical use can be prepared using techniques for peptide synthesis.

Suitable techniques for transforming a host of the invention will be clear to the skilled person and may depend on the intended host cell/host organism and the genetic construct to be used. Reference is again made to the handbooks and patent applications mentioned above.

After transformation, a step for detecting and selecting those host cells or host organisms that have been successfully transformed with the nucleotide sequence/genetic construct of the invention may be performed. This may for instance be a selection step based on a selectable marker present in the genetic construct of the invention or a step involving the detection of the amino acid sequence of the invention, e.g. using specific antibodies.

The transformed host cell (which may be in the form or a stable cell line) or host organisms (which may be in the form of a stable mutant line or strain) form further aspects of the present invention.

Preferably, these host cells or host organisms are such that they express, or are (at least) capable of expressing (e.g. under suitable conditions), an amino acid sequence of the invention (and in case of a host organism: in at least one cell, part, tissue or organ thereof). The invention also includes further generations, progeny and/or offspring of the host cell or host organism of the invention, that may for instance be obtained by cell division or by sexual or asexual reproduction.

To produce/obtain expression of the amino acid sequences of the invention, the transformed host cell or transformed host organism may generally be kept, maintained and/or cultured under conditions such that the (desired) amino acid sequence of the invention is expressed/produced. Suitable conditions will be clear to the skilled person and will usually depend upon the host cell/host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food and/or suitable nutrients, the use of a suitable temperature, and optionally the presence of a suitable inducing factor or compound (e.g. when the nucleotide sequences of the invention are under the control of an inducible promoter); all of which may be selected by the skilled person. Again, under such conditions, the amino acid sequences of the invention may be expressed in a constitutive manner, in a transient manner, or only when suitably induced.

It will also be clear to the skilled person that the amino acid sequence of the invention may (first) be generated in an immature form (as mentioned above), which may then be subjected to post-translational modification, depending on the host cell/host organism used. Also, the amino acid sequence of the invention may be glycosylated, again depending on the host cell/host organism used.

The amino acid sequence of the invention may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g. using a specific, cleavable amino acid sequence fused with the amino acid sequence of the invention) and/or preparative immunological techniques (i.e. using antibodies against the amino acid sequence to be isolated).

Generally, for pharmaceutical use, the polypeptides of the invention of the inventions may be formulated as a pharmaceutical preparation comprising at least one polypeptide of the invention and at least one pharmaceutically acceptable carrier, diluent or excipient and/or adjuvant, and optionally one or more further pharmaceutically active polypeptides and/or compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers for use in the preparation thereof, will be clear to the skilled person, and are further described hereinbelow.

It is also envisaged that the polypeptides of the invention and compositions containing the same may be of value in the veterinary field, which for the purposes herein not only includes the prevention and/or treatment of diseases in animals, but also—for economically important animals such as cattle, pigs, sheep, chicken, fish, etc.—enhancing the growth and/or weight of the animal and/or the amount and/or the quality of the meat or other products obtained from the animal. Thus, in a further aspect, the invention relates to a composition for veterinary use that contains at least one polypeptide of the invention and at least one suitable carrier (i.e. a carrier suitable for veterinary use), and optionally one or more further active substances.

### BRIEF DESCRIPTION OF THE FIGURES

FIG. 1: Detection of antigen-specific llama B-cells in selected and unselected populations.

FIG. 2: Recloning of individual Nanobody genes into expression vector pAX56b by site-specific recombination (Gateway@).

FIG. 3: Induced serum response against A431 tumor cells in immunized llamas.

FIGS. 4A and 4B: Serum response on human EGFR expressing mouse fibroblasts cells (Her14) versus the parental mouse fibroblasts NIH3T3 clone 2.2 acceptor cells (3T3) in immunized llamas 024/025 (panel A) and 026/027 (panel B).

FIGS. 5A and 5B: Serum response of immunized llamas 024/025 (panel A) and 026/027 (panel B) on purified human EGFR.

FIG. 6: Result of gel electrophoresis indicating reliable RNA isolation from small numbers of cells.

FIG. 7: Result of gel electrophoresis indicating reliable cDNA synthesis from small numbers of cells.

FIG. 8: Collagen selection in DBA mice: V kappa PCR on nucleic acid prepared from unselected and selected cells.

FIG. 9: HuClq selection: Amplification of Nanobody before enrichment and after selection.

FIG. 10: HuClq selection: Amplification of Nanobody long hinge to FR1 region, CH2 to FR1 region-before enrichment and after selection.

FIG. 11: HuCD28/Fc-gamma fusion protein selection: Leader-oligo-dT amplification selected fraction.

FIG. 12: HuCD28Fc-gamma fusion protein selection: FR1-hinge amplification selected fraction.

FIG. 13: HuCD28/Fc-gamma fusion protein selection: FR1-CH2 amplification selected fraction.

FIG. 14: Human integrin alpha-v-beta-5 selection: RNA integrity check.

FIG. 15: Human integrin alpha-v-beta-5 selection: cDNA quality control PCR.

FIG. 16: Cloning of Nanobody collection in the non-expression vector of pAX56a.

FIG. 17: Coomassie stained SDS-PAGE and Western blot results indicating the production of Nanobody in expression vector pAX56b.

FIG. 18: Binding assay results indicating the production of functional Nanobody in expression vector pAX56b.

FIG. 19A: Composition of the Nanobody sequence and position of the distal poly-A sequence.

FIG. 19B: PCR using FR1 and oligo-dT primer, restriction using BstEII, to provide Nanobody gene.

FIG. 20: Bivalent formatted Nanobodies

FIGS. 21A and 21B: Bispecific formatted Nanobodies: specificity A Nanobody N-terminal to specificity B Nanobody (FIG. 21A), and specificity B Nanobody N-terminal to specificity A Nanobody (FIG. 21B).

FIG. 22: Generation of fluorescent vesicles carrying a given membrane-bound protein, for use in flow cytometric B-cell detection and isolation.

FIGS. 23A and 23B: FIG. 23A reflects the B-cell repertoire possibly encountered in immune animal blood/lymph node etc. samples. FIG. 23B shows how staining a diverse repertoire of B-cells using fluorescent vesicles carrying a given membrane-bound protein gives rise to three distinct populations in bivariate FACS plots. Gating and subsequent sorting of the upper left quadrant contained cells selects only B-cells binding the transgenic protein of interest.

FIG. 24: Generation of magneto-fluorescent vesicles carrying membrane-bound protein repertoire of parental cell line, for use in pre-sorting magnetic depletion of irrelevant specificity B-cell depletion and detection during sorting. Poly-Arg is depicted as RRRR (SEQ ID NO:49) or RRRRR (SEQ ID NO:50).

FIGS. 25A and 25B: FIG. 25A shows a staining mix of B-cell specificities with magneto-fluorescent vesicles carrying membrane-bound protein repertoire of parental cell line, and non-magnetic fluorescent vesicles transgenic line. Magnet will retain only B-cells specific for irrelevant membrane-bound proteins present on transfection host cells. FIG. 25B shows bivariate plot B-cell populations after magnetically depleting irrelevant specificity B-cells before sorting. Patterns now resolve into only two populations: large population irrelevant T- and B-cells (lower left quadrant), and transgenic membrane protein specific B-cells (upper left quadrant).

FIGS. 26A-26D: FIG. 26A: A schematic comparison of production and secretion of soluble immunoglobulin by activated B-cells (“plasma cells”) and memory B-cells. FIG. 26B: Schematic of monoclonal antibody that specifically binds camelid non-conventional immunoglobulins in the presence of conventional immunogobulins (upper panel); this monoclonal antibody labeled with two different fluorescent dyes (lower panel). FIG. 26C: Schematic comparison of surface marker staining of PBMCs (upper panel) versus surface marker and intracellular staining of PBMCs (lower panel) to distinguish plasma cells and memory B-cells. FIG. 26D: Schematic diagram of protocol for isolating cells by surface marker and intracellular antibody-dye staining of PBMCs followed by surface marker and intracellular antigen-dye staining of PBMCs.

FIG. 27: Staining of heavy chain IgG displaying B-cells using fluorescent antigen (TNF), control staining without any Alexa488 stained reagent and control staining with irrelevant antigen, also Alexa 488 labeled.

FIG. 28: FACS analysis results of non-immune, immune and IL6 binding B-cell enriched immune animal PBL samples.

Experimental Part

Conventional antibodies are large multi-subunit protein molecules comprising at least four polypeptide chains. For example, human IgG has two heavy chains and two light chains that are disulfide bonded to form the functional antibody. The size of a conventional IgG is about 150 kD. Because of their relatively large size, complete antibodies (e.g., IgG, IgA, IgM, etc.) are limited in their therapeutic usefulness due to problems in, for example, tissue penetration. Considerable efforts have focused on identifying and producing smaller antibody fragments that retain antigen binding function and solubility.

The heavy and light polypeptide chains of antibodies comprise variable (V) regions or domains that directly participate in antigen interactions, and constant (C) regions or domains that provide structural support and function in non-antigen-specific interactions with immune effectors. Variable domains are also called antigen binding domains.

The antigen binding domain of a conventional antibody is comprised of two separate domains: a heavy chain variable domain (V_{H}) and a light chain variable domain (V_{L}: which can be either V kappa or V lambda). The antigen binding site itself is formed by six polypeptide loops, three from the V_{H} domain (H1, H2 and H3) and three from the V_{L} domain (L1, L2 and L3).

Within V_{H} and V_{L} there are hypervariable regions which show the most sequence variability from one antibody to another and framework regions which are less variable. Folding brings the hypervariable regions together to form the antigen-binding pockets. These sites of closest contact between antibody and antigen are the complementarity determining regions (CDR) of the antibody. It is believed that the third CDR region (CDR3) and especially the heavy chain CDR3 (CDR3) plays a key role in antibody specificity (Kabat and Wu, 1991) and is invariably involved in antigen-binding (WO 03/050531, incorporated hereby in its entirety by reference).

In vivo, a diverse primary repertoire of V genes that encode the V_{H} and V_{H} domains is produced by the combinatorial rearrangement of gene segments. C regions include the light chain C regions (referred to as CL regions) and the heavy chain C regions (referred to as CH1, CH2 and CH3 regions). While antibody V_{H} and V_{H} bind antigen, antibody constant regions determine its biological functions. CH2 domains bind complement and control the rate of Ig catabolism (breakdown). CH2 and CH3 domains bind phagocyte FcR (Fc Receptor) to stimulate antigen uptake. The biological functions of the C domains are independent of the antigen specificity of the molecule.

A number of smaller antigen binding fragments of naturally occurring antibodies have been identified following protease digestion. These include, for example, the “Fab fragment” (V_{L}-C_{L}+V_{H}-C_{H}1), “Fab′ fragment” (a Fab with the heavy chain hinge region) and “F(ab′)2 fragment” (a dimer of Fab′ fragments joined at the heavy chain hinge region). Such fragments can be made to eliminate the more immunogenic Fc region from the monoclonal antibodies; this also reduces their serum half life.

Recombinant methods have been used to generate even smaller antigen-binding fragments, referred to as “single chain Fv (variable fragment)” or “scFv,” consisting of V_{L} and V_{H} joined by a synthetic peptide linker. Toxin sequences replace the Fc region of antibody in engineered immunotoxins.

Other examples of genetic engineering to modify monoclonal antibodies include chimeric antibodies (mouse V_{H} and V_{L} gene segments of the desired specificity spliced to human C_{H} and C_{L} gene segments) and heteroconjugates (made with H-L pairs from different antibodies).

As the majority of antibody specificity and affinity resides in the CDR3 loop, small functional units of heavy chain variable regions have been described (e.g. WO 00/29004, U.S. Pat. No. 5,702,892). A unique class of heavy chain antibodies, which are naturally devoid of light chain sequences, can be derived from Camelids and are disclosed in WO 94/04678, the entirety of which is incorporated herein by reference.

The Camelidae heavy chain antibodies are found as homodimers of a single heavy chain, dimerized via their constant regions. The variable domains of these camelidae heavy chain antibodies are referred to as V_{HH} domains or V_{HH}, and can be either used per se as Nanobodies and/or as a starting point for obtaining Nanobodies. Isolated V_{HH}'s retain the ability to bind antigen with high specificity (Hamers-Casterman et al., 1993, Nature 363: 446-448). Such V_{HH} domains, or nucleotide sequences encoding them, can be derived from antibodies raised in Camelidae species, for example in camel, dromedary, llama, alpaca and guanaco. Other species besides Camelidae (e.g. shark, pufferfish) may produce functional antigen-binding heavy chain antibodies, from which (nucleotide sequences encoding) such naturally occurring V_{HH}'s can be obtained, e.g. using the methods described herein.

For use in therapy, human proteins are preferred, primarily because they are not as likely to provoke an immune response when administered to a patient. Isolated human V_{H} domains tend to be relatively insoluble and are often poorly expressed. Comparisons of camelid V_{HH} with the V_{H} domains of human antibodies reveals several key differences in the framework regions of the camelid V_{HH} domain corresponding to the V_{H}/V_{L} interface of the human V_{H} domains. Mutation of these human residues to V_{HH} resembling residues has been performed to produce “camelized” human V_{H} domains that retain antigen binding activity, yet have improved expression and solubility.

Libraries of single V_{H} domains have also been described, derived for example from murine V_{H} genes amplified from genomic DNA or from mRNA from the spleens of immunized mice and expressed in E. coli (Ward et al., 1989, Nature 341: 544-546). The isolated single V_{H} domains are called “dAbs” or domain antibodies. A “dAb” is an antibody single variable domain (V_{H} or V_{L}) polypeptide that specifically binds antigen. A “dAb” binds antigen independently of other V domains; however, as the term is used herein, a “dAb” can be present in a homo- or heteromultimer with other V_{H} or V_{L} domains where the other domains are not required for antigen binding by the dAb, i.e., where the dAb binds antigen independently of the additional V_{H} or V_{L} domains.

As used herein, an “antigen” is bound by an antibody or a binding region (e.g. a variable domain) of an antibody. Typically, antigens are capable of raising an antibody response in vivo. An antigen can be a peptide, polypeptide, protein, nucleic acid, lipid, carbohydrate, or other molecule, and includes multisubunit molecules. Generally, an immunoglobulin variable domain is selected for target specificity against a particular antigen.

Nanobodies

Camelidae express a unique, extensive repertoire of functional heavy chain antibodies that lack light chains. The V_{HH} molecules derived from Camelidae antibodies are the smallest intact antigen-binding domains known (approximately 15 kDa, or 10 times smaller than conventional IgG) and hence are well suited towards delivery to dense tissues and for accessing the limited space between macromolecules.

Nanobodies not only possess the advantageous characteristics of conventional antigen-binding proteins (such as full-size conventional 4-chain antibodies or the commonly used fragments and analogs thereof as described above, and full-size heavy chain antibodies), such as low toxicity and high selectivity, but they also exhibit additional favourable properties. They are soluble; as such they may be stored and/or administered in higher concentrations compared with conventional antigen-binding proteins, such as full-size conventional 4-chain antibodies or the commonly used fragments and analogs thereof. Nanobodies are stable at room temperature; as such they may be prepared, stored and/or transported without the use of refrigeration equipment, conveying a cost, time and environmental savings. Nanobodies resist harsh conditions, such as extreme pH, denaturing reagents and high temperatures, so making the Nanobodies suitable for delivery by oral administration. Nanobodies are resistant to the action of proteases, which is less the case for conventional antibodies. Nanobodies exhibit high binding affinity for a broad range of different antigen types and an ability to bind to epitopes not recognised by conventional antibodies, for example they display extended-CDR3 loops with the potential to penetrate into cavities (WO 97/49805). Nanobodies are ideal building blocks for the generation of bi- or multi-functional molecules by (head-to-tail) fusion, optionally via one or more liner sequences, as disclosed in WO 96/34103 (incorporated herein by reference). Through their small size, Nanobodies allow better tissue penetration and ability to reach all parts of the body than for example conventional antibodies. Specific Nanobodies from llamas have been shown in vitro to be able to cross the human blood-brain barrier (WO 02/057445).

Furthermore, Nanobodies are less immunogenic than conventional antigen-binding proteins, such as full-size conventional 4-chain antibodies obtained from non-human mammals or the commonly used fragments and analogs thereof. A subclass of Nanobodies from Camelidae has been discovered which displays up to 95% amino acid sequence homology to human V_{H} framework regions. This suggests that immunogenicity upon administration in human patients can be anticipated to be minor or even non-existent. Alternatively, if so required, humanization of Nanobodies requires only a few residues that need to be substituted.

As mentioned above, the method of the present invention may be used for the cloning and direct screening of immunoglobulin sequences (including but not limited to multivalent polypeptides comprising: two or more variable domains—or antigen binding domains—and in particular V_{H} domains or V_{HH} domains; fragments of V_{L}, V_{H} or V_{HH} domains, such as CDR regions, for example CDR3 regions; antigen-binding fragments of conventional 4-chain antibodies such as Fab fragments and scFv's, heavy chain antibodies and domain antibodies; and in particular of V_{H} sequences, and more in particular of V_{HH} sequences) that can be used as part of and/or to construct such multivalent constructs. According to one specific embodiment, the method of the invention can also be used to directly express and/or screen such multivalent constructs.

Multivalent Polypeptides Containing two or More Nanobodies

The scope of the invention not only encompasses isolated Nanobodies as described above, but also larger polypeptides that comprise one or more Nanobodies, or fragments thereof, as well as nucleotide sequences/nucleic acids encoding the same. These polyvalent/multivalent polypeptides can generally be as described in more detail in the International application PCT/BE2004/000159 for polyvalent polypeptides directed against EGFR.

The method of the present invention allows for very efficient direct screening of polyvalent (multivalent and/or multispecific) polypeptides, as is described further.

It has been demonstrated by the inventors (e.g. WO 04/041862, WO 04/062551) that multivalent polypeptides comprising at least two Nanobodies directed against multimeric targets or cell surface antigens have the advantage of unusually high functional affinity for the target, displaying several orders of magnitude higher than expected inhibitory properties compared to their monovalent counterparts. The functional affinities and antagonistic potency of these multivalent polypeptides are much higher than those reported in the prior art for conventional bivalent and multivalent antigen-binding proteins, such as ScFv fragments. Such polypeptides comprising two or more Nanobodies linked to each other directly or via a (short) linker sequence show the high functional affinities expected theoretically with multivalent conventional four-chain antibodies.

For TNF-alpha it was demonstrated that bivalent polypeptides comprising two Nanobodies directed against TNF-alpha were able to block two of the three receptor binding sites in the trimeric cytokines, thereby efficiently preventing the recruitment of two receptors necessary for signal transduction. The bivalent proteins form a complex with TNF without the formation of large aggregates which were found for monoclonal antibodies (like Remicade®), but instead such bivalent proteins give equivalent complexes as has been observed for the receptor itself (like for Enbrel®).

Modeling of the TNF-Nanobody structure suggests that due to their small size the Nanobody can penetrate into the receptor binding site. In addition two of these Nanobodies linked together via a short linker can bind simultaneously to a single trimer of a cytokine, which is impossible for a conventional 4-chain antibody because of sterical constraints.

Overall, these data explain the excellent antagonistic efficacy of bivalent TNF-alpha Nanobodies as measured in very sensitive bioassays.

Avidity is the functional affinity of multivalent antigen binding to multivalent antibody molecules. Avidity strengthens binding to antigens with repeating identical epitopes. The more antigen-binding sites an individual antibody molecule has, the higher its avidity for antigen.

Multivalent polypeptides comprising at least two Nanobodies have increased residence times and affinity/avidity towards their targets. These multivalent polypeptides can be altered to provide target-specific imaging agents or to improve half-life or to obtain effector functions by recruiting cells from the immune system responsible for clearance of the targeted antigens or cells or by inducing ADCC or CDC.

A multivalent polypeptide as described above refers to a polypeptide comprising two or more anti-target Nanobodies which have been covalently linked. The Nanobodies may be identical in sequence or may be different in sequence, but are directed against the same target or the same antigens or epitopes thereof. Alternatively, such multivalent constructs may be directed to different epitopes of the same target. Reference is again made to the International application PCT/BE2004/000159.

Depending on the number of Nanobodies linked, a multivalent polypeptide may be bivalent (two Nanobodies), trivalent (three Nanobodies), tetravalent (four Nanobodies) or may be of a higher valency number of molecules.

Thus, multivalent heavy chain antibodies provide a promising perspective for therapeutic and diagnostic drug design.

As further described hereinbelow, the method described herein can easily and advantageously be adapted for the cloning and direct screening of multivalent polypeptides comprising one or more immunoglobulin sequences or fragments thereof, including but not limited to multivalent polypeptides comprising: two or more variable domains (or antigen binding domains) and in particular V_{H} domains or V_{HH} domains; fragments of V_{L}, V_{H} or V_{HH} domains, such as CDR regions, for example CDR3 regions; antigen-binding fragments of conventional 4-chain antibodies such as Fab fragments and scFv's, heavy chain antibodies and domain antibodies (dAbs), and in particular of V_{H} sequences, and more in particular of V_{HH} sequences.

The method of the invention is preferably used for the cloning and direct screening of multivalent polypeptides comprising one or more V_{HH} domains or fragments thereof.

Multispecific Polypeptides Comprising two or More Nanobodies

The scope of the invention not only encompasses all of the above described isolated Nanobodies, or nucleotide sequences encoding the same, but also larger polypeptides that comprise one or more Nanobodies, or fragments thereof, fused to other polypeptides, such as serum protein(s).

Such bispecific polypeptides comprise two different binding specificities fused together and, in the most simple example, bind to two adjacent epitopes on a single target antigen, thereby increasing the avidity. Alternatively, such bispecific polypeptides can cross-link two different antigens and are powerful therapeutic reagents.

Multispecific (trispecific, tetraspecific, etc.) polypeptides comprise more than two (three, four, etc.) different binding specificities fused together and, in the most simple example, bind to two adjacent epitopes on a single target antigen, thereby increasing the avidity. Alternatively, such multispecific polypeptides can cross-link more than two different antigens and are powerful therapeutic reagents, particularly for recruitment of cytotoxic T cells for cancer treatment. These polyspecific/multispecific polypeptides can generally be as described in more detail in the International application PCT/BE2004/000159 for polyspecific polypeptides directed against EGFR and at least one further antigen/protein.

It has been demonstrated by the inventors (WO 04/041865) that (a) polypeptides comprising at least one Nanobody directed against a desired antigen and (b) at least one binding partner (such as at least one further Nanobody) directed against a serum protein of a subject, has significantly prolonged half-life in the circulation of said subject, compared with the half-life of the Nanobody directed against the desired antigen per se. Furthermore, such multispecific polypeptides were found to exhibit the same favourable properties of Nanobodies per se, as described above, such as, for example, high stability remaining intact in mice, extreme pH resistance, high temperature stability and high target specificity and affinity.

The serum protein may be any suitable protein found in the serum of a subject, or fragment thereof. In one aspect of the invention, the serum protein is any of serum albumin, serum immunoglobulins, thyroxine-binding protein, transferrin or fibrinogen. The subject may be, for example, rabbit, goat, mouse, rat, cow, pig, camel, llama, monkey, donkey, guinea pig, chicken, sheep, dog, cat, horse, and preferably human. Depending on the intended use such as the required half-life for effective treatment and/or compartmentalization of the target antigen, the at least one further binding partner can be directed to one of the above serum proteins.

As mentioned above, the method of the present invention may be used for the cloning and direct screening of immunoglobulin sequences (including but not limited to multivalent polypeptides comprising: two or more variable domains—or antigen binding domains—and in particular V_{H} domains or V_{HH} domains; fragments of V_{L}, V_{H} or V_{HH} domains, such as CDR regions, for example CDR3 regions; antigen-binding fragments of conventional 4-chain antibodies such as Fab fragments and scFv's, heavy chain antibodies and domain antibodies; and in particular of V_{H} sequences, and more in particular of V_{HH} sequences) that can be used as part of and/or to construct such multispecific constructs.

Preferably, in another aspect of the invention, Nanobodies consisting of at least part of the heavy chain of a molecule from the Ig superfamily may be the end product of processes involving methods according to the present invention. Said at least part of the heavy chain of a molecule from the Ig superfamily can be (part of) a CDR region, preferably (part of) a CDR3 region. Said end product Nanobodies or fragments thereof may be monovalent, bivalent, or multivalent in a higher order, or fused or combined with other polypeptides to form bispecific or multispecific polypeptides.

The present invention describes a method for the cloning and production of variable antibody domains (or antigen binding domains), heavy chain variable domains, light chain variable domains, CDR regions, CDR3 regions, smaller antigen binding fragments (Fab fragments and scFv), antibodies devoid of light chains, Camelidae heavy chain antibodies, V_{HH} domains (or Nanobodies), camelised heavy chain antibodies, domain antibodies, and heavy chain antibodies.

The method of the present invention involves identifying within a large population of lymphoid cells a collection of lymphocytes that are producing an antibody with a desired specificity or function, and then rescuing from those lymphocytes the genetic information that encodes the specificity of the antibody. The method of the present invention further involves identifying within a large population of lymphoid cells a single lymphocyte that is producing an antibody with a desired specificity or function, and then rescuing from that lymphocyte the genetic information that encodes the specificity of the antibody. This method permits the production of the high-affinity antibodies generated during in vivo immune responses.

The method of the present invention allows identification of very high affinity antibodies with greater efficiency and reliability than traditional hybridoma methods and Ig libraries allow.

The method of the present invention allows to directly obtain therapeutic variable antibody domains comprising Nanobodies from antigen-specific B cells of immunized animals. This strategy is advantageous in that it is not only a rapid protocol, but it also allows the cloning of variable antibody domains existing even at a rare frequency.

As a limited example, therapeutic Nanobodies directly obtained by the method of the present invention also encompass bivalent (or multivalent) Nanobody-constructs, as well as bispecific (or multispecific) Nanobody-constructs.

An advantage of the method of the present invention over existing antibody selection processes, is that antibodies can often show high binding affinity but little functional effect. The method of the present invention allows screening simultaneously for both binding ability and functional characteristics, enabling identification of an individual B cell producing the optimal antibody.

A major advantage of the method of the present invention over existing antibody selection processes is the direct screening for multivalent antibody constructs. Examples of such constructs include, but are not limited to, bivalent constructs including two identical or non-identical antibodies linked to each other (with or without a linker sequence), bispecific constructs including an antibody against the target and a serum protein or any other substance to improve the half-life of the construct, or any other construct including an antibody against the target and another antibody, or another functional protein domain such as an enzyme or a receptor or a ligand.

The inventors have previously demonstrated that multivalent constructs may have enormously improved binding affinity compared to the monovalent construct for a specific target (e.g. WO 04/041862, WO 04/062551), especially for multimeric antigens or cell surface antigen. Existing antibody selection processes do not provide information on the potency increase of multivalent constructs. Existing antibody selection processes have a tendency for the selection of avid antibodies, but during the screening phase only use monovalent antibody fragments for selecting the antigen binding characteristics. Reformatting of individual lead fragments into bivalent constructs is a cumbersome process, based on trial-and-error and with low success rate.

According to the method of the present invention, individual clones can directly be expressed as bivalent (or multivalent) constructs, or as enzyme fusions, or fused on membrane. The method of the present invention allows for an adaptable direct screening to any application without the bias of a selection system (related to for instance the multimeric display observed with phage or other display systems). This is a great improvement over existing antibody selection and screening methods.

The method is applicable for cloning a complete form of the immunoglobulin genes and is useful for analyzing immune responses. The method can be used to analyze the response to several forms of antigen containing the same peptide e.g. the free peptide form, the whole peptide, the peptide coupled to a carrier protein, etc.

A further advantage of the method of the present invention over existing antibody selection processes, is that the scope of these latter is limited to the selection of proteins and furthermore does not allow direct selection for activities other than binding, for example catalytic or regulatory activity. The method of the present invention allows to directly screen for binding ability, functional characteristics, biological activity and even rare B cell activities, enabling to identify an individual B cell producing the optimal antibody.

Method

According to an aspect of the present invention, there is provided a method of cloning a nucleic acid having a nucleotide sequence which encodes an immunoglobulin against at least one antigen or which encodes at least one part of an immunoglobulin directed against at least one antigen, comprising the steps of:
a) obtaining B-lymphocytes from at least one animal immunized with said at least one antigen or from at least one non-immune animal immunized in vitro with said at least one antigen,
b) selecting from said B-lymphocytes at least one B-lymphocyte with specificity against said at least one antigen,
c) obtaining a nucleic acid from said at least one B-lymphocyte selected in step (b), wherein said nucleic acid encodes said immunoglobulin against said at least one antigen or encodes at least one part of said immunoglobulin directed against said least one antigen,
d) amplifying and/or cloning said nucleic acid so as to obtain an amplified and/or cloned nucleic acid which encodes said immunoglobulin against said at least one antigen or which encodes at least one part of said immunoglobulin directed against said at least one antigen.

Preferably, in said method, said at least one part of an immunoglobulin directed against at least one antigen may be an immunoglobulin variable region.

Even more preferably, in said method, said at least one part of an immunoglobulin directed against at least one antigen may be an immunoglobulin heavy chain variable region (V_{H} domain), i.e. from a suitable animal, and in particular a mammal, and more in particular a human being.

In particular, in said method, said at least one part of an immunoglobulin directed against at least one antigen may be an immunoglobulin heavy chain variable region from a heavy chain antibody (V_{HH} domain). In this aspect, animal immunized with said at least one antigen is a species of Camelid, such as a camel, dromedary or llama.

In the above method, step (b) is preferably performed by screening said B-lymphocytes for affinity against said at least one antigen, and selecting at least one B-lymphocyte with affinity for said at least one antigen.

More preferably, in the above method, step (b) is performed by panning the B-lymphocytes against the at least one antigen immobilized on a solid support, and selecting at least one B-lymphocyte with affinity for said at least one immobilized antigen.

Also, in step (c) of the above method, the nucleic acid that is obtained from said at least one B-lymphocyte is preferably mRNA. Prior to step (d), said mRNA is converted into cDNA in a manner known per se.

The amplification and cloning in step (d) is preferably performed by:

(d1) an amplification reaction using a first primer capable of hybridizing to a poly-A site located distal to the 3′-end of framework 4 region, and a second primer capable of hybridizing to a site at or adjacent to the 5′-end of framework 1 region, so as to produce nucleic acid comprising at least part of the variable domain immunoglobulin sequences,

(d2) cleaving the double stranded DNA at a naturally-occurring restriction enzyme site, positioned such that cleavage with the restriction enzyme directed thereto produces double stranded DNA encoding a functional variable domain immunoglobulin, and

(d3) cloning the double stranded DNA thus obtained into a suitable vector.

Said amplification reaction in step (d1) is preferably performed by PCR. Also, the first primer used in the amplification reaction in step (d1) preferably comprises the sequence oligo-dT.

In step (d2), the double stranded DNA is preferably cleaved at a BstEII restriction enzyme site.

The second primer used in the amplification reaction in step (d1) preferably has a nucleotide sequence that encodes for at least one enzyme restriction site.

Also in step (d3), the double stranded DNA obtained in step (d2) is preferably cloned into a non-expression vector.

The above method may also comprise the further steps of
e) isolating, from the vector obtained in step (d3), a nucleic acid having a nucleotide that encodes said immunoglobulin against said at least one antigen or which encodes at least one part of said immunoglobulin directed against said at least one antigen, wherein said nucleic acid comprises the variable domain of said immunoglobulin directed against said at least one antigen or at least part thereof; and
f) cloning said variable domain of said immunoglobulin directed against said at least one antigen or at least part thereof into a suitable vector.

If so, the vector used in step (f) is preferably different from the vector used in step (d3), and is more preferably an expression vector.

The cloning of step (f) may comprise the use of a gene swapping system, for example using vector pAX056a as an entry vector and vector pAX056b a destination vector. Said gene swapping system may for example be a gene swapping system that involves the use of recombinase. All this will be clear to the skilled person.

To express the immunoglobulin (or a part of fragment thereof) as a multivalent (and usually bivalent construct) the vector used in step (f) may be such that, following expression of the variable domain of said immunoglobulin directed against said at least one antigen or at least part thereof, the variable domain of said immunoglobulin directed against said at least one antigen or at least part thereof is obtained as a bivalent construct. The construction, cloning and expression of such a construct will be clear to the skilled person based on the further disclosure herein. For example, in step (f), two copies of the nucleotide sequence encoding said variable domain of said immunoglobulin directed against said at least one antigen or at least part thereof are cloned into said vector, in such a way as to provide a vector that, following expression, provides a bivalent construct comprising a fusion of two linked variable domains of said immunoglobulin directed against said at least one antigen, or at least two parts thereof, optionally linked via a linker sequence.

The invention also relates to a nucleotide sequence and/or nucleic acid which encodes an immunoglobulin against at least one antigen or which encodes at least one part of an immunoglobulin directed against at least one antigen, obtained by the above method.

Said nucleotide sequence and/or nucleic acid preferably encodes a mammalian immunoglobulin against said at least one antigen or which encodes at least one part of a mammalian immunoglobulin directed against said at least one antigen, and in particular a human immunoglobulin against said at least one antigen or which encodes at least one part of a human immunoglobulin directed against said at least one antigen. More preferably, said nucleotide sequence and/or nucleic acid encodes at least a V_{H} domain of an immunoglobulin directed against said at least one antigen.

According to another preferred embodiment, said nucleotide sequence and/or nucleic acid encodes a heavy chain antibody against said at least one antigen or which encodes at least one part of a heavy chain antibody directed against said at least one antigen. More preferably, said nucleotide sequence and/or nucleic acid encodes at least a V_{HH} domain of a heavy chain antibody directed against said at least one antigen.

The above nucleotide sequence and/or nucleic acid is preferably in the form of DNA, and in particular in the form of double stranded DNA. The nucleotide sequence and/or nucleic acid may for example be in the form of a vector, such as an expression vector or a non-expression vector. The nucleotide sequence and/or nucleic acid may also be in the form of a genetic construct.

In one embodiment, for the expression of a multivalent construct, the nucleotide sequence and/or nucleic acid is preferably in the form of a genetic construct that is such that, upon expression in a suitable manner and using a suitable expression system, it provides a multivalent polypeptide comprising at least two immunoglobulins against said at least one antigen, or at least two parts of said immunoglobulin against said at least one antigen.

The invention also relates to a method for obtaining an immunoglobulin against at least one antigen or which encodes at least one part of an immunoglobulin directed against at least one antigen, said method comprising the expression, in a suitable manner and using a suitable expression system, of a nucleotide sequence or nucleic acid as described above. The invention also relates to an immunoglobulin against at least one antigen or which encodes at least one part of an immunoglobulin directed against at least one antigen, obtained by said method. Preferably, said immunoglobulin comprises at least a V_{H} domain of an immunoglobulin directed against said at least one antigen, or comprises at least a V_{HH} domain of a heavy chain antibody directed against said at least one antigen.

The invention also relates to polypeptides that comprise at least one such immunoglobulin and at least one further amino acid sequence. For example, such a polypeptide may be a multivalent or multispecific polypeptide, e.g. comprising at least two V_{H} domains of an immunoglobulin directed against said at least one antigen, or comprising at least two V_{HH} domains of a heavy chain antibody directed against said at least one antigen.

The above method may include a further step of separating a single clone from the non-expression vector obtained in step (f), and of cloning therefrom the nucleic acid encoding a variable domain immunoglobulin into an expression vector allowing the expression of monovalent or bivalent format (or higher orders of valency constructs), or fused to enzymes or other protein domains.

Obtaining B-lymphocytes

In one step of the method, B-lymphocytes are obtained from an animal capable of producing immunoglobulins naturally devoid of light chains, which has been immunised with antigen of interest. Such immunization and B-lymphocyte preparation methods are known in the art. Antigen may be any substance of interest capable of eliciting an immune response. Antigens include, but are not limited to proteins, peptides, glycoproteins, polysaccharides, nucleic acid, synthetic polymers, small organic molecules, combinations of two or more of the aforementioned substances.

Methods of obtaining B-lymphocytes are known in the art, and a specific embodiment is provided in the Examples section.

According to one embodiment of the invention, peripheral blood mononuclear cells (PBMC) are prepared from the purified blood and optionally the tissues of the animal after the final immunisation. Methods for obtaining PBMC are known in the art. According to one aspect, PBMC are obtained by centrifuging blood of the animal on a Ficoll Paque™ PLUS density gradient (Amersham Biosciences). According to another embodiment of the invention, B-lymphocytes are recovered from PBMC. Such recovery may include the steps of lysing erythrocytes, depleting monocytes from the PBMC and using the resultant supernatant which contains B-lymphocytes, labeling B-cells using monoclonal antibodies or polyclonal antisera and sorting B-cells using flow cytometry and/or immunomagnetically and/or density altering particles, labeling all cells but B-cells using monoclonal antibodies or polyclonal antisera and sorting the non-labelled cells using flow cytometry and/or immunomagnetically and/or density altering-particles.

Immunisation

Many warm-blooded animals, such as humans, rabbits, mice, rats, sheep, cows or pigs may be immunized in order to obtain antibody-forming cells. However, mice and rats are generally preferred because of their ease in handling, well-defined genetic traits, and the fact that they may be readily sacrificed. For the use of Nanobodies as described in the present invention, llama are used for obtaining antibody-forming cells.

Procedures for immunizing animals are well known in the art. Briefly, animals are injected with the selected antigen against which it is desired to raise antibodies. Then, selected antigen may be accompanied by an adjuvant or hapten, as discussed above, in order to further increase the immune response. Usually the substance is injected into the peritoneal cavity, beneath the skin, or into the muscles or bloodstream. The injection is repeated at varying intervals and the immune response is usually monitored by detecting antibodies in the serum using an appropriate assay that detects the properties of the desired antibody. Large numbers of antibody-forming cells can be found in the spleen and lymph nodes of the immunized animal. Thus, once an immune response has been generated, the animal is sacrificed, the spleen and lymph nodes are removed and a single cell suspension is prepared using techniques well known in the art. Alternatively, the circulating lymphocytes are obtained from the blood of the animal. Antibody-forming cells may also be obtained from a subject which has generated the cells during the course of a selected disease. For instance, antibody-forming cells from a human with a disease of unknown cause, such as rheumatoid arthritis, may be obtained and used in an effort to identify antibodies which have an effect on the disease process or which may lead to identification of an etiological agent or body component that is involved in the cause of the disease. Similarly, antibody-forming cells may be obtained from subjects with disease due to known etiological agents such as malaria or AIDS. These antibody-forming cells may be derived from the blood or lymph nodes, as well as from other diseased or normal tissues. Antibody-forming cells may be prepared from blood collected with an anticoagulant such as heparin, citrate or EDTA. The antibody-forming cells may be further separated from erythrocytes and polymorphonuclear cells using standard procedures such as centrifugation with Ficoll-Hypaque PLUS (Amersham Biosciences, Uppsala, Sweden). Antibody forming cells may also be prepared from solid tissues such as lymph nodes or tumors by dissociation with enzymes such as collagenase and trypsin in the presence of EDTA.

Antibody forming cells may also be obtained by culture techniques such as in vitro immunization. Examples of such methods are known in the art.

Obtaining Antigen-Specific B-Lymphocytes

According to a subsequent step of the invention, B-lymphocytes obtained from the blood and optionally tissues of the antigen-immunised animal are selected for antigen-binding capability. A sample collection of B-lymphocytes of an immunized animal comprises a diversity of antibodies, some of which will bind the antigen, and others not.

In one embodiment the sample collection comprises a single antigen-specific B-lymphocyte.

Employing a B-lymphocyte selection method based on cells binding to the antigen of interest through their membrane-bound immunoglobulins or via an affinity matrix, permits one to obtain a set of B-cells whose DNA and mRNA codes for immunoglobulins binding the antigen of interest. The set of B-cells so obtained may comprise a specific heterogeneous mix of anti-antigen antibodies of different affinities and epitope specificities.

Selection of antigen-specific B-lymphocytes may be performed according to any method of the art. Specific embodiments are provided in the Examples.

According to one embodiment of the invention, antigen-specific B-lymphocytes are obtained by panning B-lymphocytes in tubes, flasks or plates coated with antigen. According to one embodiment of the invention, antigen-specific B-lymphocytes are obtained by panning B-lymphocytes using magnetic microbeads coated with antigen (e.g. Dynal beads or MACS). Surface- or bead-bound B-cells may be removed from the beads or surface by enzymatic treatment such as trypsin or other proteases, addition of bivalent cation chelating agents such as EDTA to the medium, addition of agents breaking down the physical link between antigen and carrier or surface such as DTT when reduceable linkers were used, competitive displacement with another antigen binding ligand, or combinations thereof. In case small particulate carriers such as magnetic microbeads are used, the carriers may be left attached to the carrier binding B-cells after separation of carrier binding and non-binding B-cells. According to one embodiment of the invention, secreted immunoglobulin is captured on the cell membrane of the originating B-cell via an affinity matrix, consisting of a B-cell binding moiety, such as anti-CD19 or anti-CD45 and an immunoglobulin binding moiety, such as anti-llama immunoglobulin Fc. According to one embodiment of the invention, secreted immunoglobulin is retained near the originating B-cell via an semi-solid affinity matrix surrounding the cell, such as used in gel microdroplet encapsulation methods.

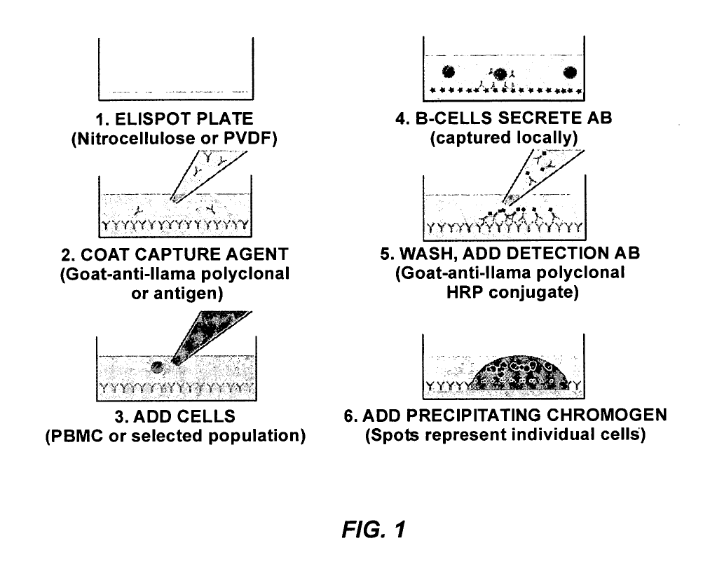

Optionally, captured B-lymphocytes may be assayed to check whether they bind to antigen. Such assay may be any of those known in the art. An example of an assay includes the use of flow cytometry using fluorescently labelled antigen or ELISPOT immunoglobulin secretion assays using goat-anti-llama polyclonal antibody or antigen immobilised on a solid phase such as nitrocellulose or PVDF. B-lymphocytes are brought into contact with the coated membrane and allowed to secrete antibody. Said secreted antibodies become immobilized on the solid phase and can be detected, for example, using goat-anti-llama polyclonal HRP conjugate and chromogen (see FIG. 1).

Separation of 4-Chain and Heavy Chain Antibody Producing B-cells

Optionally, a step of the invention may be to separate 4-chain antibody producing B-cells from heavy chain antibody producing B-cells prior to or after selection of antigen-binding B-cells. This may be accomplished by the use of heavy chain antibody specific reagents to label the membrane-bound immunoglobulin present on the B-cells (as a positive marker) or conventional antibody specific reagents (as a negative marker). Said reagents may include, but are not limited to, polyclonal or monoclonal antibodies generated against V_{HH}s preferentially binding heavy chain antibodies, polyclonal or monoclonal reagents selectively binding the heavy chain of either heavy chain antibodies (positive marker) or 4-chain antibodies (negative marker), or binding the light chain present only in 4-chain antibodies (negative marker). Methods to prepare and characterize such polyclonal or monoclonal antisera are well known to those skilled in the art. Non-limiting examples of reagents suitable for detection of such positive and negative markers as described above are given in Daley et al. (Clin Diagn Lab Immunol. 2005 12: 380) and US2002/0155604 A1, respectively.

Separation of 4-chain antibody producing B-cells and heavy chain antibody producing B-cells using such reagents can be accomplished using panning techniques whereby the selective reagent is coated onto a solid support, such as plasticware or magnetic beads, or by fluorescently labeling the selective reagents and flow cytometrically sorting out heavy chain antibody displaying B-cells based on positive and/or negative gating, depending on the type of reagent used for staining the mixture of cells. Due to the multiparametric measurements possible in modern cell sorters, sorting of heavy chain producing B-cells can conveniently be combined with positive selection for fluorescently labeled antigen binding, negative selection of dead cells; positive selection for the presence of other B-cell or negative selection of non-B-cell markers (such as those described by Davis et al., Vet Immunol Immunopathol. 2000 74: 103) expressing cells etc.

In Vitro Stimulation of B-cells

Optionally, a step of the invention may be to stimulate B-cells in vitro prior to or after selection of antigen-binding B-cells or separation of heavy chain or 4-chain immunoglobulin producing B-cells. B-cell stimulation results in production of more immunoglobulin mRNA per cell, division of the cell leading to clonal expansion and enhanced production of soluble immunoglobulin which is released into the medium. Increases in mRNA content and clonal expansion both simplify the recovery of the immunoglobulin encoding mRNA sequences by RT-PCR or similar methods, as more template is available for amplification.

Various methods for effective in vitro stimulation of B-cells have been described, all of which may be effective for stimulating Camelid B-cells. Zubler and co-workers (Wen et al., Eur J. Immunol. 1987 17: 887) described the use of mutant EL4 subclone, EL4-B5 as stimulator/feeder cells in B-cell cultures. Banchereau and co-workers (Valle et al., Eur J Immunol. 1989 19: 1463) described the use of agonistic anti-CD40 monoclonals, displayed on Fc-gamma receptor expressing fibroblasts used as feeder cells. More recently, CD40L transfected cell lines have been used as stimulator/feeder cells (Armitage et al., Nature. 1992 357: 80 and Spriggs et al., J Exp Med. 1992 176: 1543) as well as recombinant soluble fragments of CD40L (Hollenbaugh et al., EMBO J. 1992 11: 4313 and Mazzei et al., J Biol. Chem. 1995 270: 7025).

Stimulation of B-cells in these systems also requires a source of exogenous growth factors. Cell culture medium previously conditioned by activated T-cells, from both the same species or xenogenic origin as compared to the Camelid B-cells, can be used as a convenient source of such factors. Recently, Odbileg et al. identified the cDNA sequence of many llama cytokines (Vet Immunol Immunopathol. 2004 102: 93 and Vet Immunol Immunopathol. 2005 104: 145), making it feasible to express llama cytokines as recombinant proteins and use defined-mixtures of purified growth factors in Camelid B-cell cultures as a substitute for poorly defined T cell conditioned media.

Screen of In Vitro Stimulated B-Cells

Release of soluble immunoglobulin into the medium by stimulated B-cells enables one to conveniently screen B-cell cultures for the presence or absence of antigen-specific heavy-chain antibodies. For instance, one can test the conditioned supernatant by removing the conditioned medium from the cells and use all or part of the sample in an immunoassay configured to quantify immunoglobulin concentrations present in the medium to reveal which stimulated cultures contain successfully stimulated B-cells. This enables one to exclude unsuccessfully stimulated B-cell cultures in subsequent steps of the immunoglobulin gene cloning procedure. Use of the same supernatant in an immunoassay configured to detect either only heavy-chain immunoglobulins or only 4-chain immunoglobulins will reveal which cultures contain stimulated B-cells of either type. This allows one to exclude 4-chain immunoglobulin producing B-cell clones from the subsequent cloning procedures, as only cloning of heavy chain immunoglobulins will result in functional Nanobodies. Using the B-cell conditioned supernatants in immunoassays configured to detect antigen binding immunoglobulin, using secondary detection reagents either specifically for heavy-chain immunoglobulins or 4-chain immunoglobulins, allows one to determine which wells contain stimulated B-cells encoding heavy chain immunoglobulins binding the antigen and which ones contain antigen binding but irrelevant (4-chain type) immunoglobulin producing B-cells. Again, use of such a screening assay allows one to focus the downstream cloning of immunoglobulin genes towards the only relevant B-cell clones (antigen specific, heavy chain immunoglobulin producing cells). The reagents required for heavy-chain or 4-chain immunoglobulin selective immunoassays are available and identical to those described above (“Separation of 4-chain and heavy chain antibody producing B-cells”).

Having access to stimulated B-cell conditioned supernatants also enables one to screen for B-cell clones producing immunoglobulin having desirable functional characteristics, such as being able to neutralize receptor/ligand interaction where either one is the antigen in question, having an agonistic or antagonistic effect on receptor activation when isolating Nanobodies having such an effect, having high antigen binding affinity or being able to inhibit enzymatic activity when trying to isolate enzyme activity inhibiting Nanobodies. Screening for such characteristics can be performed on antibody isolated from conditioned supernatants collected off the B-cell cultures, but usually can be performed more conveniently on the conditioned supernatant itself. Methods for screening antibody containing solutions such as B-cell conditioned supernatants for the type of activities mentioned above are known to those skilled in the art. Both heterogeneous methods (such as chromogenic, fluorescent or radioactive readout immunoassays in plates, on beads or microarrays and bioassays) as well as homogeneous assays (such as LANCE, Alphascreen or using confocal imaging systems such as ABI's FMAT or Evotech's Opera) are suitable for binding and activity assays. As methods for affinity determination, bioassays, surface plasmon resonance or cantilever MEMS based devices as well as off-rate selective immunoassays (Friguet et al., J Immunol Methods. 1985 77: 305) are mentioned as non-exclusive examples.

Having separated the conditioned supernatant from the corresponding B-cells prior to supernatant analysis, and having saved all B-cell cultures during the analyses on the supernatants, it is possible to retrieve all B-cells present in the original B-cell culture of only the most interesting wells and use these to rescue the V_{HH}-encoding mRNA sequence using the methods described as follows. B-cell pellets from which the conditioned supernatant has been removed for analysis can be stored in various ways during conditioned supernatant analysis: as intact frozen cells using media suitable for storing live mammalian cells (i.e. cell culture medium containing 10% DMSO), as frozen cell lysates prepared by lysing the cell pellets using an RNA protective cell lysis solution (i.e. TRIzol, Invitrogen) or in a buffer designed to protect RNA from degradation at room temperature or below without lysing the cells (i.e. RNAlater, Ambion).

Preparation of mRNA

According to a subsequent step of the invention, RNA is isolated from antigen specific B-lymphocytes. The RNA obtained is a collection of nucleic acids, already selected from the immune repertoire of the animal by B-lymphocyte panning, and contains mRNA's encoding immunoglobulin binding the antigen of interest. As such, the collection of mRNA comprises far less irrelevant (i.e. not directed against the target) immunoglobulin mRNA. Methods to isolate RNA are known in the art, and include TRIzol reagent (Invitrogen) and the Gough method (Gough, N. Mex., Anal. Biochem, 1988 173(1); 93-5).

Contrary to current perception, sufficient quantities of RNA can be obtained from antigen-specific B-lymphocytes, such that a heterogenous collection of antigen-specific Nanobodies becomes available (see Examples).

Direct Cell-to-Nanobody Amplicon Rescue Procedure

In order to produce recombinant Nanobodies, it is required to rescue the heavy chain immunoglobulin variable region encoding mRNA segment sequence of an individual antigen-specific heavy chain immunoglobulin producing B-cell or from a sample of genetically identical B-cells clonally expanded from a single in vitro stimulated B-cell. This procedure can be performed by sequentially isolating total RNA or mRNA from either type of sample, converting the mRNA into cDNA by reverse transcriptase next and finally amplifying the relevant gene segment using PCR or similar methods. Methods to perform these discrete steps are detailed below.

However, it is possible to perform the first two steps or all three steps in a single tube format, that is, without isolation of RNA or mRNA from the cell or cells of interest. It is well known in the art that an individual cell or multiple cells can be deposited directly into vessels containing a reaction mixture of reverse transcriptase enzyme and primers in a suitable buffer, using a cell sorter or micromanipulation device, and obtain cDNA suited as template for PCR amplification. This PCR amplification can be performed in another tube, using all or a portion of the cDNA generated in the first reaction (“two-step RT-PCR”). Furthermore, it is also possible to combine the reverse transcription reaction performed directly on cells with PCR amplification of the resulting cDNA in the same tube, in one continuous series of reactions (“one-step RT-PCR”). Convenient and validated kits for such combined procedures can readily be obtained from many suppliers, two examples of which are Cells-to-signal (Ambion) and Superscript III One-step RT-PCR System (Invitrogen). Primers needed for cDNA synthesis and PCR amplification in such one-step or two-step RT-PCR systems are identical to those described below for the consecutive steps of a procedure consisting of separate RNA isolation, cDNA synthesis and PCR amplification steps.

Synthesis of cDNA

According to a subsequent step of the invention, single stranded cDNA is synthesized from mRNA isolated from antigen-specific B-lymphocytes. Double stranded DNA may subsequently be prepared from the single stranded cDNA. Methods for the preparation of cDNA and double stranded DNA are known in the art; a specific embodiment is provided in the Examples section.

According to one embodiment of the present invention, single stranded cDNA is prepared using an oligo-dT primer or random primer and a reverse transcription (RT) reaction. The RT reaction may be performed using known methods, such as, for example, using a reverse transcriptase enzyme or a ready-made kit therefor. The DNA-RNA hybrid product of reverse transcription may be treated with an enzyme to remove the RNA e.g. RNAseH. The single stranded cDNA so formed may be purified.

Nanobody Amplification

According to a subsequent step of the invention, a collection of Nanobody genes is amplified from the cDNA in one amplification reaction for cloning into a non-expression vector. Said collection does not represent a sample repertoire of the animal, but is instead focused on a narrow, heterogeneous mix of immunoglobulins only directed towards the antigen.

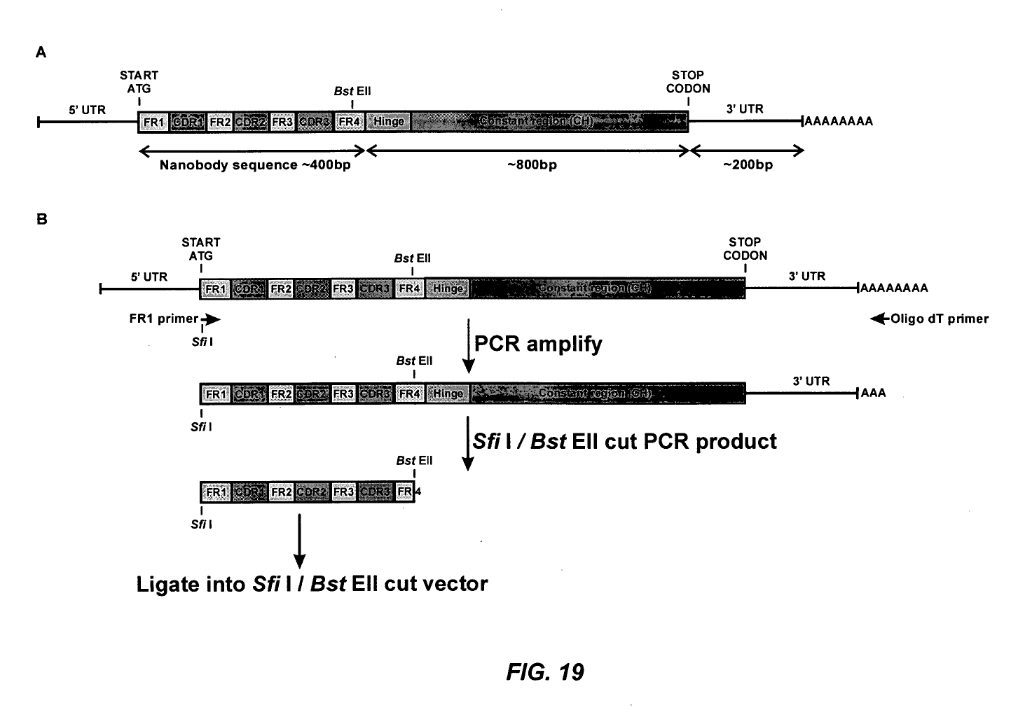

The inventors have found that the amplification should be performed using a universal 3′ end primer having a sequence oligo-dT and a framework-specific 5′ end primer also known as FR1 primer herein (FIG. 19B). The use of an oligo dT primer increases the diversity of genes in the collection, since it reduces the dependency of variations in sequences for annealing of the oligo nucleotide primer, which can be caused by differences in isotypes, haplotypes or mutations introduced by somatic mutation process. The oligo dT primer also permits utilisation of a BstEII restriction site which naturally occurs at the 3′ end of framework 4 (FR4) for cloning, precluding the necessity for introducing a 3′ restriction site during the reverse transcription or PCR reaction which could affect the efficient hybridization of the primer to its template and thereby reduce the annealing efficiency of such an oligo.

The oligo-dT primer anneals to a site distal from the end of the FR4 region. Such location can be between 100 and 200 nucleotides away from the C-terminal end of the immunoglobulin encoding cDNA sequence, according to the findings of the inventors (FIG. 19A). It would be expected that the additional, non-coding DNA such broader annealing entails would cause errors in DNA synthesis or strongly reduced amplification efficiency, owing to the increased size of the product. However, the use of an annealing site not at or adjacent to the 3″ end of the light-chain devoid immunoglobulin heavy chain gene containing the Nanobody gene segment surprisingly provides higher diversity of antigen-specific Nanobodies as compared to amplifications using adjacent primers. Thus, contrary to expectation such distally located poly-A priming site provides better results than would be expected by those skilled in the art.

Other suitable amplification techniques and primers will be clear to the skilled person, and for example include the method and primers described in the European patent 0 368 684.

The framework specific FR1 primer anneals within or in the leader sequence just upstream of the FR1 region (FIG. 19B). According to one aspect of the invention, the framework-specific primer anneals to at least the first nucleotide of FR1. According to another aspect of the invention, the framework-specific primer permits amplification of at least the first nucleotide of FR1. According to another aspect of the invention, the framework-specific primer anneals within FR1. Examples of 5′-end primers incorporating a SfiI and NcoI are provided in Table 8 below:

TABLE 8

Examples of 5′-end primers incorporating
a SfiI (bold) and NcoI (underlined)
restriction site.

SEQ
ID

| | |
|---|---|
| NO | Sequence (5′-3′) |

| | |
|---|---|
| 1 | CATGCCATGACTCGCGGCCCAGCCGGCCATGGCCGAGGTGCAGCTGG |
| | TGGAGTCTGG |
| 2 | CATGCCATGACTCGCGGCCCAGCCGGCCATGGCCGATGTGCAGCTGG |
| | TGGAGTCTGG |
| 3 | CATGCCATGACTCGCGGCCCAGCCGGCCATGGCCGCGGTGCAGCTGG |
| | TGGAGTCTGG |
| 4 | CATGCCATGACTCGCGGCCCAGCCGGCCATGGCCGCCGTGCAGCTGG |
| | TGGATTCTGG |
| 5 | CATGCCATGACTCGCGGCCCAGCCGGCCATGGCCCAGGTGCAGCTGG |
| | TGGAGTCTGG |
| 6 | CATGCCATGACTCGCGGCCCAGCCGGCCATGGCCCAGGTACAGCTGG |
| | TGGAGTCTGG |
| 7 | CATGCCATGACTCGCGGCCCAGCCGGCCATGGCCCAGGTAAAGCTGG |
| | AGGAGTCTGG |

A unique restriction site is found to be present close to the 3′-end in the heavy chain variable region of antibodies from human or other mammals (FIGS. 19A and 19B). It is generally located close to the 3′ end of the FR4 region. Such restriction site permits cloning of the amplified Nanobody gene without the need for a restriction site in the 3′-end primer. According to one embodiment of the invention, the unique restriction site is BstEII. Once obtained, the amplified DNA may be cloned into a vector not capable of expression in order to obtain a stable collection of antigen-specific Nanobody genes, preferably for storage.

As an alternative, primers can be used that allow for direct expression of the Nanobody gene in a cell-free protein expression system (also known as in vitro translation) without the need of cloning and transformation into a suitable host. These primers may add one or more of the following regulatory elements to the 3′ or 5′ end of the Nanobody gene: promoter and terminator sequences, ribosome binding site sequences (e.g., Kozak sequences, Shine-Dalgarno sequences) and translation initiation and termination codons.

Cloning Into a Non-Expression Vector

It is an aspect of the invention that the collection of antigen specific Nanobody genes is subsequently cloned into vectors which are not capable of providing protein expression in cell-free systems, prokaryotic or eukaryotic host cells. A non-expression vector according to the invention is any vector incapable of expressing the gene cloned therein as protein. Such vectors are well known in the art. Specific embodiments of cloning into a non-expression vector are provided in the Examples section. The use of non-expression vectors provides clones which are more stably stored because cellular aberrations resulting from leaky-expression vectors do not have the opportunity to arise. Thus, collections of genes encoding Nanobodies directed against an antigen can be stored for prolonged periods. The inventors have found the use of a non-expression vector can also increase the size and diversity of the collection so obtained.

Cloning Into an Expression Vector

According to another embodiment of the invention, when expression of a Nanobody gene is required, a single, non-expression vector comprising a Nanobody gene is separated from the above mentioned collection, and the Nanobody gene therein is transferred to an expression vector. As such, the result is an expression vector cloned with a Nanobody gene directed towards the expression of one Nanobody. Means to perform such steps and vectors therefor are well known the art. Cloning methods include restriction and ligation of the gene or gene swapping. A specific embodiment is provided in the Examples section.

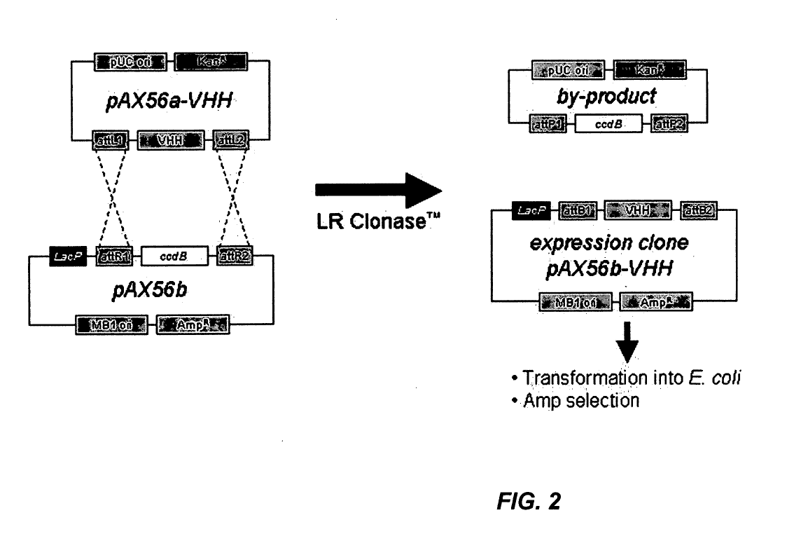

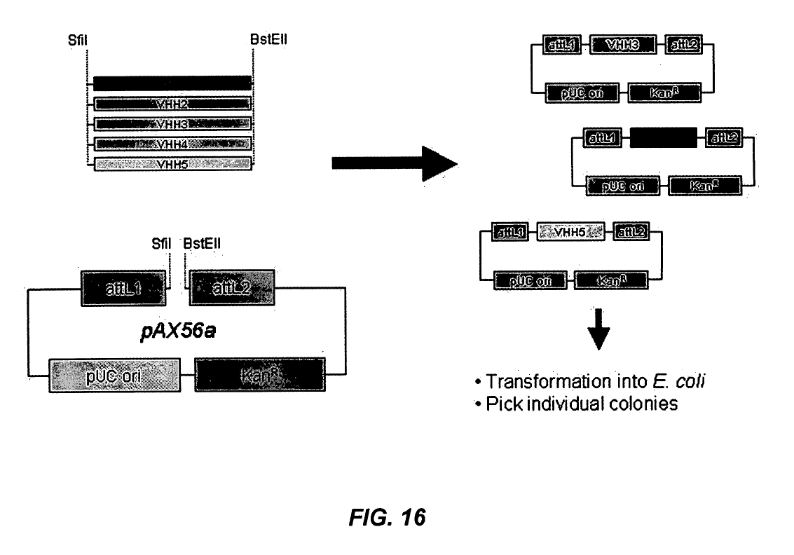

According to one aspect of the invention, the separated non-expression vector in which the Nanobody clone of interest is present is part of a cloning system permitting transfer of the Nanobody gene using a recombinase enzyme. The system relies on nucleic acid sequences flanking the gene cloned into the non-expression vector and on the presence of reciprocating, sequences in the expression vector (FIG. 16). The transfer of the gene is facilitated by a recombinase enzyme. Such system permits convenient transfer of the cloned gene into an expression vector as and when required. Examples of such non-expression/expression vectors systems include pAX056a/pAX056b (FIG. 2), the Gateway® system (Invitrogen), and the Cre-lox BD Creator™ system (BD Biosciences). Other such systems are within the scope of the invention. By using such recombinase systems, the transfer of individual Nanobody encoding genes can be performed in high throughput format, but more importantly in bivalent (multivalent) or bispecific (multispecific) format, which are genetically fused and which can otherwise not be obtained in a feasible way.

In Vitro Assaying

The collection of Nanobody genes cloned into non-expression vectors is highly enriched for antigen-specific Nanobodies due to the enrichment of B lymphocytes via B-lymphocyte panning on antigen. Thus, it is not necessary to assay for binding. However, should confirmation be necessary, a Nanobody gene, once separated from the collection of non-expression vectors and cloned into an expression vector, may be expressed and assayed to determine the binding affinity for the antigen. Such assays are well known in the art and include ELISA, other antigen-immobilised solid phase assays or homogenous binding assays.

The inventors have found that by pre-selecting suitable immunoglobulins at the B-lymphocyte stage, not only are more diverse higher-affinity antibodies found, but also the screening of monovalent Nanobodies after cloning is avoided. Rather, each Nanobody can be screened individually in the format optimal for the (intended) therapeutic application. Thus, the method saves time and obviates the need to invest in expensive high-throughput automation or array-type screening technology, but more importantly allows the screening of the Nanobody in its optimal format for therapeutic use, which often is a multivalent construct. The Nanobody products from these small scale expressions can directly be screened in bioassays or receptor binding assays to identify the most potent lead molecules.

Further Expression and Tests

The Nanobody candidates (leads) may be sequenced and nucleotides corresponding thereto may be used in a vector suitable for large scale Nanobody production. Once sufficient quantities of Nanobody have been obtained, each Nanobody may be tested in further assays such as stability, affinity and toxicity.

Modifications

According to a further embodiment of the present invention, the sequence information and biochemical data of a Nanobody obtained according to the invention is used to generate Nanobodies with artificial (i.e. not naturally occurring) sequences.

Modifications to the sequence include optimisation of the codon usage of the DNA sequence, optimisation of binding site, and humanisation of the sequence.

Modifications as described above are known in the art.

### EXAMPLES

### Example 1

### Immunisation of Llama with EGFR Antigen

After approval of the Ethical Committee of the Faculty of Veterinary Medicine (University Ghent, Belgium), 4 llamas (024, 025, 026 and 027) were immunized with the epidermal growth factor receptor (EGFR) according to all current animal welfare regulations. To generate an antibody dependent immune response, two animals (024 and 025) were injected with intact human epidermoid carcinoma cells (A431; ATCC CRL 1555; Giard D J, Aaronson S A, Todaro G J, Arnstein P, Kersey J H, Dosik H, Parks WP 1973. In vitro cultivation of human tumors: establishment of cell lines derived from a series of solid tumors. J. Natl. Cancer Inst. 51:1417-1423), expressing approximately 1.2×10⁶ EGFR molecules on its cell surface, while A431 derived membrane extracts were administered to two other llamas (026 and 027). Each animal received seven doses of subcutaneously administered antigens at weekly intervals (Table 9).

TABLE 9
Immunization schedule and tissue collections of immunized llamas.

| Day | Llama 024 | Llama 025 | Llama 026 | Llama 027 |
|---|---|---|---|---|
| 0 | intact cells | intact cells | Vesicles | vesicles |
| 7 | intact cells | intact cells | Vesicles | vesicles |
| 14 | intact cells | intact cells | Vesicles | vesicles |
| 21 | intact cells | intact cells | Vesicles | vesicles |
| 28 | intact cells | intact cells | Vesicles | vesicles |
| 35 | intact cells | intact cells | Vesicles | vesicles |
| 42 | intact cells | intact cells | Vesicles | vesicles |
| 46 | 150 ml blood sample | 150 ml blood sample | 150 ml blood sample | 150 ml blood |
| | (PBL1) | (PBL1) | (PBL1) | sample (PBL1) |
| | | | lymph node bow | lymph node bow |
| 47 | lymph node ileum | | | |
| | lymph node bow | | | |
| | spleen | | | |
| | bone marrow | | | |
| 49 | | purified EGFR | 150 ml blood sample | 150 ml blood |
| | | | (PBL2) | sample (PBL2) |
| 55 | | purified EGFR | | |
| 59 | | 150 ml blood | | |
| | | sample (PBL2) | | |
| 60 | | lymph node ileum | | |
| | | lymph node bow | | |
| | | spleen | | |
| | | bone marrow | | |

When immunizing with intact cells, each dose consisted of 10⁸ freshly harvested A431 cells. The dose for immunization with membrane extracts consisted of vesicles prepared from 10⁸ A431 cells. Vesicles were prepared according to Cohen and colleagues (Cohen S, Ushiro H, Stoscheck C, Chinkers M, 1982. A native 170,000 epidermal growth factor receptor-kinase complex from shed plasma membrane vesicles. J. Biol. Chem. 257:1523-31). Vesicles were stored at −80° C. before administration. Two extra injections of eight microgram purified EGFR (Sigma) in an emulsion with the adjuvant Stimune (CEDI Diagnostics B.V.) were administered intramuscularly to llama 025 (Table 1).

### Example 2

### Evaluation of Induced Immune Responses in Llama

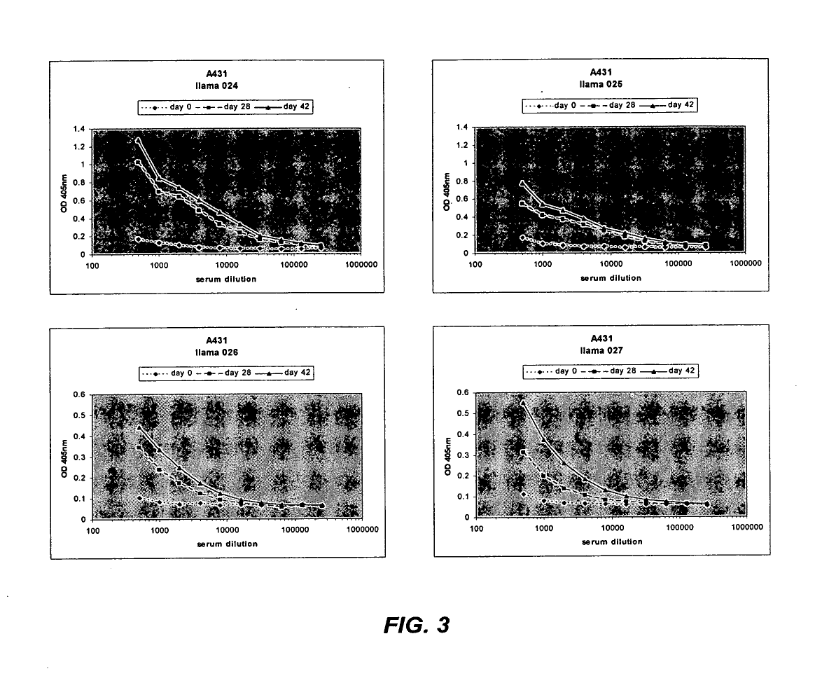

At day 0, 28 and 42, 10 ml of (pre-)immune blood was collected and serum was used to evaluate the induction of the immune responses in the 4 animals. A first ELISA was performed to verify whether the animals generated antibodies that recognized epitopes present on A431 cells. After coating a tissue-culture treated 96-well plate with 0.5% (w/v) gelatin in PBS (150 mM NaCl; 50 mM Na-phosphate, pH 7.4) for 10 minutes, the excess of gelatin was removed and A431 cells were grown overnight in the wells to confluency. Cells were fixed with 4% paraformaldehyde in PBS for 30 minutes at room temperature. Subsequently, the fixative was blocked with 100 mM glycine in PBS for 10 minutes, followed by blocking of the wells with a 4% skimmed milk-PBS solution, again for 10 minutes. Serum dilutions of immunized animals were applied and A431 specific antibodies were detected with a polyclonal anti-llama antiserum developed in rabbit, followed by a secondary goat anti-rabbit-horse radish peroxidase (HRP) conjugate (Dako). As substrate, 36 microliters of a 35% H₂O₂-solution in 21 ml ABTS 2,2-azino-bis(3-ethylbenz-thiazoline-6-sulfonic acid)-buffer was applied. The ABTS buffer consisted of 222.22 mg ABTS per liter of a 50 mM citrate, pH 4 solution. The colorimetric reaction was spectrophotometrically quantified as optical density (OD) at 405 nm. For all four animals, immunization with intact cells or membrane vesicles resulted in the induction of a significant A431-reactive antibody titer (FIG. 3).

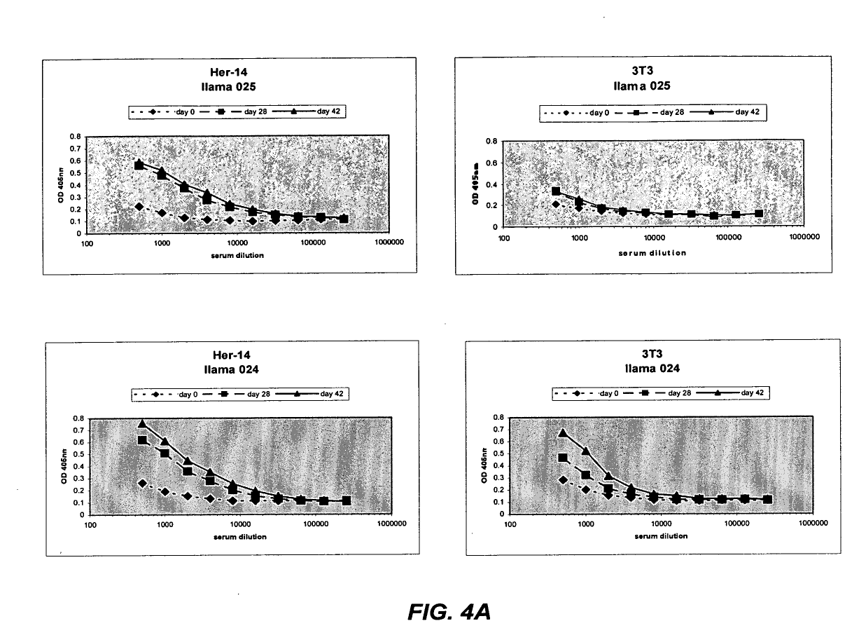

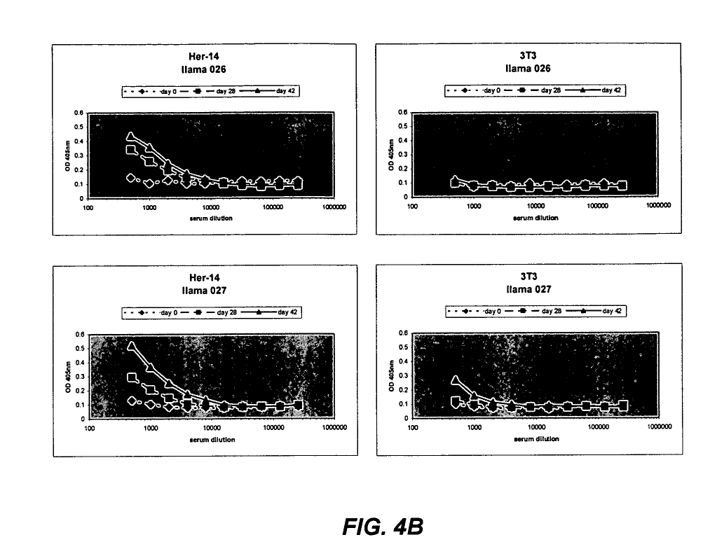

To verify whether the induced llama antibodies were EGFR specific, antibody titers in serum were evaluated on mouse fibroblasts stably expressing the human EGFR, Her14 (Honegger A M, Dull T J, Felder S, Van Obberghen E, Bellot F, Szapary D, Schmidt A, Ullrich A, Schlessinger J, 1987. Point mutation at the ATP binding site of EGF receptor abolishes protein-tyrosine kinase activity and alters cellular routing. Cell 51:199-209) versus the parental mouse fibroblasts cell line NIH3T3 clone 2.2 (3T3), similarly performed as described above. Again, the serum titer of antibodies reactive with Her14 cells was higher compared to the titer for the parental 3T3 cells, indicating that after immunization, circulating serum antibodies were EGFR specific (FIG. 4A for llama 024 and 025; FIG. 4B for llama 026 and 027).

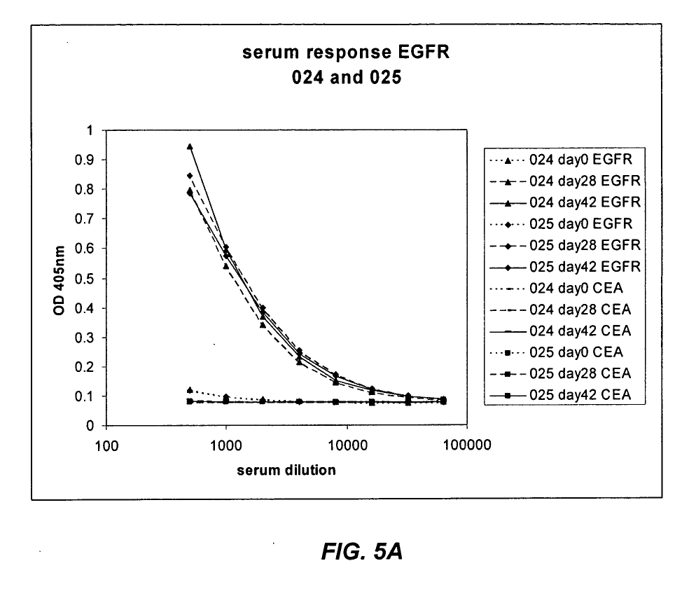

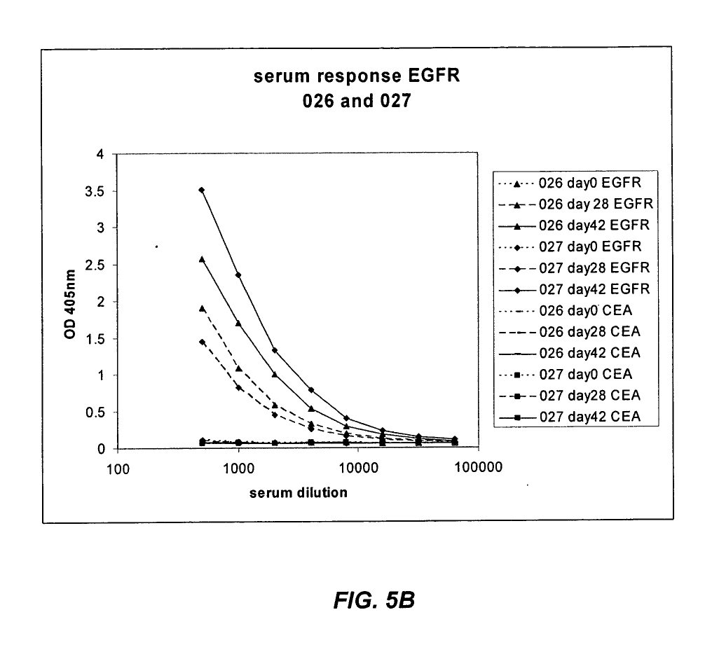

Finally, the serum response in all immunized animals was verified on solid-phase coated purified EGFR. Purified EGFR (Sigma) and the carcino embryonic antigen (CEA, Scripps), both at 1 micrograms/ml, were immobilized overnight at 4° C. in a 96-well Maxisorp plate (Nunc). Wells were blocked for 2 hours at room temperature (RT) with a 1% casein in PBS solution and washed with PBS. After addition of serum dilutions, specifically bound immunoglobulins were detected using a rabbit anti-llama antiserum. Applying a goat anti-rabbit alkaline phosphatase conjugate (Sigma) and using 2 mg of p-Nitrophenyl phosphate (Sigma) substrate per ml of ELISA buffer (1.1M diethanolamine; 1 mM MgSO₄; pH9.8), the calorimetric reaction was spectrophotometrically quantified as OD at 405 nm. A significant humoral immune response against EGFR was induced in llama 024, 025 (FIG. 5A), 026 and 027 (FIG. 5B).

### Example 3

### Obtaining B-Lymphocytes

When an appropriate immune response was induced in the llama, four days after the last antigen injection, a 150 ml blood sample was collected and peripheral blood mononuclear cells (PBMC) were purified by a density gradient centrifugation on Ficoll-Paque™ PLUS (Amersham Biosciences). Typically, a blood sample of 150 ml resulted in the isolation of approximately 10⁸ PBMC. As an alternative source of antibody-producing cells, (biopsies of) lymph node, spleen or bone marrow were collected. Co-purified red blood cells were subsequently lysed by resuspending the Ficoll-purified leucocytes in 20 ml lysis buffer (8.29 g/l NH₄Cl, 1.09 g/l KHCO₃ and 37 mg/l EDTA) at RT, immediately followed by a 200 g centrifugation step for 10 minutes at RT. After erythrocyte lysis, monocytes were depleted by resuspending the remaining cells in 70 ml RPMI (Invitrogen) supplemented with 10% foetal calf serum (FCS), Glutamax™, Hepes (25 mM), penicillin-streptomycin (Invitrogen) and 0.38% sodium citrate. After incubation during 2 hours at 37° C., 5% CO₂ in a T150 tissue culture flask, the supernatant fraction containing the B-lymphocytes was recovered and cells were counted. Typically between 25 and 50% of cells were lost due to this procedure.

### Example 4

### Obtaining EGFR-Specific B-Lymphocytes

EGFR-specific B-lymphocytes were obtained by panning B-cells of Example 3 against EGFR, or with purified receptor or derived from extracellular domains. Prior to B-cell panning, six-well culture plates (Costar) were incubated overnight at 4° C. with 2 ml of 5 micrograms/ml membrane derived vesicles of A431 cells as the enriched EGF receptor fraction. Between 2.5 and 5×10⁷ monocyte depleted cells were resuspended in culture medium supplemented with sodium citrate. Approximately 10⁷ cells were applied to the 6 antigen coated wells and incubated for 2 hours at 37° C., 5% CO₂. Unbound cells were removed by 6 washes with PBS. Subsequently, antigen bound B-cells were eluted by competition for 1 hour at RT with an excess of molecules that compete for the ligand binding site or overlapping epitope(s). The molecules that were used for this epitope specific elution were the EGFR ligands EGF and TGF-alpha, mouse monoclonal antibody (mAb) 2e9 or EGFR antagonistic antibodies 225 and 528 (Sato J D, Kawamoto T, Le A D, Mendelsohn J, Polikoff J, Sato G H 1983. Biological effects in vitro of monoclonal antibodies to human epidermal growth factor receptors. Mol. Biol. Med. 1:511-529). mAb 2e9 is able to internalize the cell via the EGFR receptor and does not activate the receptor (Defize L H, Moolenaar W H, van der Saag P T; de Laat S W 1986. Dissociation of cellular responses to epidermal growth factor using anti-receptor monoclonal antibodies. EMBO J. 5:1187-1192). EGFR antagonistic mAbs 1225 and 528 are able to block the ligand binding site on the EGFR resulting in a decreased-receptor activity. Typically between 10⁴ and 10⁵ cells were recovered after B-cell panning.

Alternative to solid phase immobilization of the antigen, the antigen can be directly (covalently) or indirectly linked to magnetic beads (Dynabeads, Dynal or MACS, Miltenyi Biotech) and used in subsequent panning. When magnetic beads are used for cell selection, cells and an excess of antigen-coated beads are incubated for 10 minutes at 4° C. Optionally, unbound beads may be removed from the mixture by washing the cells with medium. Next, the vessel containing the mixture of bead-labelled cells, unlabelled cells and unbound beads are brought into close contact with a powerful magnet. This magnet system may be stationary or dynamic, such as one where the mixture is allowed to flow through a column filled with inert material to slow down fluid flow and thus prolong the contact with the magnet. Magnetic bead labelled cells will be retained by the magnet, whereas unlabelled cells can be removed by pipetting the fluid phase away from the magnet-immobilized beads in the stationary systems, or by allowing unlabelled cells to flow through the column and/or be flushed from the column by adding excess medium at the top of the column in the dynamic systems. Bead-labelled cells can be retrieved by removing the vessel or column containing the magnetic beads from the magnet and resuspending the bead-labelled cells or flushing the column using medium.

### Example 5

### Preparation of RNA

Eluted B-cells were collected, pelleted and resuspended in 1 ml of TRIzol reagent (Invitrogen), followed by an extraction of total RNA according to the manufacturer's protocol. The presence of cell-bound magnetic beads does not interfere with lysis and subsequent purification steps, but may be removed by holding the vessel containing the TRIzol homogenate to a magnet and pipetting the bead-free fluid from the magnet-retained magnetic bead pellet into a new vessel.

### Example 6

### Trial Extract of mRNA and cDNA Synthesis Using a Small Number of Cells

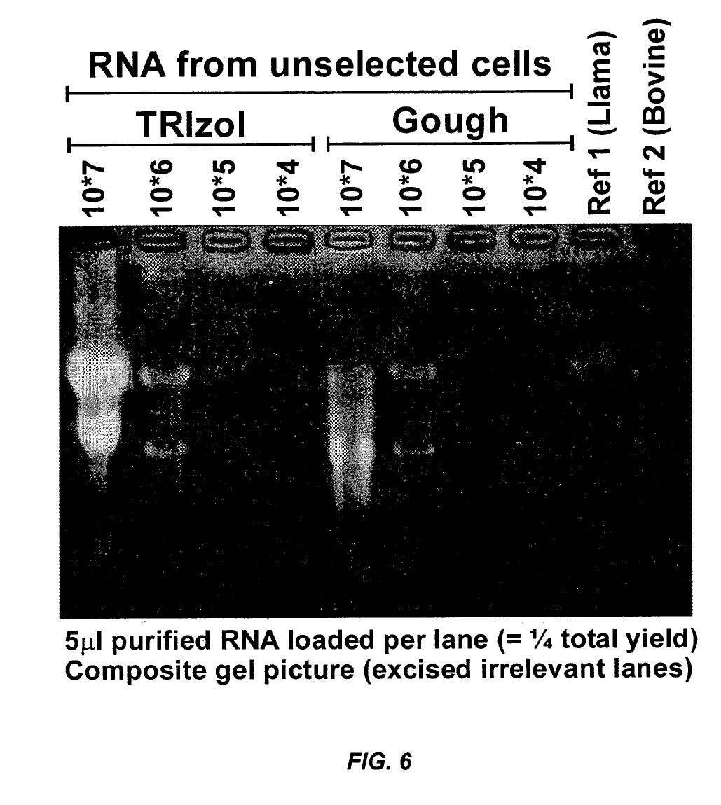

To determine whether RNA could be reliably extracted from small numbers of cells, trial experiments were performed in which RNA was prepare using two methods—TRIzol reagent (Invitrogen) and the Gough method (Gough, N. Mex., Anal Biochem, 1988 173(1); 93-5). Starting with identical pellets of either 10⁷, 10⁶, 10⁵, or 10⁴ LS174T cells, RNA was extracted using both methods in parallel. It was found using the TRIzol method on as few as 10⁴ cells yields sufficient RNA to reliably determine concentrations using a spectrophotometer (FIG. 6; Table 10), whereas the Gough method yielded far less material.

TABLE 10
Isolation of RNA from small cell numbers

| Number of cells | TRIzol RNA yield (ng) | Gough RNA yield (ng) |
|---|---|---|
| 10⁷ | 403 | 147 |
| 10⁶ | 150 | 22 |
| 10⁵ | 3.6 | — |
| 10⁴ | 0.5 | — |

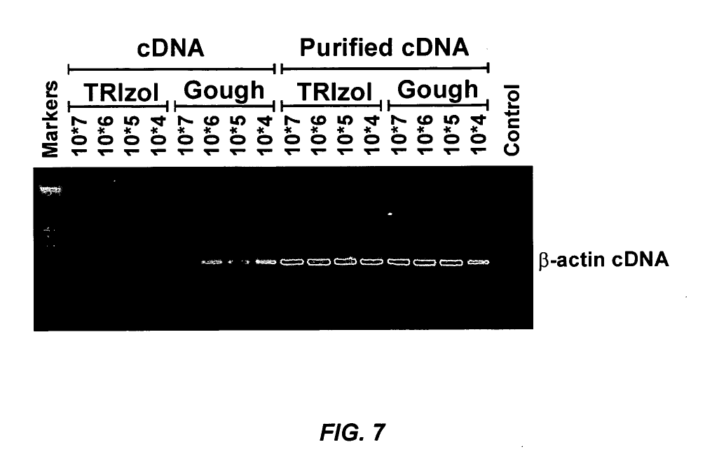

Furthermore, a sufficiently large quantity of beta-actin PCR template grade cDNA could be synthesized from very low amounts of said RNA (FIG. 7).

### Example 7

### Synthesis of cDNA

The complete sample of total RNA was used for oligo-dT or random primed cDNA synthesis using the SuperScript™ III First-Strand Synthesis System for RT-PCR (Invitrogen) according to the manufacturers' recommendations. Next, cDNA was treated with RNAse H to deplete for residual RNA prior to purification with the QIAquick® PCR Purification Kit (Qiagen).

### Example 8

### Nanobody Amplification

Purified cDNA was used as template to amplify the collection of gene segments coding for the variable domain of the heavy-chain antibodies (V_{HH}) originating from the pool of antigen enriched B-cells. The amplification procedure using the combination of a single immunoglobulin specific primer and an oligo-dT primer (described in patent application WO 03/054016), introduced a SfiI restriction site at the 5′ end of framework 1 (FR1). This resulted in a fragment of approximately 1.6 kb (representing the conventional IgGs) and a fragment of 1.3 kb (heavy-chain IgGs) which can be separated by 1.5% agarose gel electrophoresis.

### Example 9

### Cloning Into a Non-Expression Vector

A BstEII restriction site naturally occurs in FR4, therefore, the collection of PCR amplified V_{HH}s was ligated as SfiI-BstEII fragments in the non-expression ‘entry’ vector pAX056a. This ‘entry’ vector is derived from vector pENTR2B of the Gateway® system (Invitrogen) by introducing a multiple cloning site that allows SfiI-BstEII cloning without disrupting the flanking genetic elements required for site-specific recombination. Following this strategy, we obtained a non-expression collection of V_{HH}s resulting from a pool of antigen enriched B-cells. Random colony picking from this non-expression collection allowed us to screen by individual PCRs for presence of an insert with a size of approximately 350-400 bp. Such clones have been safely stored for at least 24 months.

### Example 10

### Cloning Into an Expression Vector

The Gateway® System allows rapid and highly efficient swapping of gene segments (flanked with specific recombination sequences combined with a positive selection marker) to multiple vector systems, allowing convenient subcloning. Therefore, plasmid was prepared from individual Nanobody constructs in pAX056a and used as input material for a series of parallel recombination reactions between the individual ‘entry’ clones and ‘destination’ vector pAX056b using the Gateway® LR Clonase Enzyme Mix (Invitrogen) according to the manufacturer's protocol. This recombination reaction was followed by heat shock mediated transformation of the resulting V_{HH} in pAX56b constructs to E. coli WK6. pAX056b allows the expression of the V_{HH} as a His6- and c-Myc-tagged fusion protein in the periplasmic space of E. coli. Due to leaky expression, supernatant of each culture can be used to screen for antigen binding in ELISA. Following this procedure, it was shown in a cell ELISA comparing signals on Her14 versus 3T3 (Example 2) that approximately 30% of individual clones expressed EGFR specific V_{HH}s.

This method allows efficient swapping of V_{HH} between the non-expression vector and a Gateway® compatible vector of choice optimized for functional screening. An example of such destination vector is an expression vector that allows efficient recloning of the Nanobody-as multivalent constructs.

In this system the use of an entry vector with multiple flanking specific recombination sites permits in a single recombination step the construction of a bivalent genetically fused Nanobody, thus allowing the generation of large numbers of clones in high-throughput mode. With this method highly potent antagonistic EGFR antibodies were identified in bivalent format. In addition, fusion of anti-EGFR Nanobodies to anti-albumin Nanobodies were generated, thereby permitting the identification of Nanobodies in which the albumin binding did not affect the binding to the EGF receptor.

### Example 11

### Trial Selection and cDNA Preparation of Collagen-Binding Splenocytes in Mice

Two mice were immunized with collagen. After the final booster immunization, spleens removed therefrom and splenocytes prepared. Around 1.4×10⁸ splenocytes were obtained. After monocyte depletion, the number of remaining cells was 1.1×10⁸.

Antigen Selection

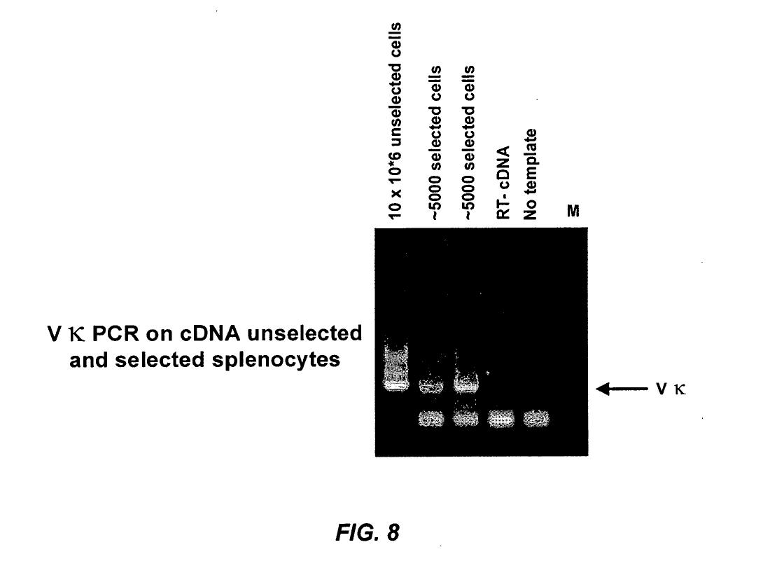

Around 10⁸ monocyte-depleted PBMCs were used for antigen selection. Plates were coated with collagen, incubated with monocyte-depleted splenocytes and thoroughly washed to remove unbound cells. Approximately 5000 cells remained attached to the plate. RNA was isolated from these selected cells and cDNA prepared therefrom. V kappa PCR on the cDNA indicated successful amplification of immunoglobulin variable gene segments after antigen selection of conventional 4-chain antibody-forming B-cells (FIG. 8).

### Example 12

### Selection of Human C1q Protein Using Biotinylated C1q in Combination With Anti-Biotin MACS Beads

Llama 40 was immunised with human C1q protein. After the final booster immunization, PBMCs were prepared and frozen at −80° C. Approximately 3×10⁷ viable PBMCs were obtained after thawing and washing. Approximately 1.3×10⁷ cells remained after monocyte depletion.

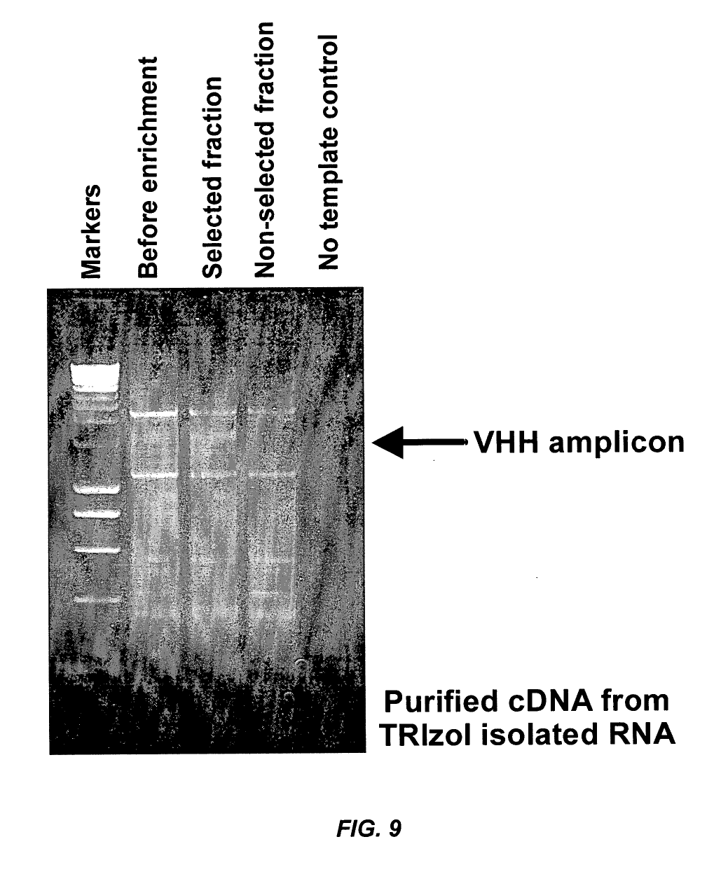

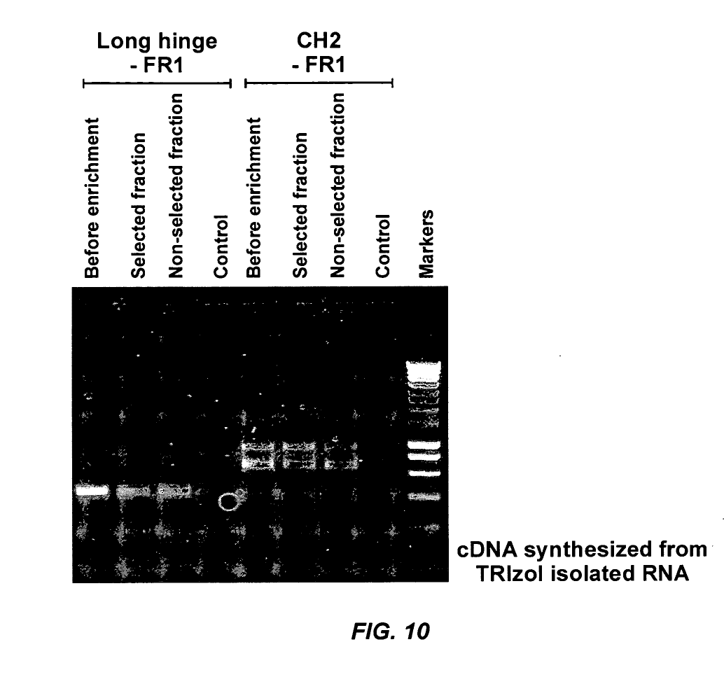

Around 1.2×10⁷ monocyte-depleted PBMCs were used for antigen selection using MACS (Miltenyi Biotec). MACS anti-biotin pre-coated beads were incubated with biotinylated C1q, washed to remove unbound biotinylated C1q and incubated with monocyte-depleted PBMC. After washing the cells, bead-labelled and unlabelled cells were separated using a LD model column firmly lodged in a QuadroMACS magnet. After separation, around 5×10⁵ (4%) monocyte depleted PBMCs which selectively bind human C1q protein were obtained. RNA and cDNA was prepared therefrom (FIGS. 9 and 10).

### Example 13

### Selection of HuCD28/Fc-Gamma Fusion Protein Using Selection Based on MACS Beads, Dynal Beads or Plates

Llama 045 was immunised with human CD28/human IgG1 fusion protein. PBMCs were prepared after the final immunization and about 10⁸ cells were obtained. After monocyte depletion, 7.6×10⁷ cells remained.

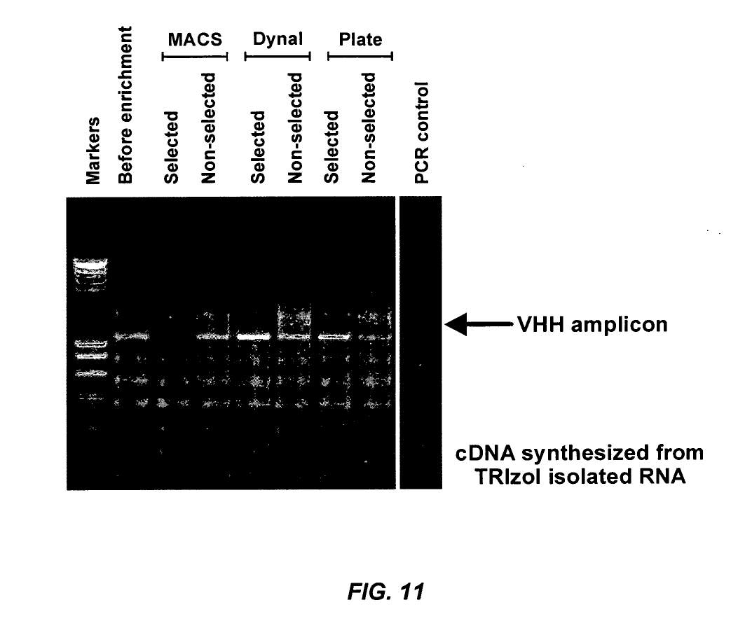

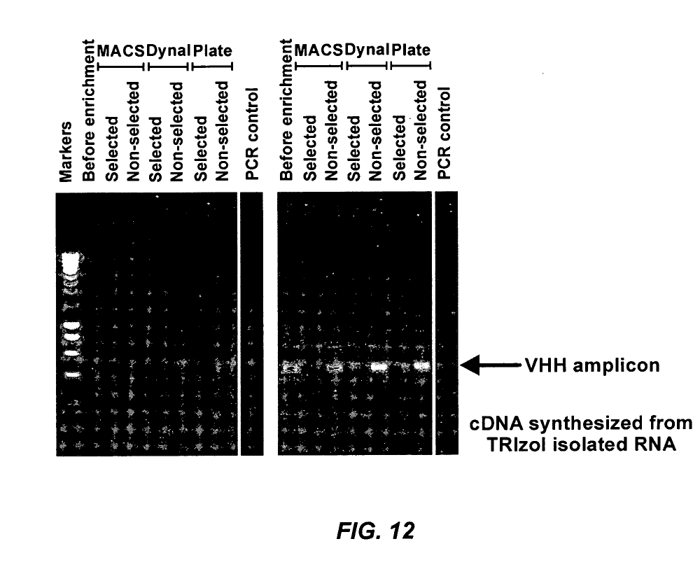

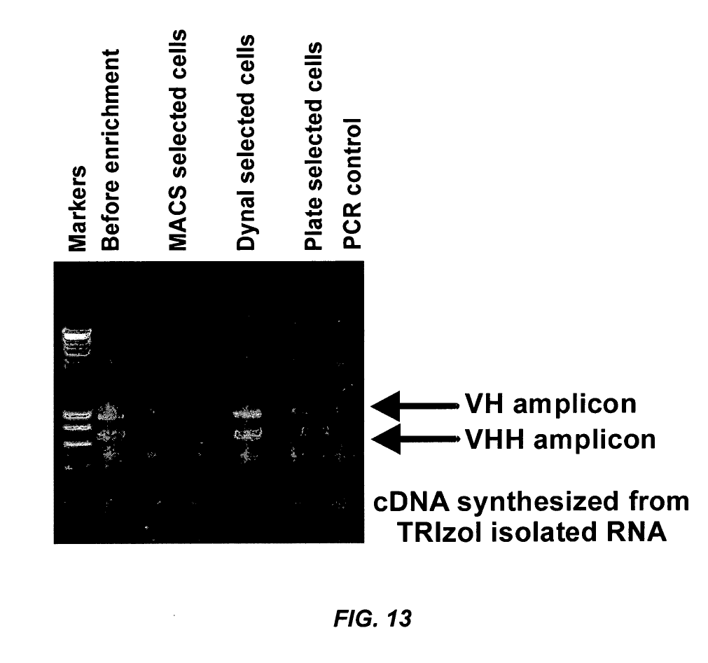

Around 2.2×10⁷ monocyte-depleted PBMCs were used for antigen selection using MACS beads (Miltenyl Biotec). Anti-human IgG1 antibody pre-coated MACS beads were coated with CD28/IgG1 fusion protein by simply incubating beads in a CD28/IgG1 fusion protein solution. Next, beads were washed and brought into close contact with cells in the presence of heat-inactivated llama serum from an un-immunized animal. Incubation with this serum effectively blocks the possible interaction of human IgG1 Fc with Fc receptor on llama lymphocytes, without masking any CD28 epitopes with soluble llama antibody as might occur when using immune llama serum. Heat-inactivation of the serum prevents any llama serum complement-driven cell lysis. After a 10′ incubation period, excess beads were removed by a simple washing step and bead-labelled cells were separated from non-labelled cells by passing the labelled, washed cells through a LD column held in a QuadroMACS magnet. After separation, around 1.1×10⁴ (0.5 per milliliter) monocyte-depleted PBMCs binding HuCD28/Fc-gamma fusion protein were obtained from the column after removing it from the magnet. Total RNA and cDNA was prepared therefrom (FIGS. 11, 12, and 13).

Around 2.2×10⁷ monocyte-depleted PBMCs were used for antigen selection using Dynal M450/tosyl antigen conjugated beads. Tosyl surface-activated Dynabeads were coated with HuCD28/IgG1 fusion protein, blocked with milk casein to block any further covalent protein binding, washed repeatedly using a magnet to remove free protein and brought into close contact with monocyte-depleted PBMC. After a 10 min incubation period at 4° C., bead-bound cells were separated from unlabelled using a Dynal magnet. After separation and repeated washing of the beads using medium a small aliquot of selected bead-cell complexes were removed for counting using trypan blue and a haemocytometer. Around 3×10⁵ (1.5%) monocyte depleted PBMCs selectively binding the human CD28 portion of the HuCD28/Fc-gamma fusion protein were obtained RNA and cDNA was prepared therefrom (FIGS. 11, 12, and 13) by lysis of the cell-bead complexes in TRIzol.

In parallel, around 2.2×10⁷ monocyte-depleted PBMCs were used for antigen selection on antigen-coated plate. Plates were coated with HuCD28/IgG1 fusion protein and plate-bound cells were selected in the presence of heat-inactivated llama serum, as described above. Around 5×10⁴ (2.5 per milliliter) HuCD28-binding cells were selected. RNA and cDNA was prepared therefrom by TRIzol lysis in the wells (FIGS. 11, 12, and 13).

The percentage antigen-binding Nanobody clones from CD28/Fc-gamma selected and unselected llama B-cell populations is presented in Table 11.

TABLE 11
Percentage antigen-binding Nanobody clones from CD28/Fc-gamma
selected and unselected llama B-cell populations

| | | | Insert- | |
|---|---|---|---|---|
| | | | corrected | |
| | | | antigen- | Enrich- |
| | Antigen | Insert | binding | ment |
| mRNA origin | binding | containing | clones | factor |
| Unselected | 3/96 (3%) | 48/48 (100%) | 3% | — |
| MACS selected | <1/48 (<2%) | 18/24 (75%) | <3% | ≦1 |
| Dynal selected | 14/48 (29%) | 20/24 (83%) | 34% | 11 |
| Plate selected | 7/48 (15%) | 24/24 (100%) | 15% | 5 |

The percentage antigen-binding Nanobody clones from Dynal selected B-cells, cloned into non-expression vector and individually recloned into expression vector via Gateway® is presented in Table 12.

TABLE 12
Percentage antigen-binding Nanobody clones from Dynal selected B-cells,
cloned into non-expression vector and individually recloned
into expression vector via Gateway ®.

| | | | Insert-corrected |
|---|---|---|---|
| | Antigen | Insert | antigen-binding |
| Method | binding | containing | clones |
| Cloned in expression | 14/48 (29%) | 20/24 (83%) | 34% |
| vector | | | |
| Cloned in non-expression | 7/24 (29%) | 24/24 (100%*) | 29% |
| vector, recloned into | | | |
| expression vector | | | |

*inferred from double selection on lack of suicide gene and presence of antibiotic resistance in Gateway ® acceptor plasmid.

### Example 14

### Selection of Human Integrin Alpha-v-Beta-5 Protein Using Selection Based on MACS Beads, Dynal Beads or Plates

Llama 043 was immunised with human integrin alpha-v-beta-5 protein, and PBMCs were prepared after the final immunization. Around 10⁷-10⁹ PBMCs were obtained and used for monocyte depletion. Approximately 10⁸ non-adherent cells were recovered.

The monocyte-depleted cell suspension was then split into four roughly equal aliquots, one of which served as a reference sample and three of which were used in three parallel antigen selections.

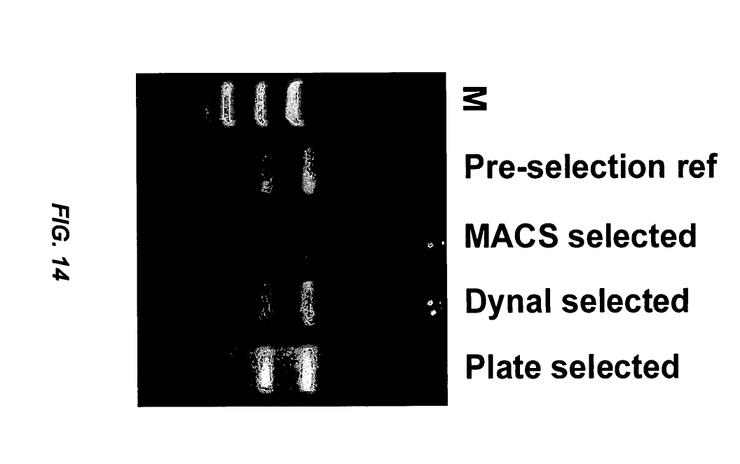

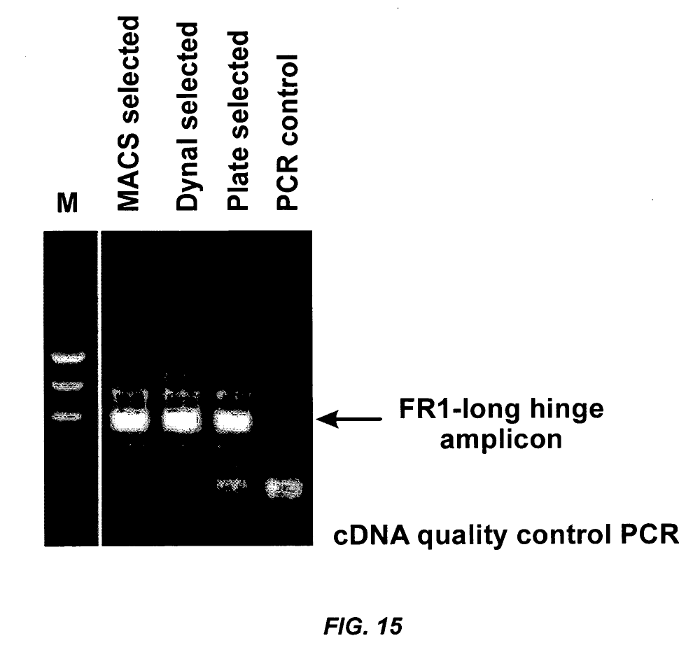

Around 2×10⁷ monocyte-depleted PBMCs were used for antigen selection using MACS (Miltenyi Biotec). Anti-biotin precoated MACS beads were coated with biotinylated alpha v-beta-5 integrin by incubating the beads in the biotinylated protein solution overnight at 4° C. Excess alpha v-beta-5 integrin was removed by washing the beads prior to mixing the monocyte-depleted cell suspension with the antigen-coated beads and co-incubating these for 15′ at 4° C. Next, cells were washed and bead-binding cells selected using LD column placed in a QuadroMACS magnet according to the manufacturer's instruction (Miltenyi Biotec). After separation, around 2.56×10⁵ (1%) monocyte-depleted PBMCs which selectively bind human integrin alpha-v-beta-5 protein were obtained. RNA and cDNA was prepared therefrom (FIGS. 14 and 15) by lysing the bead/cell complexes in TRIzol reagent.

In parallel, another aliquot of around 2×10⁷ monocyte-depleted PBMCs were used for antigen selection using Dynal M450/tosyl antigen conjugated beads. Pre-activated Dynal beads were covalently coated with human integrin alpha-v-beta-5 protein and free remaining binding sites blocked using the protocol provided by the manufacturer. Cells were mixed with the coated beads, incubated at 4° C. and subsequently placed in a Dynal MPC-S magnet. Bead binding and non-binding cells were separated by pipetting off the fluid while beads and bound cells remained immobilized on the tube wall by the magnet. The tube-retained bead/cell mass was repeatedly resuspended in new cell culture medium and re-extracted from it using the magnet to remove non- or weakly binding cells. After the final wash, around 1.92×10⁵ (1%) monocyte-depleted PBMCs which selectively bind human integrin alpha-v-beta-5 protein were obtained. RNA and cDNA was prepared therefrom (FIGS. 14 and 15) by lysing the bead/cell complexes in TRIzol reagent.

In parallel, a third aliquot of around 2.2×10⁷ monocyte-depleted PBMCs were used for antigen selection by plate. A Greiner 6-well tissue culture plate was coated overnight at 4° C. with human integrin alpha-v-beta-5 protein solution, after which the solution was removed and wells were washed using fresh cell culture medium. The suspension of monocyte depleted cells was then distributed across all 6 wells and incubated for 1 hour at 37° C. in a CO₂ incubator. Next, all wells were repeatedly and vigourously rinsed using fresh cell culture medium to remove non-binding cells. Around 10⁵ (1%) or less human integrin alpha-v-beta-5 protein-binding cells were obtained as estimated by microscopic inspection of the plate. RNA and cDNA was prepared therefrom (FIGS. 14 and 15) by lysing the plate-bound cells in TRIzol reagent.

The percentage of integrin alphaᵥbeta₅-binding Nanobody clones from antigen-selected and unselected llama B-cell populations is presented in Table 13.

TABLE 13
Percentage integrin alphaᵥbeta₅-binding Nanobody
clones from antigen-selected and unselected llama B-cell populations

| mRNA origin | Antigen binding | Enrichment factor |
|---|---|---|
| Llama 043 | | |
| Unselected | 2/24 (8%) | — |
| MACS selected | 9/24 (37%) | 4.6 |
| Dynal selected | 12/24 (50%) | 6.3 |
| Plate selected | 17/24 (71%) | 8.9 |
| Llama 052 | | |
| Unselected | 4/23 (17%) | — |
| MACS selected | 13/24 (54%) | 3.2 |
| Dynal selected | 8/23 (35%) | 2.1 |
| Plate selected | 4/24 (17%) | 1.0 |

### Example 15

### Evaluation of Functional Nanobody Expression in Vectors

After cloning the gene of an anti-TNF-alpha Nanobody from a non-expression vector into an expression vector using site-specific recombination (See FIG. 2), expression of this Nanobody in the pAX51 and pAX56b vectors was tested in E. coli WK6.

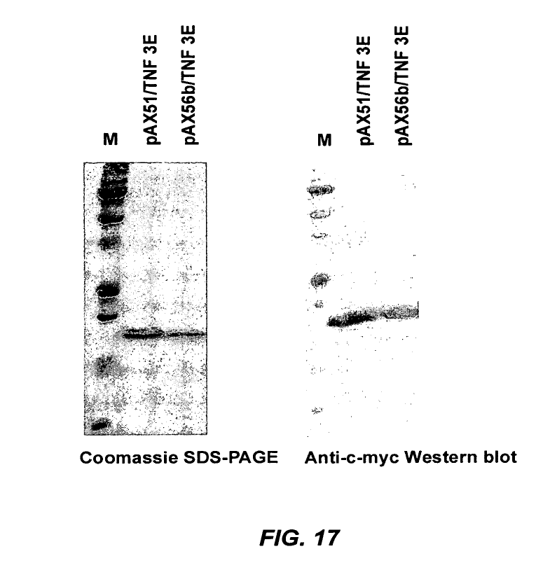

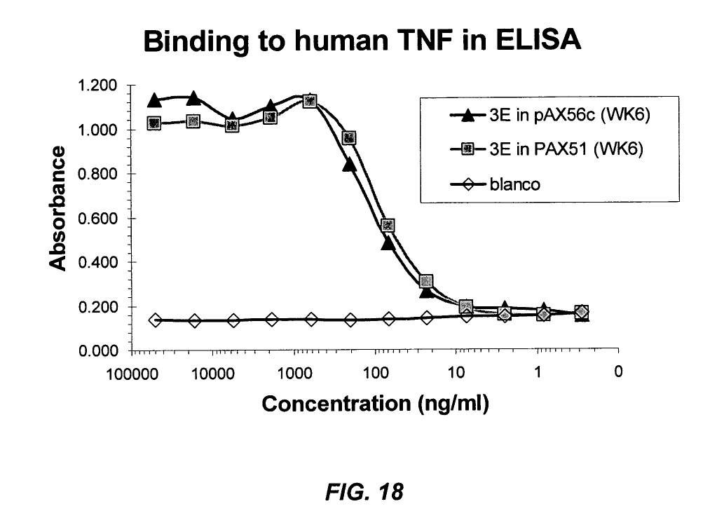

The results indicate good levels of expression and functional TNF-binding for “3E” Nanobody (WO 04/041862) expressed from both pAX51 and pAX56b vectors (FIGS. 17 and 18).

### Example 16

### Depletion of Conventional Antibodies

A defining difference between conventional and heavy chain antibodies is the absence of a paired light chain in the latter. Therefore, B-cells producing heavy-chain antibodies are not thought to have kappa or lambda light chains paired to their surface-bound immunoglobulin either. In Ablynx's flow cytometric experiments, phycoerythrin (PE) labeled polyclonal anti-human kappa- and lambda-chain antisera (Caltag Labs, Inc.) were found to recognize a subpopulation of lymphocytes in Lama glama PBMC samples (data not shown). Therefore, labeling of B-cells displaying conventional (kappa- or lambda-chain paired) immunoglobulins on their cell membrane with such reagents, followed by MACS immunomagnetic depletion of PE tagged cells (Miltenyi Biotech anti-PE microbeads), will deplete the conventional repertoire from a given PBMC sample irrespective of antigen reactivity. By combining this step with antigen-binding enrichment of antigen-reactive B-cells, carry-over of conventional repertoire B-cells into the antigen-reactive enriched cell fraction can be heavily reduced, if not eliminated outright.

### Example 17

### Convenient Screening for Avidity Effects in Bivalent Formatted Monospecific Nanobodies

Avidity effects can contribute significantly to the therapeutic efficacy of multivalent protein binding molecules. However, epitope fine specificity differences and the resulting steric hindrance between two binding sites on different bivalently formatted monovalent Nanobodies directed against the same protein may render some Nanobodies unsuitable for use in bivalent formats. A convenient and fast cloning method for screening many monovalent binders for potential avidity effects early in the development process is therefore desirable. A description of such a method follows.

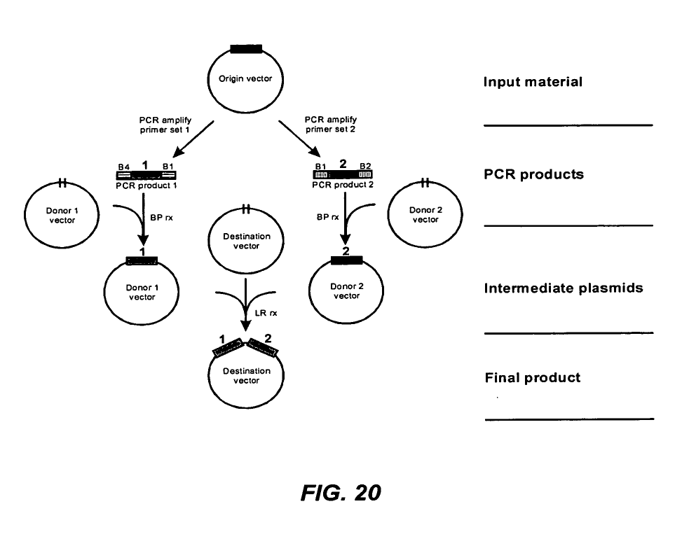

A large set of monovalent Nanobodies in a cloning “origin” vector is amplified in two parallel sets of PCR reactions using two universal primer sets; coded Primer set 1 and Primer set 2 (FIG. 20). Primer set 1 contains a 5′ primer encoding the B4 sequence in addition to a FR1 consensus sequence, as well as a 3′ primer encoding a B1 sequences in addition to a FR4 consensus sequence. PCR amplification therefore results in a single PCR product “1” containing the monovalent Nanobody sequence, flanked by the B4 and B1 recombinase recognition sites. Primer set 2 contains similar 5′ and 3′ primers encoding the B1 and B2 sites respectively, and yields a single PCR product “2”. By dispensing small amounts of template Nanobody encoding plasmids from a “master” microtiter plate into two replica microtiter PCR plates containing a PCR mix with primer set 1 and primer set 2 respectively, two sets of linear modified sequence Nanobody DNA are generated from 96 or 384 clones in parallel.

Both sets of amplicons are integrated into two different intermediate “donor” vectors 1 and 2 using the Invitrogen™ Gateway® BP recombination reaction by adding the PCR products “1” and “2” to microtiter plate wells containing plasmid 1 and 2 respectively, and recombinase mix. These two vectors contain recombinase recognition sites matching the B4/B1 and B1/B2 sites respectively, added to the Nanobody clone sequence by the PCR primers described above. After the recombination reactions have taken place, the resulting recombinant plasmids are transfected by heat shock into competent E. Coli cells pre-aliquotted into another microtiter plate. The cells are grown in deep-well plates under antibiotic selection pressure and plasmid is purified from all wells.

By mixing both resulting parallel plasmids containing the same Nanobody sequence with a final “destination” expression plasmid in a Gateway® LR recombination reaction mix, both versions of the same Nanobody gene are linked in a pre-determined order into a single gene encoding a fusion protein of both on an expression plasmid, linked by a Gateways® recombination sequence encoded linker and carrying an affinity protein purification tag. The resulting plasmid is then transfected into heat-competent E. coli cells, which are grown and selected in deep-well microtiter plates, all as described above.

Aliquots of all parallel mini-cultures are harvested for purification of the fusion protein, taking advantage of the affinity purification tag. The purified fusion proteins are analyzed for evidence of avidity effects in various antigen binding assays.

Automated liquid handling systems, thermal cyclers and reagent kits/consumables for handling all microtiter plate replication, PCR, PCR product/plasmid/protein purification and transfection procedures are readily available. Thus, the combination of the availability of simple mix-and-incubate Gateway® recombination reactions, laboratory robotics and the formatting flexibility of the Nanobody platform makes screening for avidity in multivalent formats easier than is the case for conventional antibody or restriction enzyme cloning technologies.

### Example 18

### Convenient Screening for Lack of Deleterious Loss-of-Function Effects in Bispecific Formatted Multivalent Nanobodies

Similar in concept to the above example, it is highly advantageous to screen for lack of mutual antigen binding steric hindrance problems in bispecific fusion proteins early in the development cycle. A description of such a method follows.

As illustrated in the previous example, 5′ and 3′ Gateway® BP recombinase recognition sites are added to a given Nanobody clone sequence located in an “origin” plasmid by using various PCR primer sets. These PCR products are cloned into intermediate “donor” plasmids using the BP recombination reaction. Recombination of two resulting donor plasmids with a “destination” vector in a single homogeneous LR multisite recombination reaction then yields the final fusion protein gene of the input Nanobodies in a predefined order in an expression vector.

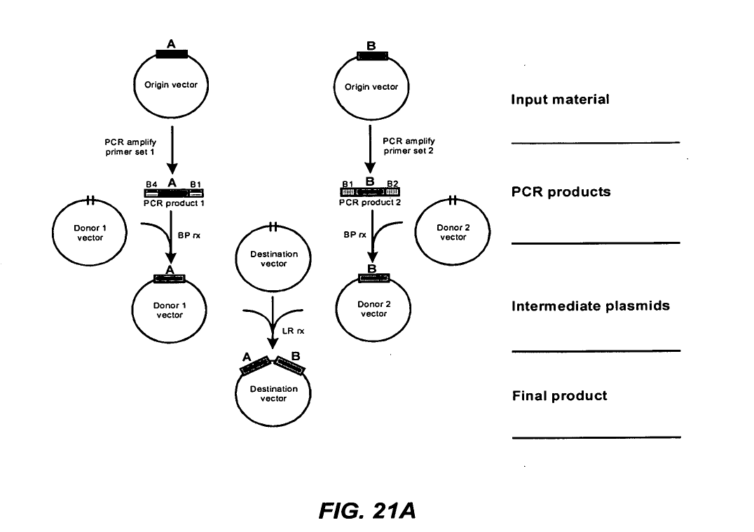

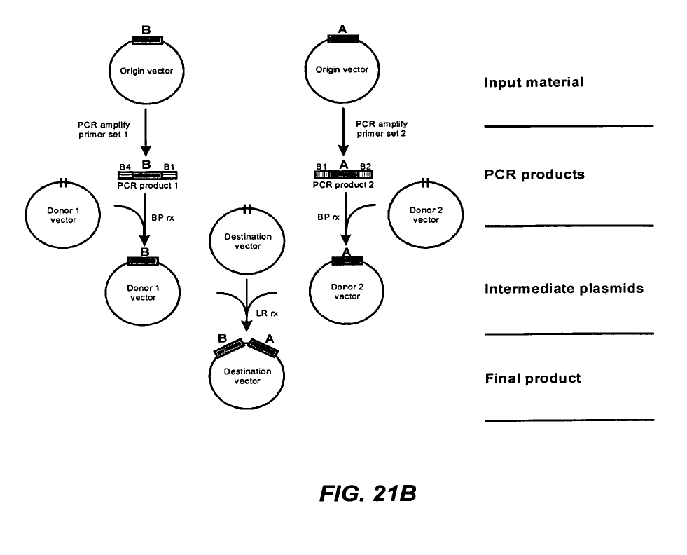

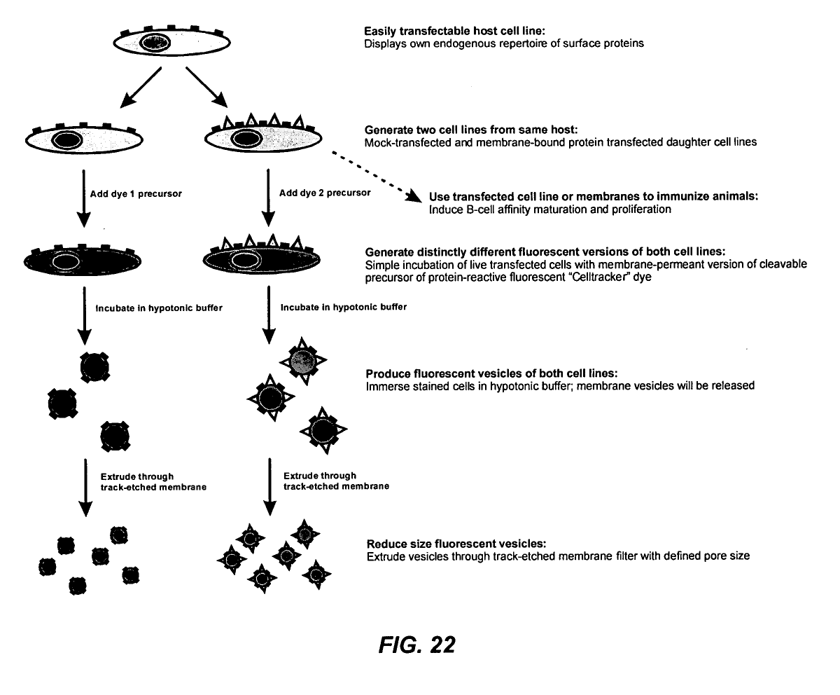

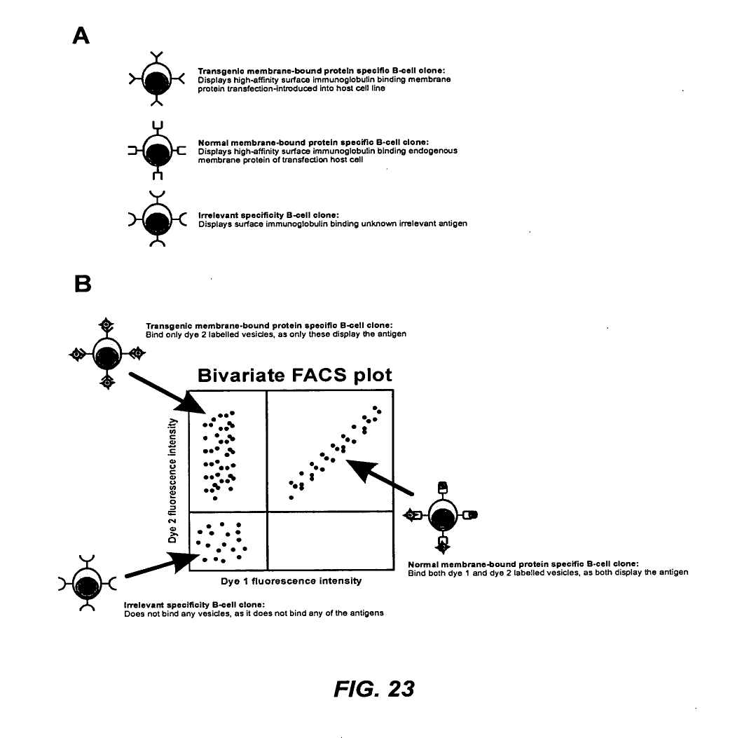

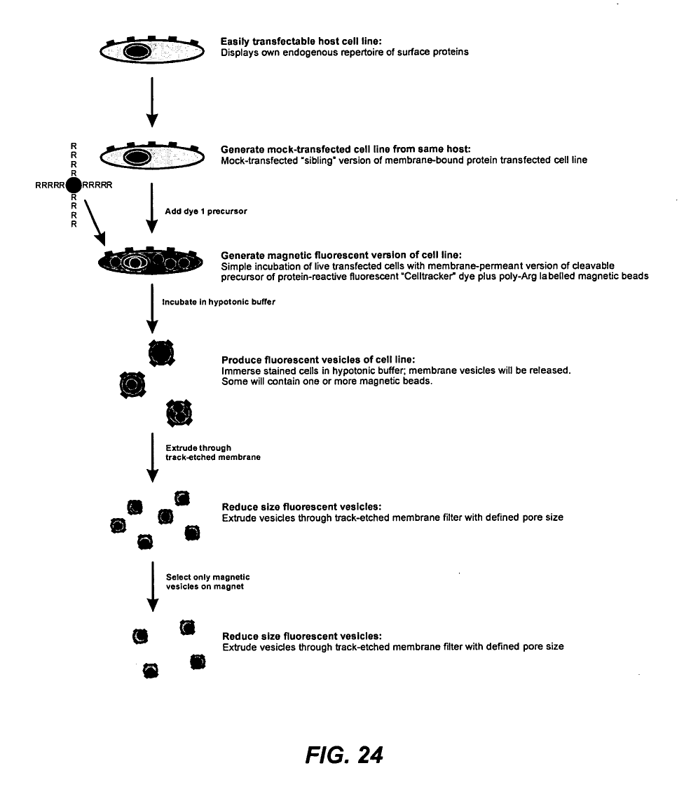

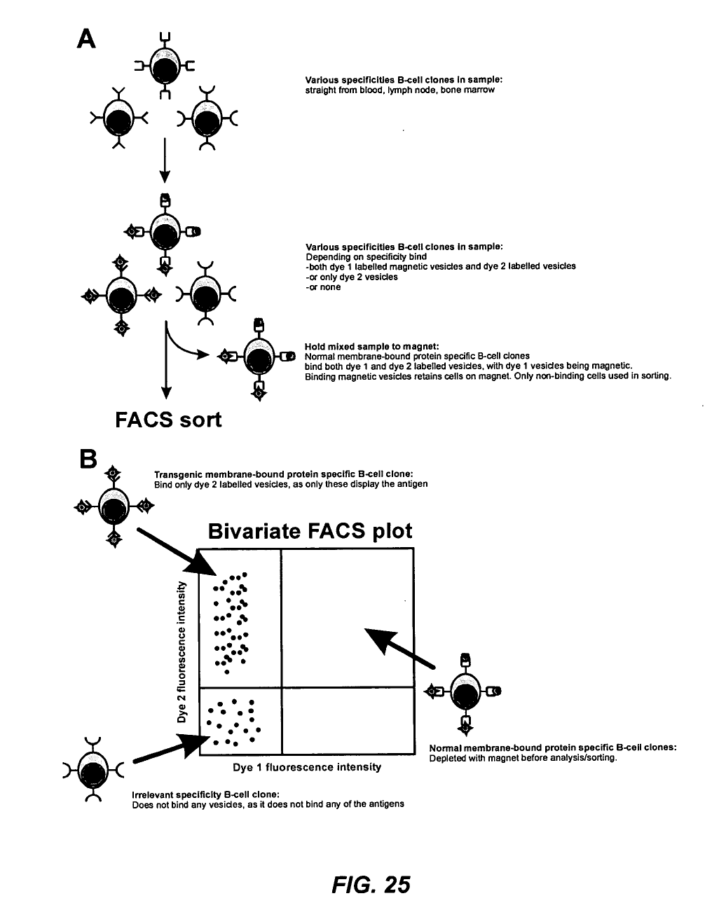

Therefore, by using two different Nanobody clone containing vectors as template in the PCR primer set 1 reaction and the PCR primer set 2 reaction, rather than the same, donor 1 and donor 2 intermediate plasmids are generated in parallel containing two different Nanobodies (FIG. 21A). By LR recombining a Nanobody of a given specificity A in the donor 1 vector with a Nanobody of a different specificity B in the donor 2 vector, the fusion protein encoded on the resulting recombinant vector will be bispecific rather than monospecific bivalent. The fusion proteins are produced and purified using destination vector-encoded affinity purification tags as described above. The resulting proteins are screened for bifunctionality in vanous assays.

An additional attractive feature of this technology is that one can use the identical input material and reagents to screen both possible orientations of the fusion protein, that is, check bispecificity of the fusion protein with specificity A Nanobody N-terminal to specificity B Nanobody (FIG. 21A), or with specificity B Nanobody N-terminal to specificity A Nanobody (FIG. 21B). To accomplish this, first the input plasmid of Nanobody specificity A is amplified with primer set 1 and recombined into intermediate donor vector 1. Conversely, Nanobody specificity B is amplified with primer set 2 and recombined into donor plasmid 2; LR recombination from these intermediates results in a Nanobody A—Nanobody B bifunctional fusion protein. By amplifying specificity B Nanobody with primer set 1 and recombining the product into donor plasmid 1, likewise for specificity A with primer set 2 and donor plasmid 2, the LR recombination product will encode Nanobody B—Nanobody A fusion protein.

### Example 19

Immune and Non-Immune Llama B-Cell Samples

To prepare control cells for flow cytometric cell sorter setup, one small batch of frozen PBMC from an unimmunised animal (llama #58) was thawed and washed by adding 5 ml DMEM/F12 medium containing 10% bovine calf serum (DMEM/F12+10% BCS) and subsequent centrifugation for 15′ at 200 g. The cell pellet was resuspended in 6.25 ml DMEM/F12+10% BCS and depleted for monocytes by their selective adherence to the surface of a T25 culture flask during a 2 hr incubation at 37° C. 0.55×10⁶ viable suspension cells were retrieved from the flask.

To prepare monocyte depleted PBMC from a llama immunised with human TNF alpha (llama #54), a vial containing 90×10⁶ PBMC isolated from a fresh blood sample and frozen in 90% foetal calf serum/10% DMSO was thawed and the cells washed essentially as described above but using 30 ml DMEM/F12+10% BCS medium. Using a haemocytometer and trypan blue exclusion, a recovery of 71.5×10⁶ cells was determined, 68×10⁶ of which were viable (3.5×10⁶ or 4.9% of total were non-viable cells). These were then depleted for monocytes by diluting the stock using fresh cell culture medium to 37.5 ml and incubation for 2 hr at 37° C. in a T150 culture flask (1.81×10⁶ cell/ml cell density). 52.5×10⁶ viable non-adhering cells were recovered from the flask (4.2×10⁶ or 7.4% of total were non-viable cells).

Human TNF Binding B-Cell Selection

TNF panning was undertaken to enrich the cell suspension for cells expressing IgG capable of binding TNF. The llama #54 monocyte depleted cell sample was split into 3 aliquots: 2 aliquots of 20×10⁶ cells (for plate and MACS selections on TNF, respectively) and 1 aliquot of 12×10⁶ cells (plate selection for total B-cells, as a control experiment).

For plate selection on TNF, all wells of a 6-well tissue culture plate were coated overnight at 4° C. with 4 ml/well of 10 microgram/ml neutravidin (Sigma) in PBS. Wells were washed 3× with 4 ml PBS each and incubated for 1 hr at 37° C. with a 1:500 dilution in PBS of a 0.8 mg/ml stock solution of biotinylated recombinant human TNF alpha. Wells were again washed 3× with PBS prior to adding the 20×10⁶ cells monocyte depleted PBMC suspension to the wells. The cells were then incubated for 1.5 hr at 37° C. in a 5% CO₂ incubator. After incubation, all wells were washed 6× with 4 ml/well of fresh PBS to remove cells not binding plate-bound TNF. Cells remaining plate-bound after these washes were eluted from the plate adding 800 microliter/well of 5 mM EDTA in PBS and incubating for 5 min in this buffer, after which 1 ml DMEM/F12+10% BCS was added to all wells and remaining non-eluted cells were harvested by applying a cell scraper to the plate. The number of eluted cells was too low to accurately count or determine viability (yield less than 10⁴ cells).

For MACS selection on TNF, a 20×10⁶ monocyte depleted PBMC stock aliquot was resuspended in 4° C. freshly degassed PBS/2% FCS at a density of 100×10⁶ cells/ml. Biotinylated TNF was added to the cells ( 1/200 final dilution), mixed gently and incubated for 15 min at 4° C. Cells were then washed twice using 10 ml of 4° C. degassed PBS/2% FCS and resuspended in 80 microliter of 4° C. degassed PBS/2% FCS, after which 20 microliter of anti-biotin MACS beads stock was added. Cells and beads were co-incubated for 15 min at 4° C. The cells were then washed again using 10 ml of 4° C. degassed PBS/2% FCS, resuspended in 500 microliter of 40° C. degassed PBS/2% FCS and applied to a MACS prefilter placed on top of a prewetted LS column placed in a QuadroMACS magnet (beads, column, magnet all from Miltenyi Biotec). The buffer was allowed to drain from the column. The column was then washed three times by applying 3 ml of 4° C. degassed PBS/2% FCS to the prefilter and always allowing the column to stop dripping between washes. Next, the prefilter was removed from the column and the column removed from the magnet. 5 ml of 4° C. degassed PBS/2% FCS was applied to the top of the column and positively selected cells were collected into a clean new centrifuge tube by applying pressure to the top of the column using the sterile plunger provided with the column 2 ml of fresh buffer was reapplied to the column and the elution procedure was repeated; the resulting eluted cell suspensions were pooled by collected them in the same tube. Column-eluted cells were centrifuged down and the topmost buffer removed until a final volume of 500 microliter was left in tube. The cells were resuspended in this volume of medium. Viable cell yield of the antigen selected fraction was determined to be approximately 5×10⁵, with 2×10⁵ additional dead cells (28% of total yield antigen selection).

For experiment reference purposes, B-cells were isolated non-antigen selectively from an aliquot of the same monocyte depleted cell sample. This was accomplished by panning the 12×10⁶ cells aliquot in 3 wells of a 6-well tissue culture plate previously coated overnight at 4° C. using 4 ml per well of 2 microgram/ml of goat-anti-llama IgG (Bethyl Laboratories) in PBS, using the same washing and elution steps as described for plate selection on TNF, described above. Approximately 7.5×10⁴ viable cells were obtained after elution. The goat-anti-llama IgG polyclonal antiserum from Bethyl was determined previously in both Western blot and ELISA to bind 4-chain type llama IgG (“IgG1”) as well as both types of heavy chain antibody (“IgG2” and “IgG3”), so no selectivity towards either type of B-cell is expected.

Cell Staining and Sorting

Contamination of the antigen (or control) selected B-cell populations with conventional repertoire expressing B-cells was expected, but only heavy chain antibody producing B-cells are relevant for downstream cloning of Nanobodies. Also, a method by which a known, small number of viable cells is deposited in all or some wells of many microtiter plates used for B-cell stimulation was required. Therefore, a flow cytometric cell sorter was used to dispense a known number of cells in all wells of B-cell stimulation microtiter plates while simultaneously rejecting non-viable and/or 4-chain immunoglobulin displaying B-cells from the sort. Dead cells were excluded from the sort based on negative gating for staining with the dead cell specific stain propidium iodide (PI, from Sigma), whereas 4-chain immunoglobulin displaying B-cells were rejected based on negative gating for staining with two llama crossreactive goat-anti-human kappa and goat-anti-lambda light chain specific reagents, both labeled with the same fluorophore phycoerythrin (PE) (goat-anti-human kappa-PE, goat-anti-human lambda-PE both from Caltag).

Cells eluted in all three selection procedures described above were stained for kappa and lambda expression immediately after plate/column elution, by adding 5 microliter stock solution per 150 microliter cell suspension of each of both fluorescent antibodies to the cell suspensions. Stain/cell mixtures were incubated for 30′ at 4° C. in the dark and washed repeatedly to remove excess stain. 1 microliter of PI stock (1 mg/ml in PBS) was added to the washed cells suspension 5 minutes prior to analysis.

In order to properly configure the flow cytometer's light scatter and fluorescence detection channel settings, as well as compute the compensation matrix for PI and PE fluorescences, many more cells are required and lost during the procedure than can be obtained through the selections mentioned above. Therefore, a much larger sample of llama PBMC irrelevant to the sorting procedure was prepared (non-immune llama #58; for procedure, see top of example) and used unstained to optimize forward/side scatter settings. Aliquots of the same population were stained only with the anti-kappa and anti-lambda reagents, only with PI or with both according to the method described above. These none, single- and double-positive populations were then used to set the proper fluorescence detection channel parameters as well as set the appropriate PI/PE compensation matrix. Similarly, cell sorter drop delay settings and sorted droplet deposition into 96-well microtiter plates were optimized using this irrelevant sample.

Once optimal settings for detection and sorting had been defined, the PE/PI double-stained antigen or control selected B-cell samples were analyzed. A positive sorting gate was set on intact lymphocytes based on forward/side scatter profile, with negative gates set for PI and PE positive stainings. Only cells within the positive gate but outside both negative gates were sorted at 3 or 6 cells per well into 96-well tissue culture microtiter plates, all containing 100 microliters of cell culture medium.

Sorted B-Cell Stimulation

For in vitro stimulation of sorted B-cells, 50,000 irradiated EL4-B5 cells (2500 rad, Gamma cell 3000, Elan, MDS Nordion) were dispensed in all wells of a series of 96-well microtiter plates in 200 microliter volumes of DMEM/F12+10% BCS medium containing 1% llama T-cell conditioned supernatant (TSN) per well. The llama T-cell conditioned supernatant was previously prepared by in vitro stimulation of PBMC isolated from several non-immune llamas using 1 microgram/ml of phytohaemagglutinin (PHA, Sigma). The cell-free supernatant from these cultures was depleted for immunoglobulins prior to use in B-cell culture by passing it over both a protein A and a protein G column.

All wells but four in all plates received 3 sorted B-cells per well. B-cell culture plates were incubated at 37° C., 5% CO₂ for a total of 11 days. Medium was refreshed once with new medium containing 1% llama TSN on day 3 of the culture.

B-Cell Culture Conditioned Medium Screening

In order to determine the cloning efficiency of sorted B-cell stimulation, the B-cell culture conditioned medium of all wells was analyzed for production of llama immunoglobulins. To do so, Nunc Maxisorp ELISA plates were coated overnight at 4° C. with 50 μl per well of goat anti-llama IgG (Bethyl) diluted 1:1000 times in PBS, washed the next morning, blocked with 1% casein in PBS for 1H at room temperature and washed again. Next, 50 μl B-cell culture supernatant was harvested from the top of all B-cell culture plate wells without disturbing the cell pellet, diluted with 1:2 using 50 μl of PBS containing 1% BSA and 0.05% Tween-20 and then placed into the wells of the ELISA plates. 1 microliter of serum, taken from the same blood sample of llama #54 which was used to select TNF binding B-cells, was added to two wells per plate corresponding to two of the four wells not having received sorted B-cells in the B-cell culture plate (positive control for ELISA). ELISA plates were incubated for 1 hour at room temperature, then washed again. All wells received 50 microliters per well of goat-anti-llama IgG-HRP conjugate (Bethyl) diluted 1:4000 in PBS containing 1% BSA and 0.05% Tween 20ELISA plates were incubated again for 1H at room temperature, washed and developed using 50 μl enhanced K-Blue TMB Substrate (Neogen) per well. The reaction was stopped by adding 50 μl of 2M H₂SO₄ per well and optical density at 450 nm of all wells was measured using an ELISA reader. As indicated above, goat-anti-llama IgG does not have selectivity towards either 4-chain or heavy chain immunoglobulin and this ELISA had been determined previously to detect llama IgG1, -2 and -3 with similar sensitivity.

Wells developing colour intensity (expressed as OD) twice over background were considered positive for immunoglobulin production. As background reference, the OD of ELISA wells corresponding to the two wells per plate having received only irradiated EL4-B5 cells and depleted TSN but no B-cells and not having received additional serum in the ELISA plate was used.

Thus, the identity of wells containing successfully stimulated B-cells was determined. The percentage of wells positive for B-cell outgrowth could then be calculated by dividing the number of positive wells by the number of screened wells having received sorted B-cells (92 per plate). Results are indicated in Table 14, below.

In order to determine which of these outgrowing B-cell cultures derived from B-cells reactive to TNF, the conditioned medium supernatant of these wells were screened for reactivity to TNF. To do so, Nunc Maxisorp plates were coated overnight at 4° C. using 2 microgram/ml of neutravidin in PBS. Plates were washed and then blocked using 1% casein in PBS for 1 hr at room temperature. Subsequently, plates were washed and coated overnight at 4° C. with 50 microliter/well of biotinylated recombinant human TNF diluted 1:2000 in PBS/1% casein. Plates were washed again and received 30 μl of B-cell culture supernatant diluted 1:2 with 30 μl 1% casein/PBS and incubated 1 hour at room temperature. Goat-anti-llama IgG-HRP conjugate (Bethyl) was used as secondary antibody and ELISA plates were then developed, read and evaluated similar to the procedure described for the previous ELISA.

Thus, the identity of wells containing outgrowing B-cells reactive to TNF was determined. The percentage of outgrowing B-cell cultures was calculated by dividing the number of anti-TNF reactive positive scoring wells by the number of screened outgrowing B-cell cultures. Results are indicated in Table 14, below, as percentage TNF reactive wells within the population of outgrowing B cell cultures.

TABLE 14
ELISA screening of growth and TNF reactivity B-cell cultures

| | % growth | % TNF reactive |
|---|---|---|
| TNF selections | | |
| TNF MACS (PE−/PI−) | 57 | 13 |
| TNF Plate (PE−/PI−) | 60 | 6 |
| Ig selection | | |
| Ig selected (PE−/PI−) | 79 | 1 |

As can be observed, outgrowth at the current cell seeding density of 3 cells/well was well over 50% in all plates. Limiting dilution experiment statistics demonstrate that even at this cloning efficiency, the large majority of outgrowing wells do not result from “multiple-hit”, that is, result from a single outgrowing B-cell per well and not more than one (out of three deposited in the well). Given the high cloning efficiency, reduction of seeding density to even fewer cells per well without losing practically useful outgrowth levels is feasible and will further reduce (at 2 cells/well) or eliminate (at 1 cell/well) these potential multiple hit wells. Also obvious is that selection on antigen resulted in highly significant enrichment for antigen specific B-cells, as non-specific B-cell isolation did not result in more than 1% of antigen reactive B-cell cultures versus 13 and 6% for MACS and plate-mediated TNF selection on the same cell sample, respectively.

In order to determine which of these outgrowing, TNF reactive B-cell cultures derived from B-cells producing heavy chain type antibodies and which ones conventional antibodies, the conditioned medium supernatant of TNF reactive wells were re-screened for reactivity to TNF but using IgG1, -2 and -3 selective monoclonal antibodies (obtained from Cornell University, NY, described in Daley et al. Clin Diagnos Lab Immunol 2005 12:380) as secondary detection reagents instead of the non-selective goat-anti-llama IgG polyclonal antiserum from Bethyl. To do so, three replica Nunc Maxisorp plates were coated with neutravidin, blocked, re-coated with biotinylated TNF and incubated with diluted conditioned supernatant of outgrowing B-cell culture supernatant as described for the previous ELISA. However, instead of using goat-anti-llama IgG-HRP conjugate (Bethyl), 1 microgram/ml of either purified mouse-anti-llama IgG1, anti-IgG2 or anti-IgG3 (clones 27E10, 19D8 and 8E1, respectively) was used as secondary antibody in each of the three replica ELISA plates, respectively. As these were not directly conjugated to HRP, plates were washed after a 1 hour incubation step at room temperature and incubated with 50 microliter per well of rabbit-anti-mouse-HRP conjugate (DakoCytomation) diluted 1:5000. This tertiary antibody conjugate was determined previously not to bind directly to any type of llama IgG, thus assuring the assay's selectivity was not biased by the use of this reagent. After this additional 1 hour incubation at room temperature, the ELISA plates were again washed, developed, read and evaluated similar to the procedure described for the previous ELISAs.

Thus, the type of TNF reactive immunoglobulin produced in wells already known to contain outgrowing B-cells reactive to TNF was determined. Results are indicated in Table 15, below.

TABLE 15
ELISA screening immunoglobulin type produced by TNF
reactive B-cell cultures

| | |
|---|---|
| 143 | TNF reactive cultures |

| | | |
|---|---|---|
| 138 | SN screened | 97% |
| 62 | IgG1+ | 45% |
| 3 | IgG2+ | 2% |
| 2 | IgG3+ | 1% |
| 2 | IgG1/2 | 1% |
| | double+ | |
| 73 | Undetermined | 53% |

As can be observed in Table 15, roughly half of all TNF reactive B-cell containing wells could be allocated to a given type of immunoglobulin using a convenient, straightfoward ELISA assay on the conditioned supernatant. Depletion of 4-chain producing B-cells during the cell sort prior to B-cell stimulation proved to be incomplete using the negative gating strategy based on immunoglobulin light chain staining employed here. It is clear to those skilled in cell sorting that the use of a positive rather than negative gating strategy and using species specific rather than crossreactive reagent to do so generally yields better selectivity. Clearly, the llama IgG2 and -3 specific mouse monoclonals used here for ELISA screening of the B-cell culture supernatants constitute a highly suitable set of reagents to do so. Lastly, it also worth noting the mixed signal cultures were both derived from 6 cell/well culture plates, where due to the very high outgrowth (˜80% of all B-cell seeded wells) the risk for obtaining some multiple hit wells was highest. No such mixed cultures were evident at 3 cells/well, and as indicated before, further reduction of cell seeding density to 2 or 1 cell/well will reduce or eliminate the chances of multiple hit cultures, respectively.

### Example 20

To confirm the ELISA based typing of B-cell culture produced immunoglobulins matches the genetic information which can be recovered from these cultures, we screened the conditioned supernatant of the B-cell cultures described in Example 19 using an ELISA which detects production of heavy chain or 4-chain type immunoglobulin, regardless of its reactivity towards TNF. To do so, three replica ELISA plates were coated with goat-anti-llama IgG (Bethyl), blocked, incubated with diluted B-cell culture conditioned medium as in the first ELISA procedure described in Example 19 but detected in replica plates 1, 2 and 3 using the llama IgG1, -2 and -3 selective mouse monoclonals, respectively, and developed and analyzed as for the immunoglobulin typing TNF capture ELISA described in the same example. Thus, we were able to unambiguously identify the type of immunoglobulin produced in 83% of all cultures producing relatively high amounts of immunoglobulin (OD>0.3 in any of the three parallel ELISAs).

To verify whether this ELISA based typing is consistent with the immunoglobulin mRNA sequences which can be rescued from these B-cell cultures, total RNA was isolated from selected TRIzol lysed B-cell culture cell pellets according to the manufacturer's instructions (Invitrogen) and a single-step RT-PCR reaction was performed (Superscript III One-step RT-PCR) using only a small portion of the RNA isolated. The reaction was set up using a primer set (forward: immunoglobulin FR1 specific, reverse: CH2 domain specific) which can amplify both 4-chain immunoglobulins and heavy chain immunoglobulins from llama, but yields PCR products of distinct length when amplifying either type. Thus, by detecting the presence of PCR products on an SYBR Green stained agarose gel (Invitrogen) after the RT-PCR reaction, the feasibility of rescuing the variable region gene sequence from B-cell cultures in a single step reaction is proven, whereas determination of the length of the product(s) on the same gel provides information on the type of immunoglobulin (4-chain versus heavy chain) encoded by the rescued gene fragment.

Amplicons could be obtained from all screened B-cell cultures, using a single step RT-PCR reaction on a fraction of the available RNA. Concordance of RT-PCR typing results with unambiguous ELISA typing where available are summarized in Table 16, below. “C” in the RT-PCR column denotes detection of an amplicon derived from conventional 4-chain type immunoglobulins (corresponding to IgG1), “V_{HH}” denotes an amplicon derived from either IgG2 or IgG3 heavy chain immunoglobulins.

TABLE 16
ELISA and RT-PCR immunoglobulin typing concordance

| | by ELISA | by RT-PCR |
|---|---|---|
| | IgG1 | C |
| | 4 | 4 |
| | IgG1 + 2 | C + V_{HH} |
| | 2 | 2 |
| | IgG2 | V_{HH} |
| | 7 | 6 (1 C) |
| TOTAL: | 13 | 12 |

Thus, variable region sequences can reliably be rescued from stimulated B-cell cultures using a single-step RT-PCR reaction and in 12 out of 13 B-cell cultures, ELISA typing provided correct information on the type of immunoglobulin sequence the expanded B-cells produce. Thus, needless amplification of cultures containing conventional B-cells or mixtures of conventional and non-conventional B-cells can be avoided upfront. As noted in the example before, the mixed signal cultures were both derived from 6 cell/well cultures; no such double signals were obtained from the many more 3 cell per well culture plates screened. Further reduction of cell seeding density will further reduce or completely remove the risk of such multiple hits.

### Example 21

### Separating Activated Cells From Non-Activated Cells

Individual B-lymphocytes carry randomly rearranged immunoglobulin genes, which impart different antigen specificities to the corresponding protein produced by the various B-cell clones. As rearrangements arise in all clones, and large numbers of B-cells are continuously produced throughout life, the relative abundance of any given rearrangement versus the total number of B-cells is very low.

By immunization or through natural immunity to previous infections, animals convert from an immunologically naive animal state to an immunized state. During this process, B-cell clones reacting to the antigen in question are “activated” and undergo a massive expansion in numbers whereas non-reactive cells do not, leading to a higher relative abundance of the antigen-reactive B-cells in the whole population. Concurrently, affinity maturation occurs, whereby antigen-reactive B-cells undergo cycles of mutations in the immunoglobulin gene and selection of the higher affinity variants resulting from these mutations. Thus, immunization leads to both an increased relative abundance of antigen-reactive B-cells, as well as to higher affinity antibody producing B-cells.

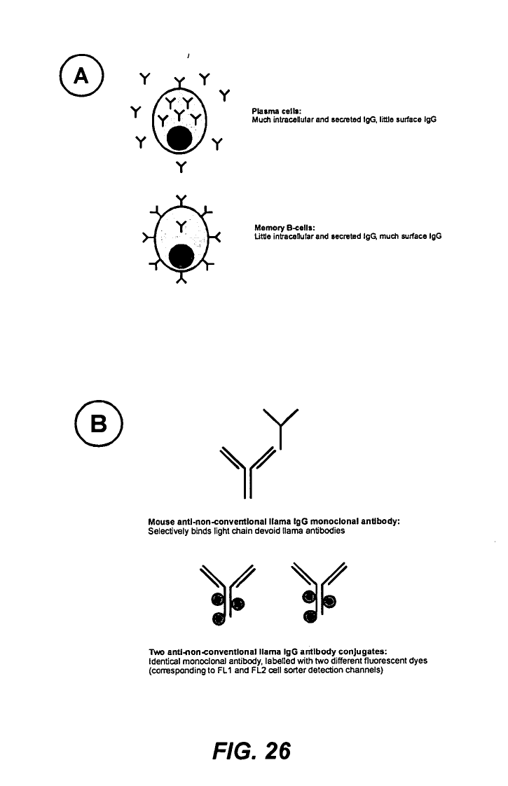

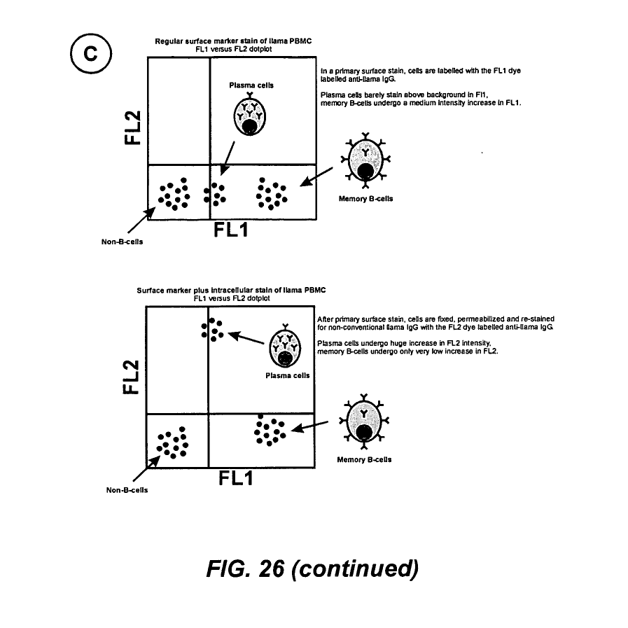

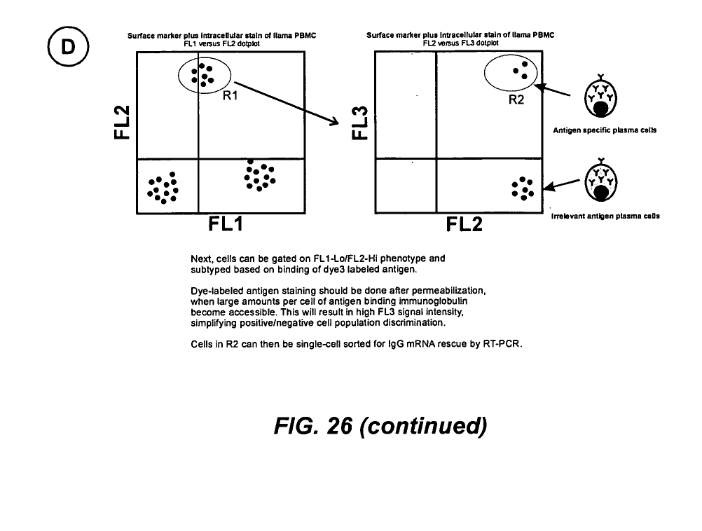

Activated B-cells (“plasma cells”) produce and secrete large amounts of soluble immunoglobulin, but display only minor quantities of this immunoglobulin on their cell membrane. A fraction of previously activated B-cells will become memory B-cells, which do not produce and secrete appreciable amounts of immunoglobulin, but display an appreciable number of antibodies on their cell membrane. See FIG. 26A.

Cell surface display of immunoglobulin allows the experimenter to enrich these from cell samples containing only a low abundance of relevant cells, based on antigen binding to the surface immunoglobulin. We have previously described how to use this method to isolate B-cells encoding non-conventional (light chain devoid) immunoglobulins binding an antigen of interest. However, as these memory B-cells do not actively produce immunoglobulin, they contain only minor amounts of the immunoglobulin encoding mRNA. As a result, extremely sensitive but erratic methods such as nested RT-PCR are required to isolate the gene directly from single sorted cells. Alternatively, the individual sorted memory B-cells can be re-activated in vitro using various known stimulation methods, such as CD40L transfected fibroblasts, feeder/stimulator cells such as the EL4-B5 cell line, or recombinant CD40L. Such re-activated cells divide (yielding ˜100 clonally expanded cells) as well as produce strongly increased amounts of immunoglobulin mRNA per individual cell. This dual target mRNA amplification step enables convenient and reliable single-step RT-PCR recovery of the immunoglobulin gene sequence, but does require additional time to allow for in vitro activation of B-cells. Also, in vitro stimulation of large panels of individual B-cells requires considerable skill.

We will here describe a method for the direct isolation by cell sorting of individual plasma cells producing non-conventional immunoglobulins binding an antigen of interest. As this method yields plasma cells rather than quiescent memory B-cells, these are much more amenable to single-step RT-PCR gene recovery. Thus, in vitro B-cell activation after sorting the cells of interest is no longer required. As a result, less time, effort and expertise is required to obtain large numbers of lead molecules.

The method relies on the use of a single reagent, preferably but not necessarily a monoclonal antibody, which specifically binds camelid non-conventional immunoglobulins in the presence of conventional immunogobulins (FIG. 26B, upper panel). Such reagents are readily available for lama glama, as described in Daley et al. (Clin Diagn Lab Immunol. 2005 March;12(3):380-6.). These hybridomas provide an unlimited supply of monospecific mouse-anti-llama non-conventional IgG immunoglobulin, which can readily be conjugated to a vast panel of currently available fluorochromes using standard antibody conjugation techniques. Thus, it is now trivial to generate two conjugates from the same monoclonal antibody, but carrying different fluorochromes (FIG. 26B, lower panel).

When staining unfractionated llama peripheral blood mononuclear cells using the first conjugate, only surface immunoglobulin will be detectable. Thus, if this sample should now be analyzed, memory B-cells will readily be discriminated from non-B-cells, whereas plasma cells will be barely more fluorescent than non-B-cells (FIG. 26C, upper panel).

However, the thus stained cell sample can also be fixed and permeabilized, using methods well known to those experienced in flow cytometry cell analysis and sorting. Fixation will retain the surface bound stain on the cell surface, while also permitting subsequent permeabilization of the cell using detergents such as saponin without causing complete cell lysis. Fixation also causes intracellular molecules to be retained within the cell, despite the cell membrane being opened up. Permeabilization allows large molecules such as monoclonal antibody/fluorochrome conjugates access to the intracellular environment, which is impossible prior to permeabilization. Thus, the large amounts of intracellular immunoglobulin present in plasma cells can be stained using the second dye conjugate of the same anti-non-conventional llama IgG monoclonals as was used to stain surface immunoglobulin. The high local abundance of the target molecule ensures an intense fluorescent signal can be obtained, despite access to it being limited as compared to surface immunoglobulin (FIG. 26C, lower panel).

Descriptions for reagents required for gentle fixation and permeabilization can readily be found in the current literature (usually paraformaldehyde and saponin supplemented buffered saline, respectively), but many highly optimized proprietary ready-made buffers are also available commercially. A limited proof-of-concept can also be found in the literature, as detection of intracellular light chain by flow cytometry has been documented many years ago as a tool for diagnosis of B-cell lymphomas; see Bardales et al. (J Histochem Cytochem. 1989 January;37(1):83-9.) as an example.

Thus far, we have defined a general method for identifying non-conventional type antibody producing plasma cells in mixed llama cell populations, requiring only a single specific monoclonal antibody. However, it is likely the fraction of plasma cells producing antibodies binding the antigen of interest constitute only a minority of this population. Although this fraction can be increased significantly by boost immunization of the animal shortly prior to blood sampling, it would be desirable to identify these cells within the total population of plasma cells nevertheless.

Many antigens of therapeutic interest are of a similar size as immunoglobulins or smaller. For those larger than that, only antibodies binding a particular small subdomain are relevant and the subdomain can be obtained in isolation or as a fusion protein (for instance, fused to immunoglobulin constant regions). It should therefore not be specifically problematic to stain permeabilized cells with antigen pre-labeled with a third fluorescent dye, as these smaller molecules may gain access to the intracellular compartment even more readily then anti-immunoglobulin monoclonal antibody fluorescent conjugates. Alternatively, one may use biotinylated antigen to stain the permeabilized cells and detect using fluorophore conjugated streptavidin in a second step reaction. Thus, purified antigen directly or indirectly conjugated to a third fluorescent dye may be used to specifically label the subpopulation of plasma cells producing immunoglobulin binding the antigen (FIG. 26D). By electronic gating on the total plasma cell population prior to subgating the antigen-dye staining population, one may avoid erroneously including quiescent antigen specific B-cells which might bind the antigen-dye conjugate on their surface immunoglobulin (FIG. 26D). As a local concentration of immunoglobulin intracellularly is very high in plasma cells, relatively large amounts of antigen binding sites are available and high staining intensities may be obtained, further simplifying unambiguous identification of the positive population versus background staining.

Once a specific method is established for identifying a cell population of interest using a flow cytometer, there remains no technical barrier for isolation of the population in question using a flow cytometric cell sorter. Individual plasma cells producing non-conventional antigen-reactive antibody can be sorted directly into microtiter plate wells for mRNA isolation, reverse transcription to cDNA and PCR amplification or direct, single step RT-PCR amplification of the clone's specific immunoglobulin gene.

Rescue of the immunoglobulin gene from sorted cell mRNA can be expected to pose the largest technical hurdle to this procedure. Nonetheless, RNAse inhibiting reagents are now available and mRNA recovery by RT-PCR after cell sorting using these has been documented in Barrett et al. (Nature Genetics 1999 23: 32 and Biotechniques. 2002 32(4):888-90, 892, 894, 896). As plasma cells are particularly rich in immunoglobulin mRNA, this step should not hinder method.

The possibilities for refinement on this general procedure outline are plentiful. For instance, identification of a rare cell population generally benefits from identifying the population of interest using two different dyes. In this particular case, antigen binding defines the lowest abundance cell population. By staining the cells with a mix of antigen conjugated to two different fluorochromes, false positive sorts can be reduced.

The relative abundance of plasma cells in general, and antigen specific ones in particular, can be increased by boosting a pre-immune animal with an extra immunization shortly before the blood sample to be sorted is drawn. Analytical, rather than preparative, flow cytometry using the method described here can be used to guide optimization of the immunization schedule.

It is expected that permeabilized plasma cells contain much greater total numbers of immunoglobulin epitopes available for anti-llama IgG binding than permeabilized B-cells do (the total being surface displayed plus intracellularly contained). Therefore, it may be feasible (depending on staining intensity and positively staining populations peak widths) to clearly discriminate these populations as two defined peaks in a single fluorescence readout channel, using only a single monoclonal antibody conjugated to a single dye to stain fixed/permeabilized cells in a one-step reaction. This would have the benefit of greater procedural simplicity, but also of requiring one less fluorescence readout channel from the cell sorter device—reducing hardware requirements and easing the problem of multicolor compensation.

### Example 22

### TNF Binding B-Cells can be Detected by FACS and Enriched for Using Magnetic Beads

Immunization, Cell Isolation and Antigen Enrichment

A healthy adult, male llama (#055) was immunized and boosted repeatedly with recombinant human TNFalpha (produced by Ablynx Nev.) emulsified in Stimune adjuvant (Cedi Diagnostics, Lelystad, Netherlands). Peripheral blood leucocytes (PBL) were obtained from a freshly drawn blood sample and depleted of monocytes as described in Example 19, above. In parallel, PBL were isolated from a fresh blood sample of an unimmunized animal (#068). The monocyte depleted cells from the immunized animal were labeled with biotinylated recombinant human TNFalpha (produced by Ablynx Nev.) and selected using anti-biotin MACS beads as described in the same example. An aliquot of monocyte depleted PBL of the immune animal prior to selection was retained for control purposes.

FACS Analysis of Cell Preparations

Next, the non-immune animal PBL, immune animal PBL not selected using MACS and the immune animal PBL fraction selected using biotinTNF/anti-biotin MACS beads were stained for FACS analysis and sorting according to standard methods known to those skilled in the art. The probe/dye mixture employed was Alexa488 conjugated recombinant human TNFalpha (produced by Ablynx Nev., Alexa488 SMCC obtained from Invitrogen), phycoerythrin (PE) labeled monoclonal mouse-anti-llama IgG2 clone 19D8, PE labeled monoclonal mouse-anti-llama IgG3 clone 8E1, allophycocyanin (APC) labeled mouse-anti-llama IgG1 clone 27E10 (hybridomas described in Daley et al. were grown and monoclonal antibodies were purified and conjugated by Ablynx Nev. to SMCC-preactivated PE or APC (Prozyme, San Leandro, Calif.)) and live/dead discriminator dye TOPRO3 (Invitrogen).

Analysis and sorting was performed on a 488 nm/635 nm dual laser BD FACSaria cell sorting system equipped with a cooled automated cell deposition unit (ACDU); proper forward, side scatter and fluorescence photomultiplier settings as well as compensation were set using unstained or single-stained immune animal PBL samples according to standard methods well known to those skilled in the art.

Analysis and sorting was set up as a series of nested, “drill-down” gates to first identify the lymphocyte subset within the cell samples using forward/side scatter dotplots, eliminate doublet measurements within this gate using forward scatter pulse area versus pulse height, then further eliminate doublets using side scatter pulse area versus pulse height, eliminate both dead cells and conventional four-chain antibody cell surface positive cells based on APC/TOPRO3 channel fluorescence signal intensity, identify heavy chain immunoglobulin (IgG2 or -3) surface positive B-cells within the remaining population based on PE fluorescence signal intensity and, finally, identify antigen binding B-cells within the heavy chain IgG surface positive population based on Alexa488 fluorescence intensity.

An arbitrary region was drawn on the high fluorescence intensity side of the Alexa488 fluorescence histogram so that few events would fall within the region in the non-immune animal control sample. The percentage of cells falling within this region versus the total number of cells analysed within this pre-gated heavy chain antibody positive B-cell population is shown in Table 17.

TABLE 17

| Blood sample origin | MACS enrichment | TNF-A488+ |
|---|---|---|
| Non-immune animal 068 PBL | unenriched | 2.3% |
| Immune animal 055 PBL | unenriched | 1.9% |
| Immune animal 055 PBL | pre-enriched | 3.1% |

As can clearly be observed, a similar number of heavy chain positive B-cells cells bind fluorescent TNF in immune and non-immune animal PBL, but more cells from the same blood sample bind fluorescent TNF after enrichment using biotinylated antigen and anti-biotin magnetic microbeads.

### Example 23

### FACS Staining by Fluorescent Antigen is Antigen Specific

Immunization, Cell Isolation, Storage and Thawing

An adult male llama (#054) was immunized and boosted repeatedly with recombinant human TNFalpha (produced by Ablynx Nev.) emulsified in Stimune adjuvant (Cedi Diagnostics, Lelystad, Netherlands). Peripheral blood leucocytes (PBL) were obtained from a freshly drawn blood sample, depleted of monocytes as described above and stored as a series of aliquots in 90% FCS/10% DMSO freeze medium under liquid nitrogen. A vial of such frozen cells was thawed quickly in a waterbath at 37° C. and washed repeatedly with RPMI1640/10% FCS medium.

FACS Analysis of Cell Preparations

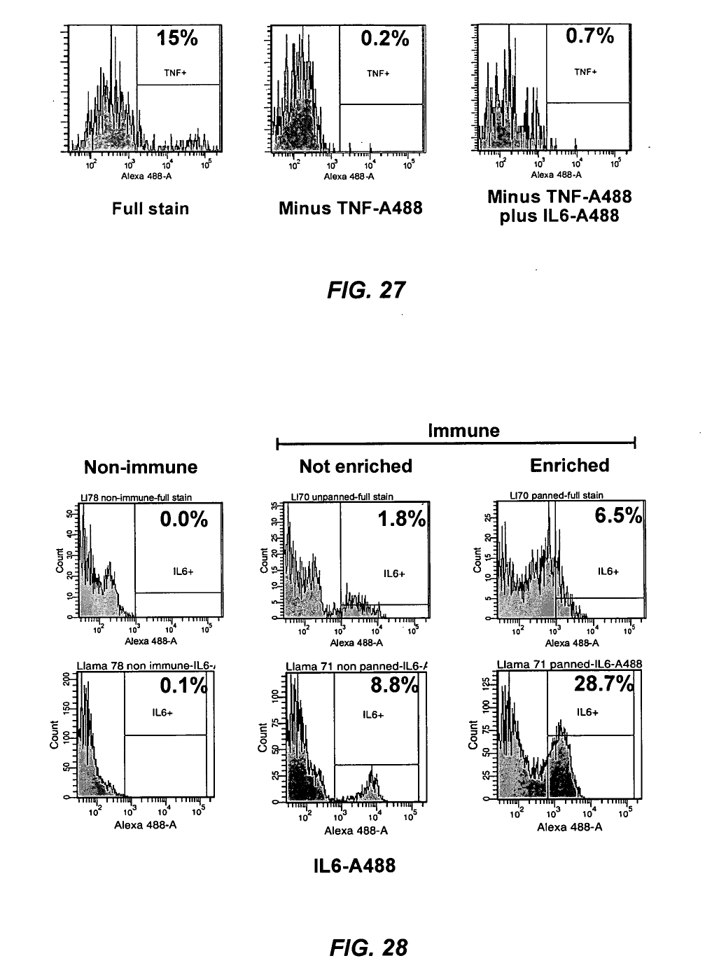

These immune animal PBL were stained for FACS analysis using anti-IgG1 (APC labeled), -2 (PE labeled), -3 (PE labeled) mAbs and live/dead discriminator dye TOPRO3 according to the methods described in Example 22, above. Aliquots of the cell sample were also stained using Alexa488 labeled recombinant TNF, as described before, whereas parallel aliquots received all stains except fluorescent TNF or all stains except Alexa 488 labeled IL6 was substituted for Alexa 488 labeled TNF. Results are shown in FIG. 27.

We concluded 15% of live B-cells bearing heavy chain IgG on their surface bind the fluorescently labeled antigen with which the animal was immunized. This results was not due to optical spillover from other fluorescent dyes or autofluorescence, as cells from the same sample not stained with Alexa 488 labeled reagents only very rarely (0.2%) are sufficiently fluorescent to fall within the high fluorescence measurement region (labeled TNF+ on FIG. 27). Also, binding of the fluorescent antigen is truly specific for the antigen as staining of the cell sample with Alexa 488 labeled recombinant human IL6, an antigen the animal was not exposed to, does not result in significantly more staining (0.7%) than if no antigen was used for staining at all (0.2%). Therefore, sorting of B-cells with surface heavy chain IgG (IgG2 or -3) also binding fluorescently labeled antigen can be assumed to yield an relatively pure population of cells containing rearranged DNA and mRNA coding for truly antigen reactive IgG2 or -3 immunoglobulins. In turn, sorting of such cells and recovery of the VHH gene segments on an one-by-one cell basis would yield a collection of VHH genes, most of which would encode an antigen binding protein.

### Example 24

### Antigen Binding Heavy Chain B-Cells can be Sorted and Antigen Binding VHH Gene Segments can be Rescued from Individual Sorted B-Cells

FACS Sorting of Cell Preparation

Cells falling within the high fluorescence region of the Alexa488 histogram shown in Example 23 were sorted as individual cells into multiple 8-well PCR strips all containing 25 microliter of the 2× buffer provided with the Superscript III One-step RT-PCR kit (Invitrogen). Use of the particular doublet elimination gating strategy described above, in combination with the manufacturer's predefined “single-cell sorting mode”, effectively prevents multiple cells from being sorted into the same well. PCR strips were cooled to 4° C. using the ACDU cooling module throughout the sort period. One well per strip containing buffer was set up not to receive a cell. All PCR tubes were capped and snap-frozen at −80° C. immediately after finishing the sort.

RT-PCR Rescue of VHH Gene Segment

A master mix of NP-40 (Roche Diagnostics), primers and enzyme/nucleotide mix for both RT and PCR reactions was freshly prepared immediately before adding at 25 microliter per well to all wells of freshly thawed −80° C. stored PCR strips containing sorted B-cells, including the control wells not having received a cell. Both reverse transcription reaction and PCR amplification of the cDNA were performed sequentially in the same tube, using the same 3′ primer for both reactions. Primer sequences are shown in Table 18, the thermal cycler program in Table 19. All reactions were run on a T3 thermocycler (Biometra).

TABLE 18

SEQ ID

| | | |
|---|---|---|
| Primer | NO | Sequence (5′ to 3′ direction) |

| | | |
|---|---|---|
| 5′ | 51 | TGACTCGCGGCCCAGCCGGCCATGGCCGAGGTGCAGC |
| | | TGGTGGAGTCTGG |
| 3′ | 52 | GGTACGTGCTGTTGAACTGTTCC |

TABLE 19

| Step | Temperature (° C.) | Duration | | Cycling |
|---|---|---|---|---|
| 1 | 55 | 30 | minutes | 1x |
| 2 | 94 | 2 | minutes | 1x |
| 3 | 94 | 15 | seconds | 40x |
| 4 | 55 | 30 | seconds | |
| 5 | 68 | 45 | seconds | |
| 6 | 68 | 5 | minutes | 1x |

Once both RT and PCR reactions were run, an aliquot of the reaction mixture was analyzed on SYBR Safe stained agarose gel. 6 out of 20 of the one cell/well RT-PCR reactions were found to contain visually detectable product. All products were of the length expected for products of the nested primer set, and PCRs run on the wells not having received any cell were negative for product.

Cloning of Rescued VHH Gene Segments

Next, aliquots of the 6 PCR reactions found to contain an amplicon were purified using Qiagen PCR purification spin columns (Qiagen), digested using the Sfi I (New England Biolabs) and Eco 91I (Fermentas) restriction enzymes and digests were purified via agarose gel using Qiaquick gel extraction kit (Qiagen). 4 out of the 6 RT-PCR products gave visible bands on agarose gel after this purification step and were ligated into the purified, Sfi I/Eco 91 I double pre-digested pAX51 E. coli expression vector (custom vector, produced by Ablynx Nev., containing IPTG induced promoter, periplasmic space export leader signal N-terminal of the cloning site and a c-myc/6His dual fusion tag in-frame and C-terminal of the cloning site) using standard methods. Ligation mixtures were introduced into competent E. coli through electroporation and cells were plated out on petri dishes containing antibiotic selective medium. Individual colonies were then picked and bacteria were grown in small volumes of liquid selective medium overnight. All colonies were screened for the presence of plasmid insert using colony PCR with two primers annealing to sequences located shortly 5′ and 3′ of the cloning site, respectively, and detection of PCR amplicon length using SYBR Safe stained agarose gels. Gel analysis demonstrated all 4 double-digested, purified RT-PCR products were ligated successfully into the plasmid in this experiment.

Overnight cultures were prepared of colonies found to contain an insert and plasmids were purified from these using Qiagen plasmid miniprep spin columns (Qiagen). The PCR fragment inserts of the plasmids were sequenced from the minipreps using standard automated sequencing methods.

Sequencing data confirmed the ligated RT-PCR amplicons were V_{HH} gene sequences in all 4 clones. Also, colonies derived from different individual sorted B-cells were shown to contain non-identical sequences.

Screening of Antigen Reactivity of Cloned VHH Genes

Replicates of the same cultures used for sequencing were grown overnight and protein was extracted from the periplasmic space of the cells using standard methods. Fixed dilutions of crude E. coli extract were screened for binding in parallel on biotinylated TNF immobilized on neutravidin coated ELISA plates, biotinylated IL-6 immobilized on neutravidin coated ELISA plates or neutravidin coated ELISA plates, the latter two as negative controls. Binding was detected using an anti-c-myc tag mouse monoclonal antibody (Serotec), followed by an goat-anti-mouse IgG-HRP conjugate as second step reagent (Dako). Plate-bound HRP was detected using TMB (Pierce) as a chromogenic substrate. Background was defined as the optical density (OD) of ELISA plate wells not receiving E. coli extract but having received anti-c-myc and anti-mouse-HRP. ODs of wells having received E. coli extracts over six times the background OD value were considered positive.

4 recombinant VHHs out of 4 obtained from RT-PCR products from single sorted B-cells were found to bind immobilized biotin-TNF but not biotin-IL-6 or neutravidin plates without any immobilized biotinylated ligand. We therefore concluded all of these clones bind human TNF and do so specifically.

Screening of TNF Neutralization in a Sensitive TNF Bioassay

A new batch of protein extracts was prepared from all 4 clones by inducing protein overexpression using IPTG in new log-growth phase E. coli replicate cultures and purified from these crude protein extracts using nickel-chelating NTA resin (Qiagen) according to the manufacturer's instructions. Concentration of all purified protein preparations was determined using OD280 and the specific molar extinction coefficient of these clones calculated based on their protein sequence, in turn inferred from the previously determined nucleotide sequence. Serial dilutions with known concentrations of these recombinant VHHs were assayed for their TNF neutralization potential in an in vitro TNF bioassay. The TNFα sensitive mouse fibroblast cell line L929s was used for measuring the anti-TNFα activity of the selected nanobodies. At a sufficiently high concentration of TNFα in the medium, i.e. cytotoxic dose, L929s cells undergo necrosis. The inhibition of TNFα interaction with its receptor was determined by pre-incubating a series of antibody dilutions with a cytotoxic concentration of TNFα before adding the mixture to the cells. The presence of actinomycin D in the medium sensitises the cells further to TNFα, resulting in increased sensitivity of the bioassay for free TNFα. The L929 cells were grown to nearly confluency, plated out in 96-well microtiter plates at 5000 cells per well and incubated overnight. Actinomycin D was added to the cells at a final concentration of 1 μg/ml. Serial dilutions of the nanobodies to be tested were mixed with a cytotoxic concentration of TNFα (final assay concentration is 0.5 ng/ml or 15 IU/ml). After at least 30 minutes of incubation at 37° C., this mixture was added to the plated cells. Plates were incubated for 24 hours at 37° C. and 5% CO2. Cell viability was determined by use of the tetrazolium salt WST-1. Dose-response curves and EC50 values were calculated with Graphpad Prism. The assay has been configured to detect TNF neutralization by antibodies or fragments thereof with potencies 150 mM or better. Results are summarized in Table 20.

TABLE 20

| Clone reference | EC50 (nM) |
|---|---|
| 055 TNF NC1 | >150 |
| 055 TNF NC2 | >150 |
| 055 TNF NC3 | 19.6 |
| 055 TNF NC11 | >150 |

1 out of 4 of these clones was shown to protect L929s cells from a cytotoxic dose of TNF even in the presence of the TNF-induced cell death sensitizing agent actinomycin D (EC50 of ˜20 nM). It cannot be excluded based on the results of this assay that the other three are neutralizing with potencies of over 150 nM. Therefore, we conclude at least some clones isolated using this method not only bind the antigen specifically, but can also inhibit its biological function.

### Example 25

### Isolation of IL6 Binding Nanobodies by Nanoclone

Immunization, Cell Isolation and Antigen Enrichment

Peripheral blood leucocytes (PBL) were obtained from freshly drawn blood samples from two healthy adult, male llamas (070, 071) immunized and boosted with recombinant human IL-6. Animals 070 and 071 were not sampled on the same day. Also, animal 070 was sampled at two timepoints. Blood was drawn and PBL were isolated from unimmunized animals 072 or 078 every time an immune animal was sampled. The monocyte depleted cells from immune animal samples were labeled with biotinylated recombinant human IL-6 (produced in-house) and selected using anti-biotin MACS beads as described in the previous example. As before, aliquots of monocyte depleted PBMC of the immune animal prior to bead selection were retained for control purposes.

FACS Analysis of Cell Preparations

Next, the non-immune animal PBL, immune animal PBL not enriched using MACS and the immune animal PBL fraction enriched for IL-6 binding B-cells using biotin-IL6/anti-biotin MACS beads were subjected to the same staining and FACS analysis procedure as described in the previous example, except that Alexa488 conjugated IL-6 was substituted for Alexa488 conjugated TNF. As before, live, single heavy chain immunoglobulin surface IgG positive B-cells were isolated based on complex gating strategy, after which fluorescent antigen binding within this population was visualized in a histogram display of the antigen-associated fluorescence within this population. An arbitrary high fluorescence region was defined in this histogram, chosen to minimize positivity of the non-immune animal sample. Results are shown in FIG. 28; numbers in the histograms denote the percentage of IL6-Alexa488 positivity scoring cells versus the total population of the parent gate (live, heavy chain IgG surface positive B-cells).

Based on these experiments, we concluded a well defined IL6-binding heavy chain IgG positive B-cell population could be distinguished from the majority of IL6 non-binding heavy chain IgG producing B-cells in the immune animal. The IL6-binding IgG positive B-cells constituted ˜2 to 9% of the heavy chain repertoire. This subfraction of B-cells could be enriched for by at least a factor of 3 by panning the immune animal cells using magnetic beads. Little background binding was observed of fluorescent human IL6 to irrelevant heavy chain B-cells, as defined by the staining pattern of non-immune animal B-cells.

FACS Analysis of Cell Preparations is Highly Reproducible

The above staining procedure was repeated on a new blood sample of the same immune animal (070) drawn one week later. The animal did not received IL6 booster injections in the time between the two blood draws. In parallel to the PBL isolation from the immune animal, PBL were isolated from a fresh blood sample of previously unsampled non-immune animal 072. The FACS analysis results are summarized alongside the previous ones in Table 21.

TABLE 21

% IL6-A488+ heavy
chain IgG+ B-cells

| Animal | 1^{st} experiment | 2^{nd} experiment |
|---|---|---|
| Non-immune 078 | 0.1 | 0.0 |
| Non-immune 072 | 0.3 | — |
| Immune 070 | 1.7 | 1.8 |

Table 21 clearly shows the percentage of IL6-binding B-cells determined this way to be highly reproducible from day to day over this short period, as 1.7% of heavy chain B-cells was found to positive in llama 070 on one day versus 1.8% in a blood sample of the same animal obtained one week later. Background binding of the labeled antigen to the new non-immune control animal heavy chain IgG positive B-cells was also reproducibly low again (0.3% in animal 072 and 0.1 or 0.0% for animal 078 on two dates).

RT-PCR Rescue of VHH Gene Segment

Individual Alexa 488 labeled IL6 binding heavy chain IgG displaying B-cell were sorted on an individual cell/well basis into cooled PCR strips containing 25 microliter of 2× RT-PCR buffer, as described in Example 24. One well per strip was set up not to receive a cell and all strips were frozen at −80° C., all as described before.

A master mix of NP-40 (Roche Diagnostics), primers and enzyme/nucleotide mix for both RT and PCR reactions was freshly prepared immediately before adding at 25 microliter per well to all wells of freshly thawed −80° C. stored PCR strips containing sorted B-cells, including the control wells not having received a cell. Both reverse transcription reaction and PCR amplification of the cDNA are performed sequentially in the same tube, using the same 3′ primer for both reactions. Both primers as well as the thermal cycler program were identical to those described in Example 24. Once both RT and PCR reactions were run, an aliquot of the reaction mixture was transferred into new PCR tubes containing a PCR master mix with semi-nested primers. A blend of two DNA polymerases containing a proofreading enzyme (Expand High Fidelity PCR system, Roche Diagnostics) was used for the semi-nested PCR to avoid introducing PCR artefacts in the final product. Primer sequences are shown in Table 22, the thermal cycler program in Table 23. The reactions were run on the same make and model thermal cycler as the primary RT-PCR.

TABLE 22

SEQ ID

| | | |
|---|---|---|
| Primer | NO | Sequence (5′ to 3′ direction) |

| | | |
|---|---|---|
| 5′ | 53 | TGACTCGCGGCCCAGCCGGCCATGGCCGAGGTGCAGC |
| | | TGGTGGAGTCTGG |
| 3′ | 54 | TGAGGAGACGGTGACCTG |

TABLE 23

| Step | Temperature (° C.) | Duration | | Cycling |
|---|---|---|---|---|
| 1 | 94 | 5 | minutes | 1x |
| 2 | 94 | 15 | seconds | 25x |
| 3 | 55 | 30 | seconds | |
| 4 | 72 | 45 | seconds | |
| 5 | 72 | 10 | minutes | 1x |

Once the PCR reactions were run, an aliquot of the reaction mixture was analyzed on SYBR Safe stained agarose gel. 19 out of 21 one cell/well RT-PCR reactions were found to contain visually detectable product. All products were of the length expected for products of the nested primer set, and PCRs run on the wells not having received any cell were negative for product.

Cloning of Rescued VHH Gene Segments

Next, aliquots of 8 PCR reactions found to contain an amplicon were purified and cloned into an expression vector using the same methods as described in Example 24. Plasmid was purified from overnight cultures and sequenced as described in the same example.

As before, sequencing data confirmed the ligated RT-PCR amplicons were V_{HH} gene sequences in all clones and colonies derived from different individual sorted B-cells were shown to contain non-identical sequences.

Some of the V_{HH} sequences generated using the methods described herein are described in the co-pending US provisional application by applicant entitled “Nanobodies™ against IL-6 and polypeptides comprising the same”, which has the same filing date as the present application.

Screening of Antigen Reactivity of Cloned VHH Genes

Crude protein extracts were prepared from overnight grown replicates of the same colonies used for sequencing. Extracts were tested for binding on biotinylated IL-6 captured on neutravidin coated ELISA plates. As controls, replicates were tested for binding on biotinylated TNF captured on neutravidin coated plates, or neutravidin coated plates not having captured any biotinylated antigen. Background and significant binding to the plates were defined as discussed in Example 24.

Results indicated 4 out of 8 crude extracts tested bound to immobilized IL6 but not TNF or control neutravidin-only coated ELISA plates. We therefore concluded at least half of the clones obtained bind to the antigen used for selecting the B-cells serving as template in the RT-PCR reaction, and do so specifically as they do not bind several irrelevant proteins.

Aliquots of the same crude E. coli extracts were also boiled in reducing Laemmli sample buffer and loaded onto an SDS-PAGE gel. The gel was run, electroblotted onto nitrocellulose membrane and the membrane was probed using an anti-c-myc fusion tag mouse monoclonal antibody followed by an alkaline phosphatase (AP) conjugated anti-mouse polyclonal antibody according to standard methods known to those skilled in the art. Analysis of the blot after AP substrate development indicated 3 of the 4 extracts found not to bind immobilized IL6 in ELISA contained barely detectable to no recombinant VHH. This could be explained as these were derived from cultures not having received the protein production inducer IPTG. Clearly, extracts found not to contain IL6 binding activity in the IL6 binding ELISA but not containing detectable levels of recombinant protein may represent clones binding IL6 if sufficient recombinant protein were produced and used in the binding assay. Thus, although 4 out of 8 extracts were found not to bind IL6 in the binding ELISA, the fraction of clones obtained from individual IL6 binding, sorted and cloned B-cells may be higher than 50%.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

All of the references described herein are incorporated by reference, in particular for the teaching that is referenced hereinabove.

We claim:

1. Method for generating or cloning a nucleic acid or nucleotide sequence that encodes a heavy chain antibody or an antigen-binding fragment thereof, wherein said heavy chain antibody or antigen-binding fragment is directed against a specific antigen, said method comprising the steps of:
a) providing a sample or population of cells from a Camelid immunized with said antigen, wherein said sample or population of cells comprises at least one cell that expresses or is capable of expressing a heavy chain antibody directed against said antigen;
b) isolating from said sample or population said at least one cell that expresses or is capable of expressing a heavy chain antibody directed against said antigen; and
c) obtaining from said at least one cell a nucleic acid or nucleotide sequence that encodes a heavy chain antibody directed against said antigen or that encodes an antigen-binding fragment thereof directed against said antigen.

2. Method according to claim 1, in which step b) comprises any suitable combination of the following steps:
b-1) separating cells that express antibodies from cells that do not express antibodies;
b-2) separating cells that express antibodies against the desired antigen from cells that express antibodies directed against other antigens; and
b-3) separating cells that express heavy chain antibodies from cells that express conventional 4-chain antibodies;
in which said steps may be performed in any order and in which each two or all three of said steps may also be performed as a single step.

3. Method according to claim 1, in which, after step b) and prior to step c), the individual cells are cultivated and/or stimulated to express or produce antibodies, after which the medium or supernatant from each individual cell is screened for the presence of a heavy chain antibody against the desired antigen.

4. Method for generating or cloning a nucleic acid or nucleotide sequence that encodes a heavy chain antibody or an antigen-binding fragment thereof, wherein said heavy chain antibody or antigen-binding fragment is directed against a specific antigen, said method comprising the steps of:
a) providing a sample or population of cells from a Camelid immunized with said antigen, wherein said sample or population of cells comprises at least one cell that expresses or is capable of expressing a heavy chain antibody directed against said antigen;
b) isolating from said sample or population, as individual cells or as a set of individual cells, cells that express heavy chain antibodies;
c) screening said individual cells or set of individual cells for the expression of a heavy chain antibody directed against said antigen; and
d) obtaining from said at least one cell a nucleic acid or nucleotide sequence that encodes a heavy chain antibody directed against said antigen or that encodes an antigen-binding fragment thereof directed against said antigen.

5. Method according to claim 4, in which, in step c), the individual cells are cultivated and/or stimulated to express or produce antibodies, after which the medium or supernatant from each individual cell is screened for the presence of a heavy chain antibody against the desired antigen.

6. Method for generating or cloning a nucleic acid or nucleotide sequence that encodes a heavy chain antibody or an antigen-binding fragment thereof, wherein said heavy chain antibody or antigen-binding fragment is directed against a specific antigen, said method comprising the steps of:
a) providing a sample or population of cells from a Camelid immunized with said antigen, wherein said sample or population of cells comprises at least one cell that expresses or is capable of expressing a heavy chain antibody directed against said antigen;
b) separating said sample or population of cells into a set of individual cells;
c) screening said set of individual cells for cells that express a heavy chain antibody directed against said antigen; and
d) obtaining, from said at least one cell that expresses a heavy chain antibody directed against said antigen, a nucleic acid or nucleotide sequence that encodes a heavy chain antibody directed against antigen or that encodes an antigen-binding fragment thereof directed against said antigen.

7. Method according to claim 6, in which, in step c), the individual cells are cultivated and/or stimulated to express or produce antibodies, after which the medium or supernatant from each individual cell is screened for the presence of a heavy chain antibody against the desired antigen.

8. Nucleic acid, identified, selected, generated and/or cloned using the method of claim 1.

9. Nucleic acid according to claim 8, which is in the form of a genetic construct.

10. Genetic construct, comprising a nucleic acid that has been identified, selected, generated and/or cloned using the method of claim 1, and optionally one or more further elements of genetic constructs known per se.

11. Method for producing a heavy chain antibody or antigen-binding fragment thereof, said method comprising expressing a nucleic acid according to claim 8, in a suitable host cell or host organism.

12. Host cell or host organism, comprising a nucleic acid according to claim 8.

13. Heavy chain antibody or antigen-binding fragment thereof, encoded by a nucleic acid according to claim 8.

14. Heavy chain antibody or antigen-binding fragment thereof according to claim 13, wherein said antigen-binding fragment is a V_{HH} domain.

15. Nanobody, the amino acid sequence of which is based on the amino acid sequence of the V_{HH} domain according to claim 14.

16. Protein or polypeptide, containing or comprising at least one V_{HH} domain according to claim 14.

17. Nucleic acid or nucleotide sequence, encoding a protein or polypeptide according to claim 16.

18. Nucleic acid according to claim 17, in the form of a genetic construct.

19. Genetic construct, comprising a nucleic acid according to claim 17, and optionally one or more further elements of genetic constructs known per se.

20. Method for producing a protein or polypeptide containing or comprising at least one V_{HH} domain and/or at least one Nanobody, said method comprising expressing a nucleic acid according to claim 17, in a suitable host cell or host organism.

21. Host cell or host organism, comprising a nucleic acid according to claim 17.
